(19) United States
(12) Patent Application Publication
(10) Pub. No.: US 2023/0181564 A1
(43) Pub. Date: Jun. 15, 2023

(54) Potent Tetrahydroquinolone Eliminates Apicomplexan Parasites

(71) Applicant: The University of Chicago, Chicago, IL (US)

(72) Inventors: Rima McLeod, Chicago, IL (US); Ernest Mui, Chicago, IL (US); Ying Zhou, Chicago, IL (US); Kurt Ristroph, Princeton, NJ (US); Robert Prud?homme, Princeton, NJ (US); Hernan Lorenzi, La Jolla, CA (US); Colin Fishwick, Leeds, Yorkshire (GB); Martin McPhillie, Leeds, Yorkshire (GB)

(21) Appl. No.: 17/926,030

(22) PCT Filed: May 18, 2021
(86) PCT No.: PCT/US2021/033019

§ 371 Date: Nov. 17, 2022

Related U.S. Application Data
(60) Provisional application No. 63/026,415, filed on May 18, 2020.

(51) Int. Cl. A61K 31/4709; A61K 9/10; A61P 33/06; A61K 31/519; A61K 9/51

(52) U.S. Cl. CPC A61K 31/4709; A61K 9/10; A61P 33/06; A61K 31/519; A61K 9/5161; A61K 9/5146; B82Y 30/00

(57) ABSTRACT

This disclosure relates to compounds, pharmaceutical compositions comprising them, and methods of using the compounds and compositions for treating apicomplexan parasite infections. More particularly, this disclosure relates to tetrahydroquinolinone compounds and pharmaceutical compositions thereof, methods of selectively inhibiting cytochrome b with these compounds, and methods of treating diseases that benefit from selective cytochrome b inhibition, such as a T. gondii infection.

### CROSS-REFERENCE TO RELATED APPLICATIONS

This application claims the benefit of priority of U.S. Provisional Patent Application No. 63/026,415, filed May 18, 2020, all of which is incorporated by reference in its entirety.

### BACKGROUND OF DISCLOSURE

### Field of Disclosure

This disclosure relates to compounds, pharmaceutical compositions comprising them, and methods of using the compounds and compositions for treating apicomplexan parasite infections. More particularly, this disclosure relates to tetrahydroquinolinone compounds and pharmaceutical compositions thereof, methods of selectively inhibiting cytochrome b with these compounds, and methods of treating diseases that benefit from selective cytochrome b inhibition, such as a T. gondii infection.

### Technical Background

Malaria results in the death of about 0.5 million children a year, with drug resistance affecting the usefulness of successive generations of new medicines. The related apicomplexan parasite, Toxoplasma gondii, is the most frequent parasitic infection of humans in the world. It plays a significant role in food-born associated death in the USA, destruction of the human retina, and death and illness from recrudescent disease in the immune compromised or immunologically immature. It has been estimated that there are 1.9 million new cases of this congenital T. gondii infection globally over a ten-year period, causing 12 million disability adjusted life years from damage to the fetal brain and eye. Toxoplasmosis is an often neglected, untreated or mistreated disease. There are approximately 2 billion people throughout the world who have this parasite in their brain lifelong, some with known, severe, adverse consequences. There are possible additional, harmful effects for a substantial number of chronically infected people as this parasite modulates signature pathways of neurodegeneration, motor diseases, epilepsy, and malignancies. No medicine eliminates this chronic, encysted form of the parasite. New and improved medicines are greatly needed to cure Toxoplasma and Plasmodia infections. These parasites often share the same molecular targets for medicines due to a relatively close, apicomplexan, phylogenetic relationship. Thus, medicine development for each of these parasites can inform development of medicines that benefit treating the other.

One such shared molecular target is the mitochondrial cytochrome bc1 complex that is important for the survival of apicomplexan parasites such as Plasmodia and T gondii. Cytochrome b is a subunit of the cytochrome bc, complex, an inner mitochondrial membrane protein that is part of the electron transport chain. Activity of this complex is integral to oxidative phosphorylation and generation of ATP. Cytochrome b activity appears to be necessary for the replication and persistence of the parasite, and is the site of action of atovaquone. Cytochrome b is the target for quinolone-based compounds, but significant problems with solubility and toxicity have been noted with known cytochrome b inhibitors. Therefore, there remains a need for effective cytochrome b inhibitors that do not have the drawbacks of the currently known cytochrome b inhibitors.

### SUMMARY OF DISCLOSURE

The present disclosure is related to novel quinolone-like inhibitors, tetrahydroquinolinones (THQs). Thus, in one aspect, the disclosure provides compounds selected from:

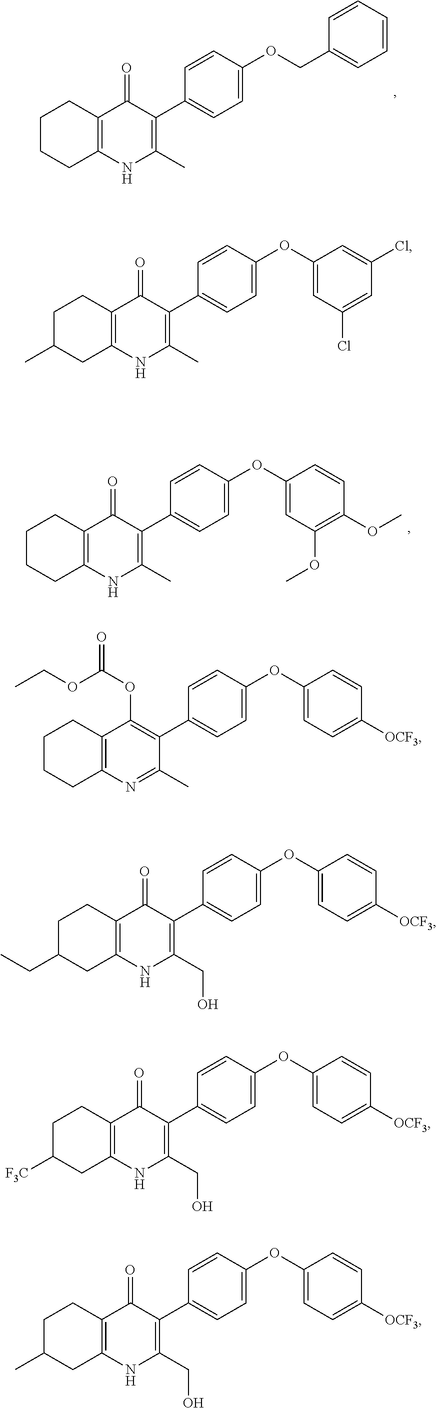

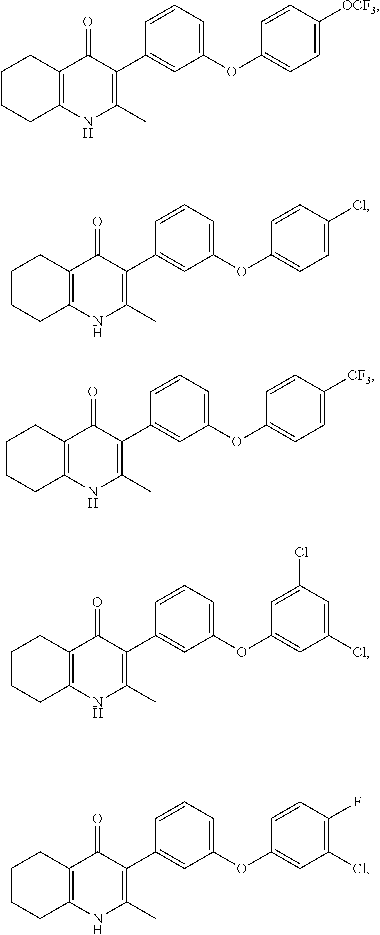 [SMILES: Cc1[nH]c2c(c(=O)c1-c1cccc(Oc3ccc(OC(F)(F)F)cc3)c1)CCCC2 ; Cc1[nH]c2c(c(=O)c1-c1cccc(Oc3ccc(Cl)cc3)c1)CCCC2 ; Cc1[nH]c2c(c(=O)c1-c1cccc(Oc3ccc(C(F)(F)F)cc3)c1)CCCC2 ; Cc1[nH]c2c(c(=O)c1-c1cccc(Oc3cc(Cl)cc(Cl)c3)c1)CCCC2 ; Cc1[nH]c2c(c(=O)c1-c1cccc(Oc3ccc(F)c(Cl)c3)c1)CCCC2]

or a pharmaceutically acceptable salt thereof.

Another aspect of the disclosure provides a pharmaceutical composition including one or more compounds of the disclosure as described herein (e.g., compounds provided above) and a pharmaceutically acceptable carrier, solvent, adjuvant or diluent.

Another aspect of the disclosure provides nanoparticle formulations comprising one or more tetrahydroquinolinones as disclosed herein. In one embodiment, the disclosure provides a nanoparticle formulation including:
an aqueous carrier fluid; and
a dispersion of particles within the aqueous carrier fluid, wherein the particles comprise a hydrophobic material with a surfactant and one or more compounds of the disclosure.
   In certain embodiments, such compounds are of formula (I),

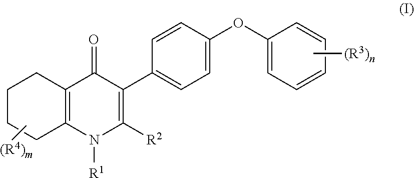

or a pharmaceutically acceptable salt thereof, wherein
m is 0 or 1;
n is 0, 1, or 2;
R¹ is hydrogen or C₁-C₃ alkyl;
R² is hydrogen, C₁-C₃ alkyl, or —CH₂OH;
each R³ is independently halogen, C₁-C₃ alkyl, C₁-C₃ haloalkyl, C₁-C₃ alkoxy, C₁-C₃ haloalkoxy; and
each R⁴ is independently C₁-C₃ alkyl, or C₁-C₃ haloalkyl.
   In certain embodiments, such compounds are listed in Table 1. In certain embodiments, such compounds are as disclosed in International Patent Publication WO 2017/112678, published 29 Jun. 2017, incorporated herein by reference in its entirety (such as the compounds disclosed in the table at pages 141-145, and claims 20 and 21).

Another aspect of the disclosure provides methods for treating an apicomplexan parasite infection. In certain embodiments, such methods include administering to a subject in need thereof an amount effective to treat the infection of one or more of modulators of one or more of the genes listed in FIG. 6A or Table 3 (in FIG. 8). In certain embodiments, such methods include administering to a subject in need thereof an amount effective to treat the infection (i) one or more of eukaryotic initiation factor-2a kinase (eif2k) inhibitors selected from the group consisting of anti-eif2k antibody, anti-eif2k aptamer, eif2k small interfering RNA, eif2k small internally segmented interfering RNA, eif2k short hairpin RNA, eif2k microRNA, and eif2k antisense oligonucleotides, and (ii) one or more compounds as described here, such as the compounds of formula (I) as described in any of claims 3-5, or compounds listed in Table 1, or compounds as disclosed in International Patent Publication WO 2017/112678 as noted above.

As used, modulators are any moiety that can increase or decrease and functional characteristics of the gene or gene product (RNA or protein), including but not limited to expression, stability, activity, or other characteristics of the gene or gene product (RNA or protein).

In certain embodiments, the apicomplexan parasite infection is a T. gondii infection.

Another aspect of the disclosure method for identifying test compounds for apicomplexan parasite therapy. Such methods include identifying one or more of test compounds that modulate activity of one or more of the genes listed in FIG. 6A or Table 3 (in FIG. 8).

Another aspect of the disclosure method for diagnosing an apicomplexan parasite infection (such as a T. gondii infection). Such methods include:
(a) determining an expression level of one or more of the up-regulated and/or down-regulated genes listed in FIG. 6A or Table 3 (in FIG. 8) in a biological sample from a subject; and
(b) identifying a subject as having an apicomplexan parasite infection if subject has:
   (i) an expression level of 1, 2, 3, 4, 5, or more up-regulated genes increased relative to a threshold,
   (ii) an activity level of protein expressed from 1, 2, 3, 4, 5, or more up-regulated genes increased relative to a threshold,
   (iii) an expression level of 1, 2, 3, 4, 5, or more down-regulated genes decreased relative to a threshold, and/or
   (iv) an activity level of protein expressed from 1, 2, 3, 4, 5, or more down-regulated genes decreased relative to a threshold.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the systems and methods of the disclosure, and are incorporated in and constitute a part of this specification. The drawings illustrate one or more embodiment(s) of the disclosure and, together with the description, serve to explain the principles and operation of the disclosure.

FIG. 1 shows characteristics Structure Activity Relationship (SAR) analysis. The SAR compared the effects of changing R¹ as 7-Et, 7-Me, 6-CF₃, or 6-Me on activity against T.gondii RH strain tachyzoites, solubility, and stability. Colour Key in B: Activity. Green<50 nM, Red>1 μM; Solubility in 100 mM Phosphate Buffer (pH 7.4): Amber>10 μM, Red<10 μM; Metabolic Stability Green>120 minutes, Amber 60-120 mins, red<60 minutes. SAR panel displays only representative structures and trends within the JAG compound series. JAG21 is highly active, has the longest predicted half-life for humans of initial compounds tested (green), combined with improved solubility, no hERG liability, and predicted capacity to cross the blood brain barrier (BBB). Definitions of ADMET terminology are in the Materials and Methods. In summary, in the SAR overall, nitrogen atoms not tolerated in aryl ring (C) and the 4-position was optimal for phenol substituent.

FIG. 2 illustrates JAG21 is potent in vitro against T. gondii, tachyzoites and bradyzoites, and multiple drug resistant strains of P. falciparum. A. JAG21 is effective Against RH-YFP tachyzoites, and does not harm human cells. Potent effect of JAG 50 is also shown. A representative experiment is shown. N=triplicate wells in at least two biological replicate experiments. Relative fluorescence units are shown on vertical axis, where decrease in fluorescence compared to diluent DMSO in media control indicates parasite inhibition. Horizontal axis indicates different treatment conditions: This shows results of testing of fibroblasts in media (HFF), DMSO control, positive control pyrimethamine and sulfadiazine (P/S), and concentrations of JAG21 and JAG50 utilized. Differences were not statistically significant in the cytotoxicity WST assay measuring toxicity against host cells (data not shown). B. JAG21 is effective against EGS bradyzoites. Effect of JAG21 in reducing bradyzoites in HFF by parasite strain EGS. HFF were infected by EGS and treated with JAG21 at concentrations indicated. Slides were stained with Dolichos Biflorus Agglutinin conjugated with FITC (which stains the cyst wall) and DAPI, and observed with fluorescence microscopy. The red arrows point to the Dolichos enclosed organisms formed in tissue culture. These were eliminated with treatment with JAG21. This experiment was performed>4 times. These experiments were performed with three different observers reviewing slides at the microscope quantitating fields for each condition. Slides were also scanned and the scans of the slides were reviewed so all fields in the entire slide were noted to be consistent. C. Synergy of JAG21 and atovaquone against Rh-YFP tachyzoites in vitro. Isobologram comparing JAG21, atovoquone, and JAG21 plus atovaquone demonstrates synergy. D. THQs effective against drug resistant P. falciparum. Dose-response phenotypes of a panel of P. falciparum parasite lines. IC₅₀ values were calculated using whole-cell SYBR Green assay and listed as mean±standard deviation of three biological replicates, each with triplicate measurements. The D6 strain is a drug sensitive strain from Sierra Leone, the TM91-C235 strain is a multi-drug resistant strain from Thailand, the W2 strain is a chloroquine resistant strain from Thailand, and the C2B strain is a multi-drug resistant strain with resistance against atovaquone. E. Solubility and Stability in human and mouse liver microsomes comparing MJM 170, JAG21 and JAG50. F. JAG21 CYP450 Inhibition, CACO-2, hERG, PPB, BBB (MDCK-MDK1) efflux analyses.

FIG. 3 shows effect of JAG21 and other THQ compounds on mitochondrial functions of T.gondii and. P. falciparum and HFF-hTERT A. Maximum mitochondrial membrane depolarization of JAG21, JAG39, JAG46, JAG47, JAG50 and atovaquone (4 μM) and FCCP (5 μM) on T. gondii after digitonin was added where indicated by the arrow to permeabilize cells and permit a necessary mitochondrial substrate (succinate) to reach intracellular organelles. The addition of the indicated compounds is shown by the second arrow. B. Quantification of the depolarization shown in A. The relative depolarization of each compound was normalized to the depolarization by FCCP, which was considered 100% depolarization. C. Effect of various concentrations of JAG21 and Atovaquone on the mitochondrial membrane potential on T. gondii measured as in A. The first arrow indicates digitonin addition and the second arrow indicates the addition of compounds at the specified concentration. D. Quantification of the depolarization of mitochondrial membrane potential on T. gondii measured in C. The relative depolarization of each compound was normalized to the depolarization by FCCP (100%). E. Mitochondrial membrane depolarization of HFF-hTERT in suspension by JAG21 and atovaquone. The first arrow indicated the addition of digitonin, and the second arrow indicates addition of the indicated compounds at the indicated concentration. F. Quantification of the depolarization measured in E. The relative depolarization of each compound was normalized with the depolarization by FCCP, which was considered 100%. B, D, E X±S.D., N=3 independent experiments. Statistical analysis (unpaired student t test) was performed using GraphPad Prism 8.0 (GraphPad Software, Inc., San Diego, Calif.). **. P<0.01. ***. P<0.001. g. JAG21, JAG99 and MJM210 (1 μM) inhibited P. falciparum cytochrome c reduction. Vehicle (DMSO)/atovaquone (1 μM) were negative/positive controls, 1290 respectively. X±S.D., N=4 independent experiments.

FIG. 4 illustrates binding studies of JAG021 to bovine bc1. A. Bovine Cytbc1 activity assays showing 36% and 63% inhibition at 0.1 and 1 μM concentration of JAG021, respectively. N=at least two biological replicate experiments with similar results. B. The Cytbc1 structure presented in cartoon style with clear omit (Fo-Fc) electron density map for the bound JAG021 compound only in the Qᵢ site showing selectivity within the binding pocket. Qᵢ and Qₒ sites are marked by black ellipsoids. C. The bound JAG021 compound (orange) within the Q, site with corresponding (2Fo-Fc) electron density map contoured at 1 a level as grey mesh. The residues that make close interactions with the bound inhibitor are shown in stick format and labelled. D. 2D pharmacophore analysis of JAG021 binding pocket produced using Ligplot+LS-2011. Hydrophobic interactions are shown as red spikes, hydrogen bond with Ser35 is shown by green dashes. E. Cryo-EM derived structure of the Cytbc1 bound JAG021 structure with corresponding density map contoured at 3 a level suggesting two different positions for the head group represented by two regions of density shown as yellow mesh. The Cytbc1 structure bound to the pyridone GSK932121 (PDB:4D6U) (F) and quinolone MJM170 (PDB:5NMI) (G) in the Qᵢ site. Haem and compounds are shown as colored sticks, Fe ion as orange sphere and hydrogen bonding as black lines. Hydrogen bonding with Ser35 is shown as black dashes.

FIG. 5 illustrates JAG21 is a mature lead that protects against T. gondii and P.berghei in vivo. A. JAG21 treatment for 14 days protects against T.gondii tachyzoites in vivo. Tachyzoite challenge with Prugneaud luciferase parasites imaged with leuciferin using IVIS demonstrates that treatment with JAG21 eliminates leuciferase-expressing parasites and leads to 100% survival of JAG21 treated infected mice. No cysts were found in brains of mice at 30 days after infection when they have been treated with JAG21 for the first 14 days after infection. There were 2 biological replicate experiments with 5 mice per group with similar results. B. JAG21 and JAG21 plus tafenoquine markedly reduce Me49 strain brain cyst numbers in vivo in Balb/C mice at 30 days after infection. Parasites were quantitated by scanning the entire immunoperoxidase stained slide in an automated manner and by two observers blinded to the experimental treatment using microscopic evaluation. In each of two experiments, the numbers of mice per group were as follows: Experiment 1 had 4 diluent controls, 5 JAG21, 4 JAG21/Tafenoquine treated mice; and Experiment 2 had 5 diluent controls, 5 JAG21, 3 JAG21/Tafenoquine treated mice. Immunoperoxidase staining was performed. Parasite burden was quantitated using a positive pixel count algorithm of Aperio ImageScope software. Positive pixels were normalized to tissue area (mm²). Quantification was by counting positive pixels per square area. The entire brain in one section was scanned for each mouse. The parasite burden was quantitated as units of positive pixels per mm². The average±S.E.M. numbers of mm² per slide quantitated was 30.2±1.6 mm² per mouse for this quantification. Each high power field of view shown in C is approximately 0.02 mm² per field of view. A representative single experiment is presented and the data from the two experiments analyzed together also demonstrated significant differences between the untreatated and treated groups (p<0.01; FIG. 9). C. Microscopic evaluation of the slides reveal effect of JAG21 and JAG21 plus tafenoquine having the same pattern as the automated quantitation of immunoperoxidase stained material. There are usual appearing cysts in the DMSO control untreated mice as shown in the top panels, and rare cysts in the treated mice with most of the brown material appearing amorphous (bottom panels). D. JAG21 nanoformulation dosages administered to P.berghei infected C57B16/albino mice compared with vehicle control. Design of single dose and 3 day dose experiments. E. JAG21 nanoformulation cures P. berghei sporozoites (left panel), blood (middle panel) and liver stages (right panel) with oral administration of a single dose of 2.5 mg/kg or 3 doses at 0.625 mg/kg. Single dose causal prophylaxis in 5 C57BL/6 albino mice at 2.5 mpk dosed on day 0, 1 hour after intravenous administration of 10,000 P. berghei sporozoites. Shown is 3 dose causal prophylaxis treatment in 5 C57BL/6 albino mice at 0.625 mpk dosed on days −1, 0, and +1. Representative figure showing survival, luminescence and parasitemia quantitated by flow cytometry for 5 mg/kg.

FIG. 6 shows T. gondii ΔRPS13 transcriptome during Primary Human Brain Neuronal Stem Cell (NSC) infection and in-vivo susceptibility to JAG21 and TAF treatment are reminiscent of literature findings with malaria hypnozoites. A, P. cynomolgi-T. gondii best reciprocal match genes significantly upregulated (red) or downregulated (blue) in P. cynomolgi hypnozoites compared to liver-schizont stage and in ΔRPS13 after downregulation of rps13 gene expression (p-value s 0.05, FDR s 0.2). B, Gene-set enrichment analysis of ΔRPS13±Tc. Blue and red nodes denote gene-sets enriched in presence or absence of Tc respectively. Node diameters are proportional to number of genes belonging to corresponding gene-sets. Edge thickness is proportional to number of genes shared between connected nodes. C, Survival rate of mice infected with 100,000 ΔRPS13 followed by treatment with diluent, JAG21, tafenoquine (TAF) or the two together (JAG21/TAF). Then tetracycline was added at 14 days. The combination of the two compounds resulted in improved time of survival (p<0.03, Experiment 1; p=0.08 Experiment 2, p=0.002 Experiment 1+2). The full data are presented in the box below the image in FIG. 6C. In 6C, Rx refers to treatment of mice with diluent (DMSO), Tafenoquine (TAF), or JAG21, or JAG21 and TAF. ΔRPS13 is referred to as RhRPS13Δ in the title of the box in FIG. 6C. In FIG. 10, histological preparations that are immunoperoxidase stained for T gondii antigens from a pilot study were prepared (FIG. 10). These are images, in FIG. 10 of liver and spleen from IFN γ receptor knock out mice without treatment on days 7 and 14 after infection. In those mice without any treatment there was amorphous brown immunoperoxidase stained material in FIG. 10A. When tetracycline (aTet) was administered on day 14 after infection in drinking water on day 14 and tissues obtained and immunostained for T. gondii antigens from mice that died or became very ill, organisms clearly recognizable could be seen (FIG. 10B-E). Design of the treatment experiment with control DMSO diluent, JAG21 alone, Tafenoquine alone (TAF) or the two together (JAG/TAF) with full data for each of the groups and with the composite analysis from replicate experiments, including numbers of mice, are shown in FIG. 10C, D. FIG. 10C, D shows prolongation of survival time, but there is not durable protection against ΔRPS13 in these immune compromised mice treated with JAG21/TAF as described. This is summarized in C to demonstrate early prolongation of survival time with the detailed data in FIG. 10. D. Gene ontology enrichment analysis of ΔRPS13±Tc. Node and edge conventions are the same as in B. There were at least 2 biologic replicates of each experiment.

FIG. 7 shows oral nanoformulation of JAG21 potently protects against 2000 highly virulent RH strain tachyzoites given intraperitoneally. A. Following sonication produces nanoparticles of about 2.86 μM. B. Single oral dose of 10 mg/kg reduced intraperitoneal tachyzoites measured by RH YFP expression and counting with hematocytometer (p<0.03). C. Three daily 10 mg/kg doses markedly and significantly reduces intraperitoneal parasite burden measured as fluorescence and by hematocytometer on the fifth day (p<0.001). No compound was administered after the third day. N=at least 2 biological replicate experiments with 5 mice per group with similar results.

FIG. 8 provides Table 3, which is to accompany FIG. 6.

FIG. 9 illustrates JAG21 reduced immunostained material with or without Tafenoquine. Results were similar when cysts were quantitated microscopically. This shows pooled results of two replicate trials.

FIG. 10 illustrates RPS13D in IFNγ receptor knockout mice. RPS13D in IFNγ receptor knockout mice created amorphous immunoperoxidase stained material in spleen and liver 7 and 14 days after infection when no tetracycline was given. The brown material looked less recognizable as T.gondii at 14, than at 7 days after initiation of infection. A. 7 days and 14 days after infection. B. When aTet is given. When a Tet was given to these mice in their drinking water after 7 or 14 days, parasites replicated and were lethal for ˜50-80% of the mice. C,D. Design of experiment with treatment and primary data and Kaplan Meier analysis for this experiment is shown. When similarly infected mice were first treated on day −1 with tafenoquine, and for the first 14 days with JAG21 with each compound alone or the two together, it was found that the combination of JAG21 and tafenoquine prolonged survival modestly, although ultimately mice in that group also died (C,D). The pale yellow beige shows initial enhanced survival suggesting that the combination was modestly better than each was alone, as shown in FIG. 6C. A, B, demonstrates that G1 arrested organisms can persist in immune incompetent mice and although even with immunoperoxidase staining, organisms may not be easily identified morphologically and can recrudesce. C,D suggested that dosing in those with immune compromise may need to be optimized and possibly continued for the duration of the immune compromise. E. Immunostaining after aTet given in a tafenoquine treated mouse demonstrated multiple single organisms at time of death of the mouse. C,D,E, demonstrate that adding tafenoquine to JAG21 prolongs survival modestly but as dosed did not result in durable protection after.

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and systems described herein can be configured by the person of ordinary skill in the art to meet the desired need. As provided above, apicomplexan infections cause substantial morbidity and mortality, worldwide presenting urgent need for new and improved therapies. A markedly increased expression of cytochrome b in the currently untreatable T. gondii bradyzoite life-cycle stage was recently found (McPhillie et al. 2016). Thus, there is a great need for a compound that would inhibit tachyzoites, bradyzoites and three life cycle stages of even drug-resistant Plasmodia was developed, without a need for a pro-drug as has been needed in other attempts to target apicomplexan cytochrome b. The disclosure aimed to improve upon the physicochemical properties of napthoquinones and endochin-like quinolones (ELQs) targeting cytochrome b, including poor aqueous solubility and toxicity.

The disclosure provides novel quinolone-like inhibitors, THQs, with improved solubility, and lower toxicity, compared to known compounds in the literature. Without being bound by a theory, it is reasoned that the increased ‘sp3’ character of the THQs (i.e., moving from rod-like to sphere-like 3D space) could provide the required improvement in solubility that would allow for optimal pharmacokinetic properties. Molecules with an increased percentage of ‘sp3 character’ tend to be more three-dimensional than their planar (‘sp2-rich’) counterparts. The terms ‘sp2’ and ‘sp3’ refer to the shape of their hybridized atomic orbitals, which have trigonal planar and tetrahedral geometries respectively. Flat aromatic rings (‘sp2-rich’) are ubiquitous in drug discovery campaigns, but molecules with more ‘sp3 character’ are often more specific for their protein target and can have better physicochemical properties. Further, without being bound by a theory, it is reasoned that the larger binding pocket in the parasite enzymes, compared to their mammalian counterparts, would provide room for bulkier substituents to minimize effect on the human enzyme.

The disclosure in certain embodiments provides a preclinical lead candidate based on potent and selective inhibition of P. falciparum and P. berghei and T. gondii cytochrome bc1 for the treatment of malaria and toxoplasmosis. The candidate compound demonstrates high efficacy in relevant in vitro and in vivo models of the diseases, and has considerable potential for broad-spectrum use (i.e., against T. gondii tachyzoites and encysted bradyzoites and drug resistant Plasmodia). For example, in certain embodiments, the disclosure provides a next generation anti-apicomplexan lead compound, JAG21, a tetrahydroquinolone, with increased sp3-character to improve parasite selectivity. Relative to other cytochrome b inhibitors, JAG21 has improved solubility and ADMET properties, without need for pro-drug. JAG21 significantly reduces Toxoplasma gondii tachyzoites and encysted bradyzoites in vitro, and in primary and established chronic murine infections. Moreover, JAG21 treatment leads to 100% survival. Further, JAG21 is efficacious against drug-resistant Plasmodium falciparum in vitro. Causal prophylaxis and radical cure are achieved after P. berghei sporozoite infection with oral administration of a single dose (2.5 mg/kg) or three days treatment at reduced dose (0.625 mg/kg/day), eliminating parasitemia and leading to 100% survival. Enzymatic, binding, and co-crystallography/pharmacophore studies demonstrate selectivity for apicomplexan relative to mammalian enzymes. JAG21 has significant promise as a pre-clinical candidate for prevention, treatment and cure of toxoplasmosis and malaria.

The present disclosure is related to novel quinolone-like inhibitors, tetrahydroquinolinones (THQs) as provided above, or a pharmaceutically acceptable salt thereof.

Another aspect of the disclosure provides nanoparticle formulations comprising one or more tetrahydroquinolinones as disclosed herein. In one embodiment, the disclosure provides a nanoparticle formulation including:
an aqueous carrier fluid; and
a dispersion of particles within the aqueous carrier fluid, wherein the particles comprise a hydrophobic material with a surfactant and one or more compounds of the disclosure.

In certain embodiments, the particle comprises one or more compounds of formula (I),

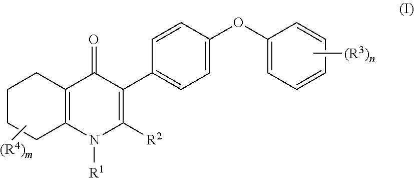

or a pharmaceutically acceptable salt thereof, wherein
m is 0 or 1;
n is 0, 1, or 2;
R¹ is hydrogen or C₁-C₃ alkyl;
R² is hydrogen, C₁-C₃ alkyl, or —CH₂OH;
each R³ is independently halogen, C₁-C₃ alkyl, C₁-C₃ haloalkyl, C₁-C₃ alkoxy, C1-C₃ haloalkoxy; and
each R⁴ is independently C₁-C₃ alkyl, or C₁-C₃ haloalkyl.

In certain embodiments, m is 0 or 1. In certain embodiments, m is 0.

In certain embodiments, n is 0, 1, or 2. In certain embodiments, n is 0 or 1.

In certain embodiments, R¹ is hydrogen, methyl, or ethyl. In certain embodiments, R¹ is hydrogen or methyl. In certain embodiments, R¹ is hydrogen.

In certain embodiments, R² is hydrogen, methyl, ethyl, or —CH₂OH. In certain embodiments, R² is hydrogen, methyl, or ethyl. In certain embodiments, R² is methyl, ethyl, or —CH₂OH. In certain embodiments, R² is methyl or —CH₂OH. In certain embodiments, R² is methyl.

In certain embodiments, each R³ is independently halogen, C₁-C₃ haloalkyl, C₁-C₃ alkoxy, or C₁-C₃ haloalkoxy. In certain embodiments, each R³ is independently halogen, methyl, trifluoromethyl, methoxy, or trifluoromethoxy. In certain embodiments, each R³ is independently halogen, trifluoromethyl, methoxy, or trifluoromethoxy.

In certain embodiments, each R³ is independently methyl, ethyl, or C₁-C₃ haloalkyl. In certain embodiments, each R⁴ is independently methyl, ethyl, or trifluoromethyl.

In an example embodiment, the compound of formula (I) is wherein
m is 0 or 1;
n is 0, 1, or 2;
R¹ is hydrogen;
R² is hydrogen, methyl, or —CH₂OH;
each R³ is independently halogen, trifluoromethyl, methoxy, or trifluoromethoxy; and
each R⁴ is independently methyl, ethyl, or trifluoromethyl.

In an example embodiment, the compound of formula (I) is JAG021.

In certain embodiments, the particle comprises one or more compounds that are listed in Table 1.

In certain embodiments, the particle comprises one or more compounds that are disclosed in International Patent Publication WO 2017/112678, published 29 Jun. 2017, and incorporated herein by reference in its entirety (such as the compounds disclosed in the table at pages 141-145, and claims 20 and 21).

As noted above, the nanoparticle formulation of the disclosure as provided herein includes a dispersion of particles within the aqueous carrier fluid, the particles comprising a hydrophobic material. In certain embodiments, the hydrophobic material is hydroxyethyl cellulose (HEC).

The hydrophobic material may be present in an amount sufficient to form the particle. For example, in certain embodiments, the hydrophobic material is present in an amount in a range of 3 mg/mL to 7 mg/mL. In certain embodiments, the hydrophobic material is present in an amount in a range of 4 mg/mL to 7 mg/mL. In certain embodiments, the hydrophobic material is present in an amount in a range of 3 mg/mL to 6 mg/mL. In certain embodiments, the hydrophobic material is present in an amount in a range of 4 mg/mL to 6 mg/mL. In certain embodiments, the hydrophobic material is present in an amount in a range of 3 mg/mL to 5 mg/mL. In certain embodiments, the hydrophobic material is present in an amount in a range of 5 mg/mL to 7 mg/mL. In certain embodiments, the hydrophobic material is present in an amount of about 5 mg/mL.

As noted above, the particle also comprises a surfactant. In certain embodiments, the surfactant is polyethylene glycol sorbitan monooleate (e.g., Tween® 80).

The surfactant may be present in an amount sufficient to form the particle. For example, in certain embodiments, the surfactant is present in an amount in a range of 1 mg/mL to 3 mg/mL. In certain embodiments, the surfactant is present in an amount in a range of 1.5 mg/mL to 3 mg/mL. In certain embodiments, the surfactant is present in an amount in a range of 1.5 mg/mL to 2.5 mg/mL. In certain embodiments, the surfactant is present in an amount in a range of 1 mg/mL to 2.5 mg/mL. In certain embodiments, the surfactant is present in an amount in a range of 1 mg/mL to 2 mg/mL. In certain embodiments, the surfactant is present in an amount in a range of 2 mg/mL to 3 mg/mL. In certain embodiments, the surfactant is present in an amount of about 2 mg/mL.

Another aspect of the disclosure provides methods for treating an apicomplexan parasite infection. In certain embodiments of the methods of the disclosure, the apicomplexan parasite infection is a T. gondii infection.

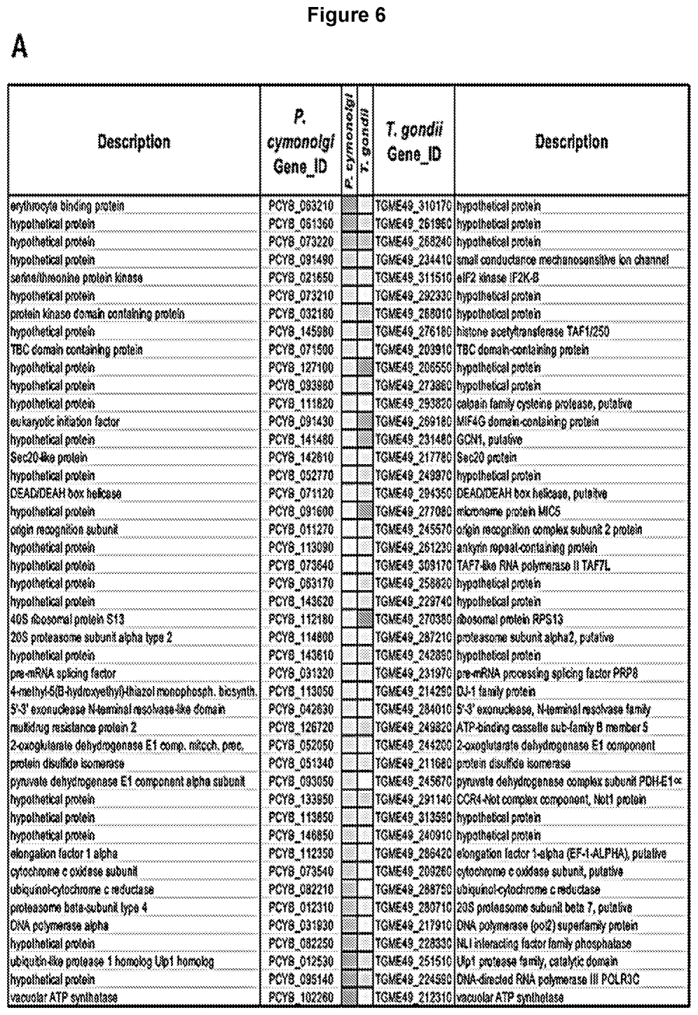

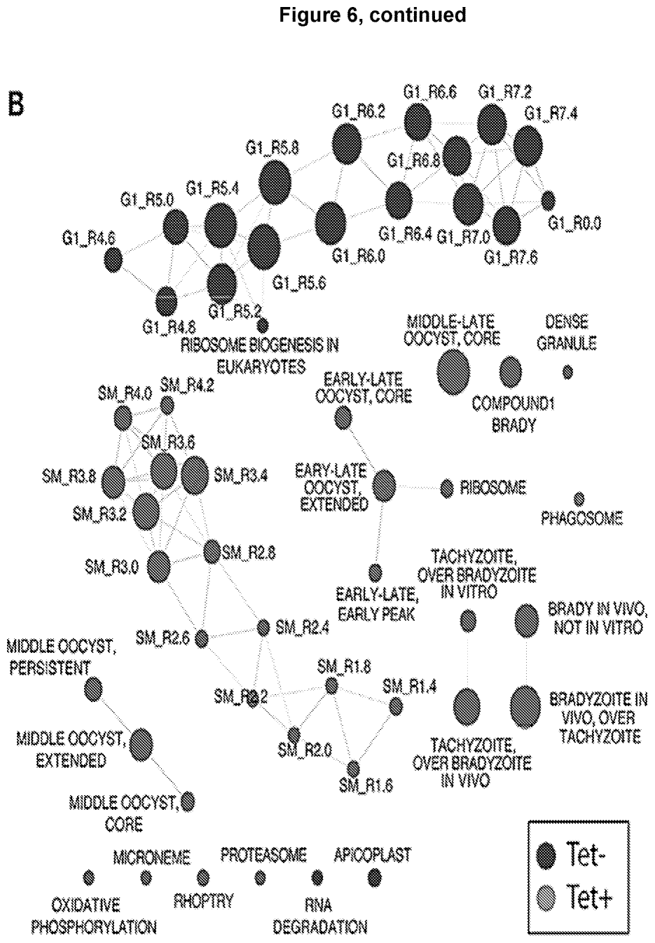

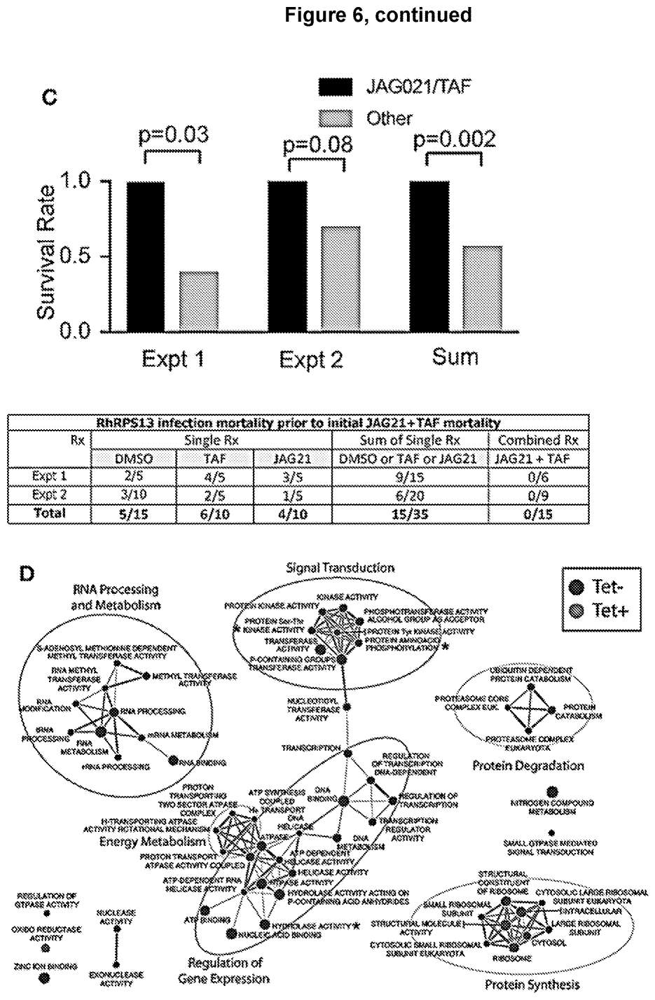

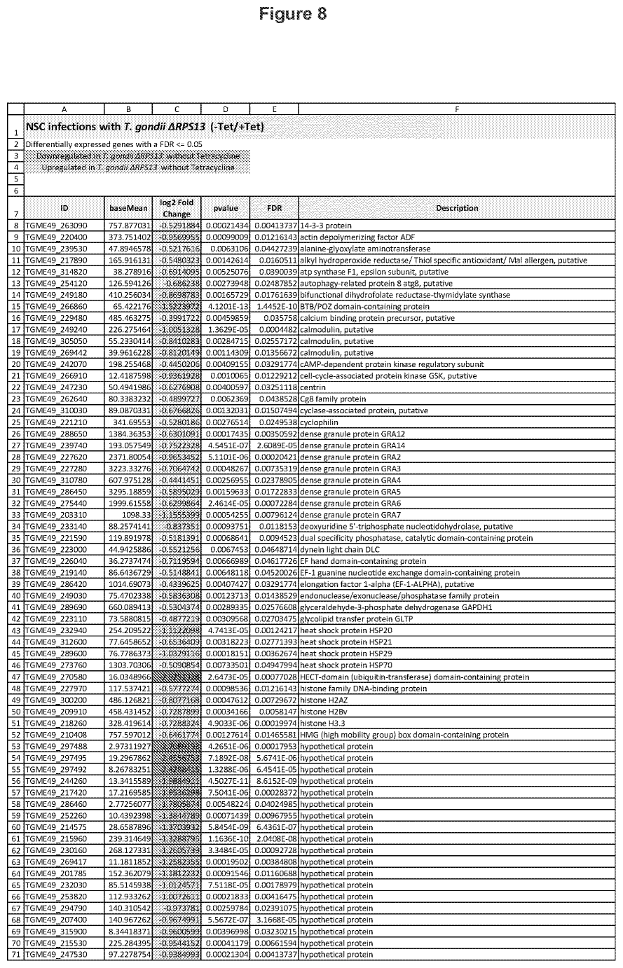

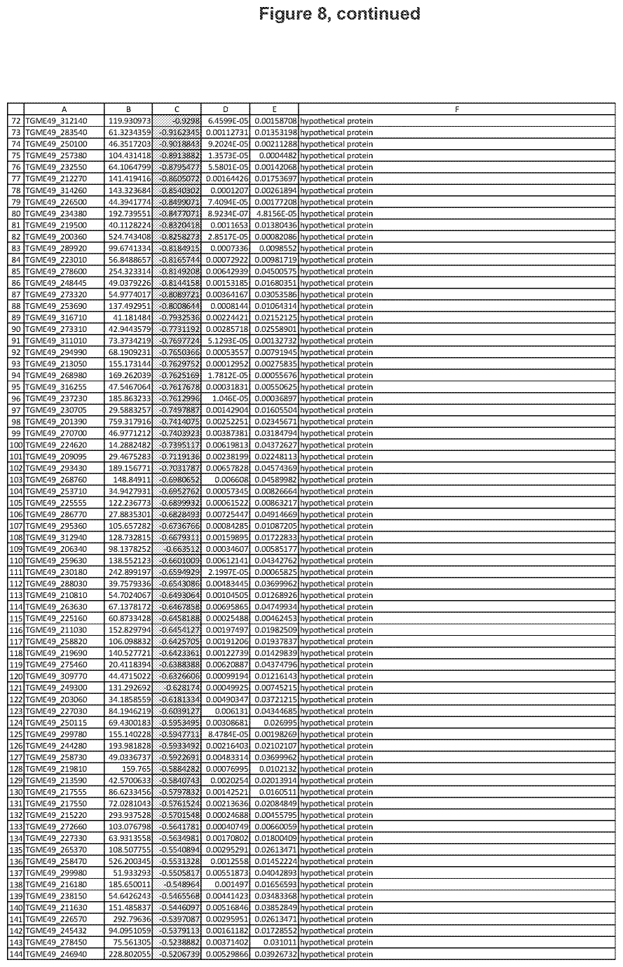

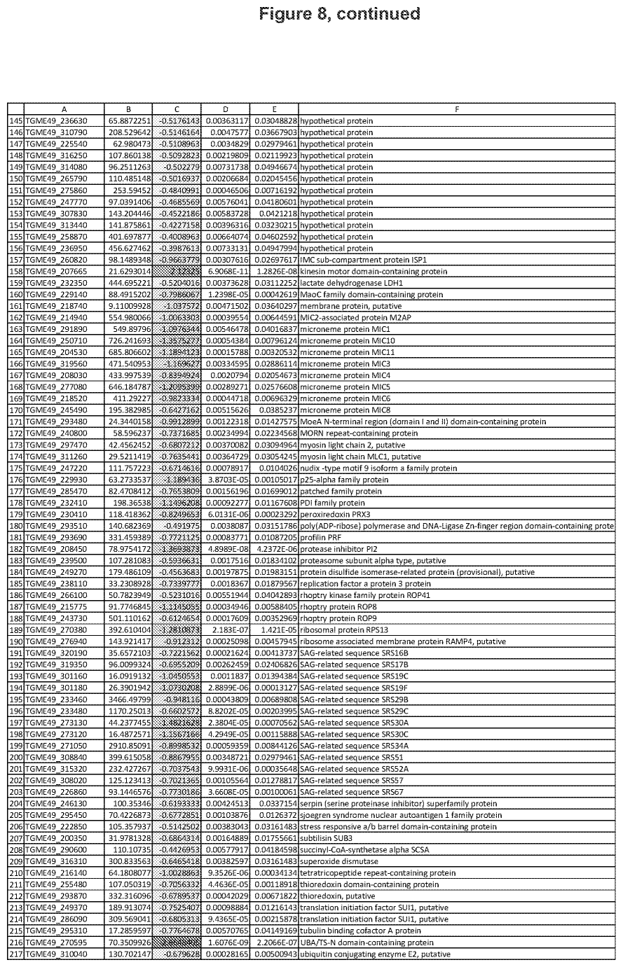

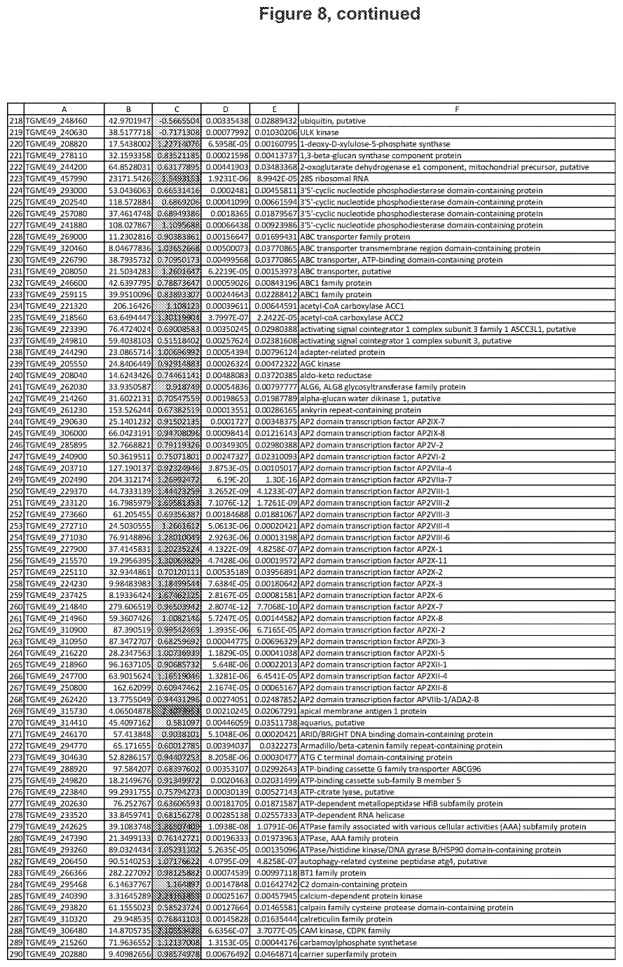

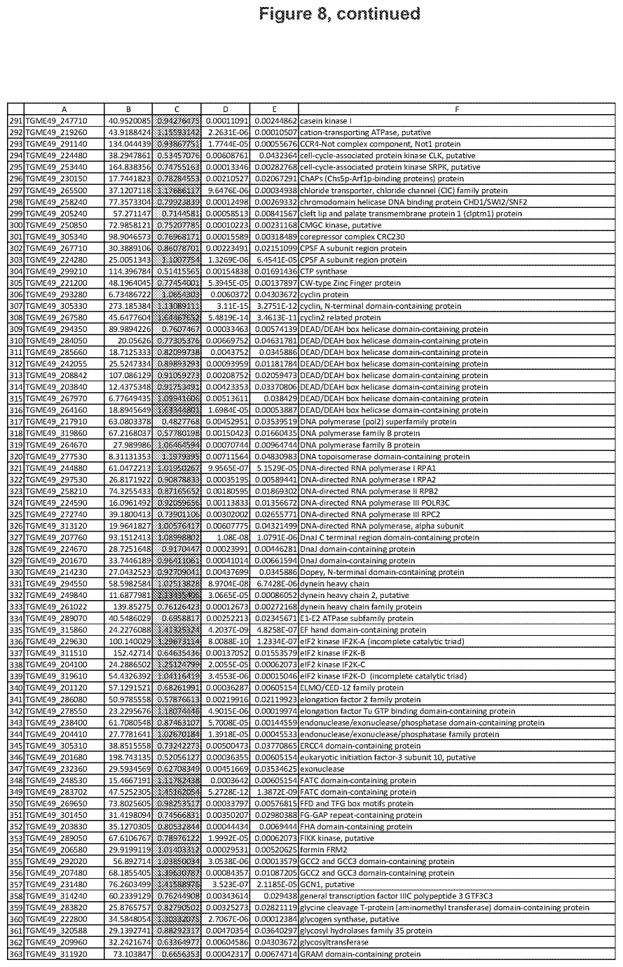

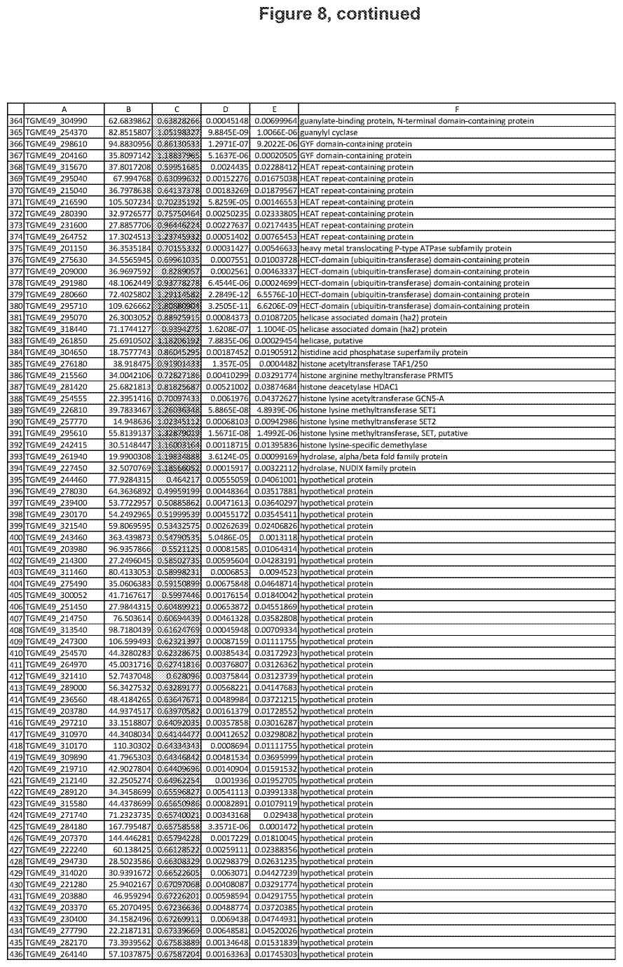

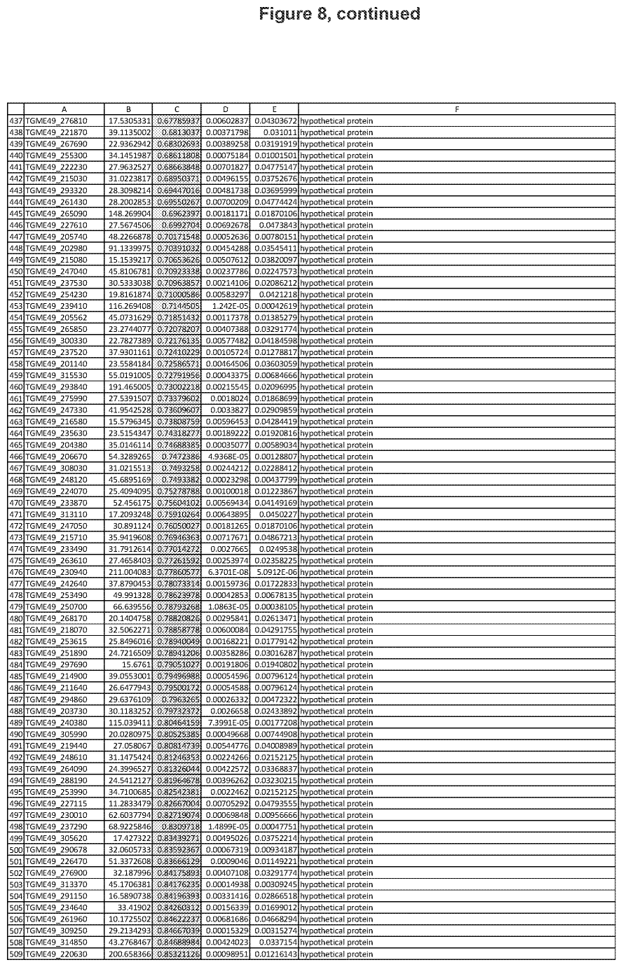

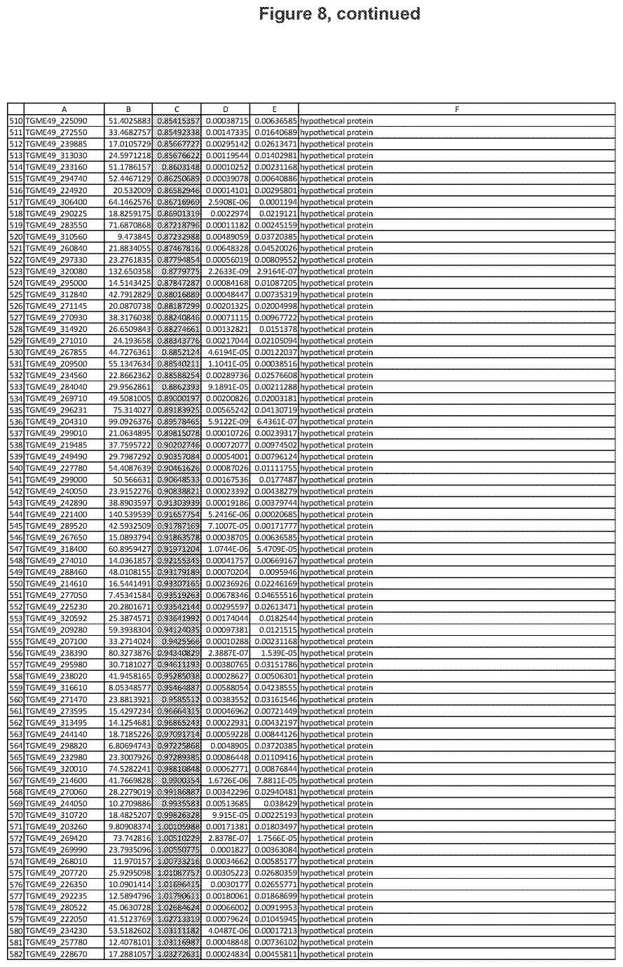

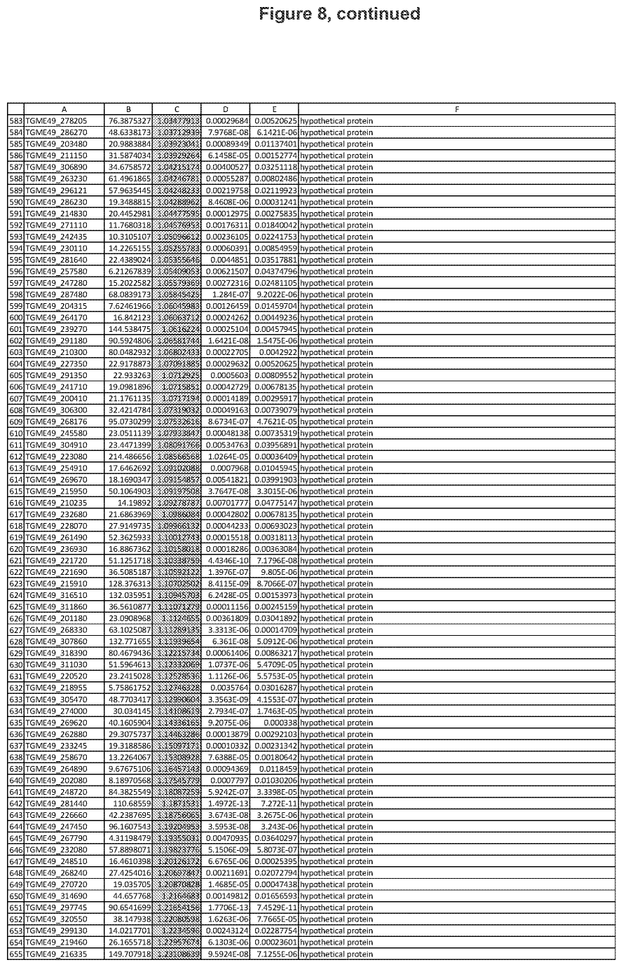

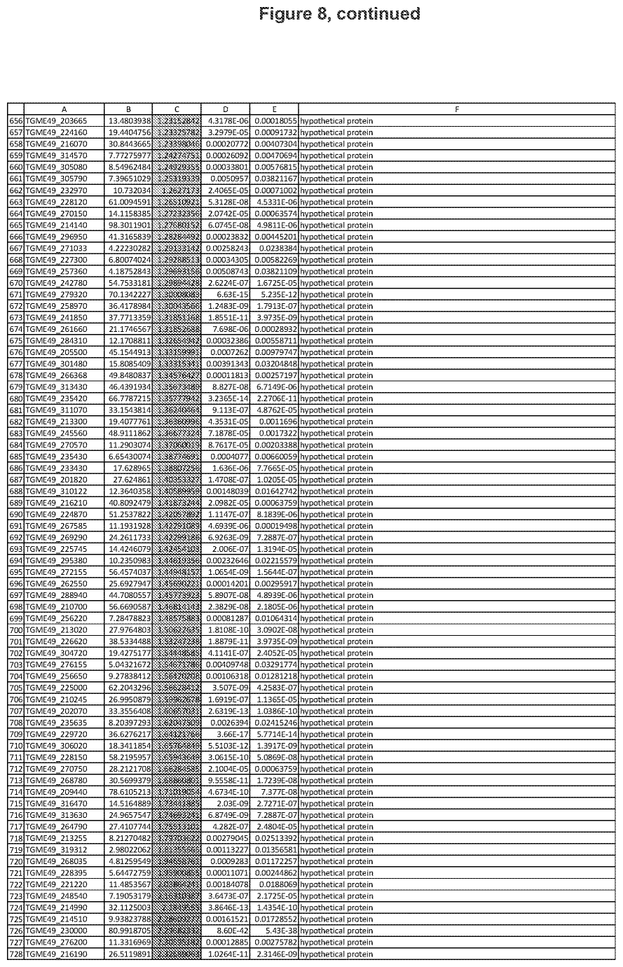

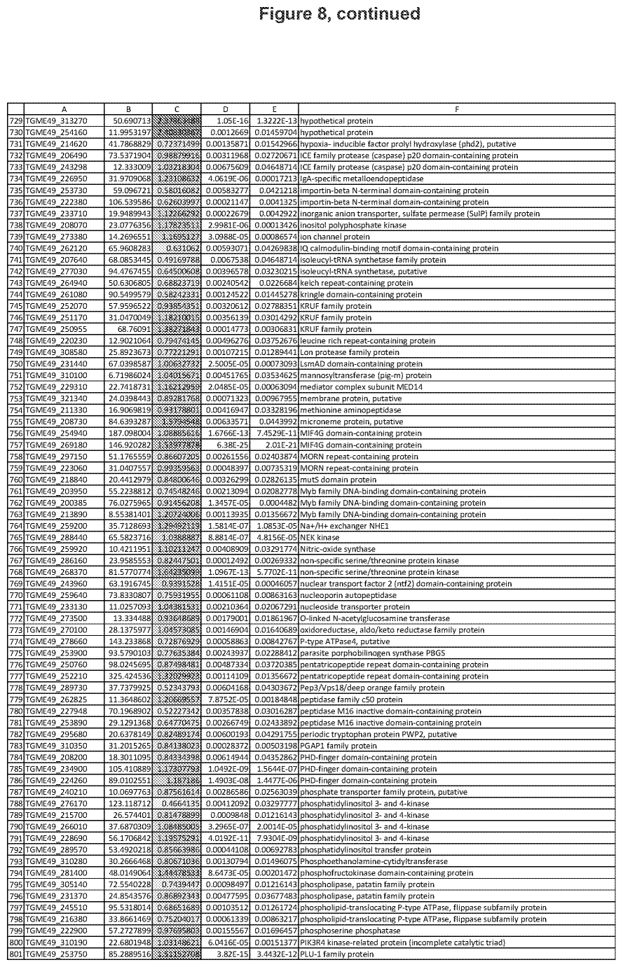

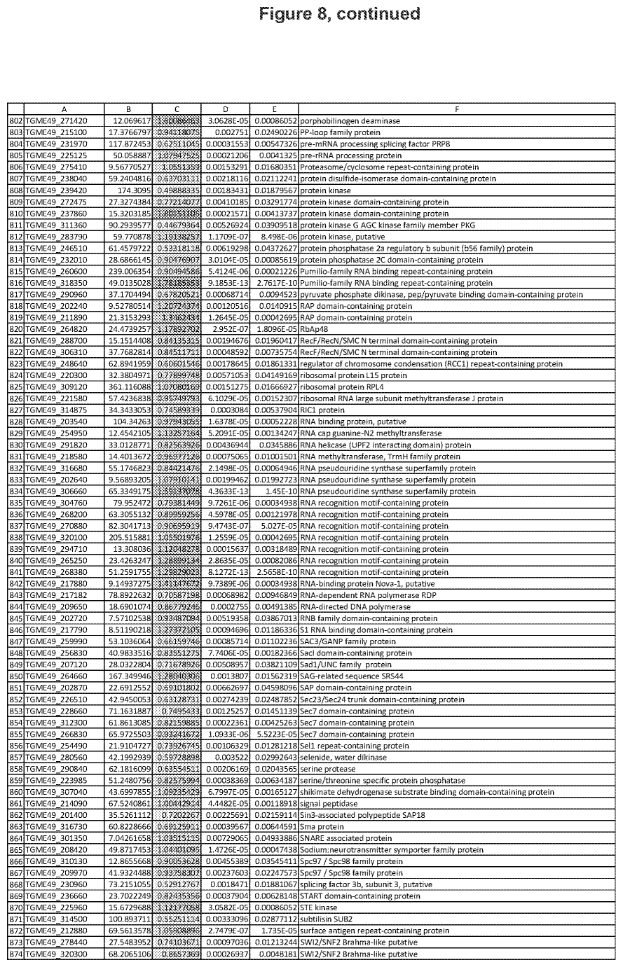

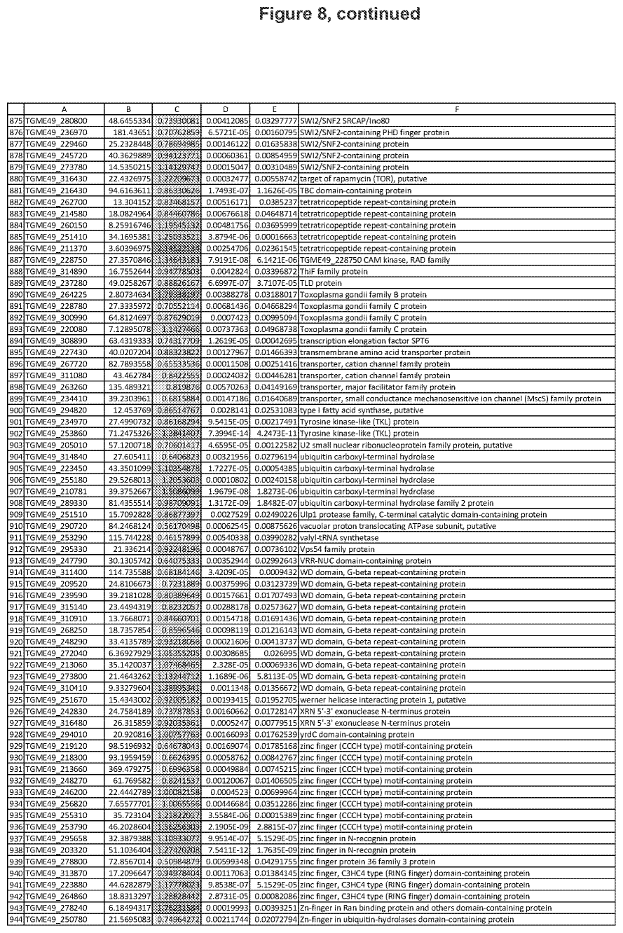

In certain embodiments, such methods include administering to a subject in need thereof an amount effective to treat the infection of one or more of modulators of one or more of the genes listed in FIG. 6A or Table 3 (in FIG. 8).

In certain embodiments, the one or more modulators comprises one or more inhibitors (of up-regulated genes) or one or more of activators (of down-regulated genes) of one or more of the genes listed in FIG. 6A or Table 3.

In certain embodiments, the methods for treating an apicomplexan parasite infection include administering to the subject an amount effective to treat the infection of one or more of inhibitors of up-regulated genes listed in FIG. 6A or Table 3. For example, in certain embodiments, the method comprises administering to the subject an amount effective to treat the infection of one or more of inhibitors of one or more gene listed in Table 3 as entry No. 213-937. In certain other embodiments, the method comprises administering to the subject an amount effective to treat the infection of one or more of inhibitors of one or more gene listed in FIG. 6A in Row Nos. 1-10, 12-15, 17, 19-21, 23, 26, 27, 29-31, 33-36, and 41-44. In certain other embodiments, the method comprises administering to the subject an amount effective to treat the infection of one or more of inhibitors of one or more gene listed in FIG. 6A in Row No. 1-10, 12-15, 17, and 19-21. In certain other embodiments, the method comprises administering to the subject an amount effective to treat the infection of one or more of inhibitors of one or more genes selected from the group consisting of eukaryotic initiation factor-2a kinase (eif2k) gene (IF2K-B), GCN-1 (Row 14, ID #TGME49_231480), MIF4 domain (Row 13, ID #TGME49_269180), hypothetical protein (Row 10, ID #TGME49_206550), and hypothetical protein (Row 3, ID #TGME49_268240).

In certain embodiments, the inhibitor comprises an inhibitor selected from the group consisting of an antibody selective for the expressed protein from the one or more genes, and an inhibitory nucleic acid selective for the one or more gene selected from the group consisting of aptamer, small interfering RNA, small internally segmented interfering RNA, short hairpin RNA, microRNA, and antisense oligonucleotides.

In certain embodiments of the methods of the disclosure, the one or more of inhibitors, or one or more of activators, comprises a compound as described herein. For example, in certain embodiments, the compound may be a compound of formula (I). In another embodiment, the compound may be any one of compounds listed in Table 1. In another embodiment, the compound may be any one of compounds as disclosed in International Patent Publication WO 2017/112678 as noted above. In another embodiment, the compound is an activator of Ribosomal protein RPS13 (Row 24, ID #TGME49_270380).

In certain embodiments, the methods for treating an apicomplexan parasite infection include administering to a subject in need thereof an amount effective to treat the infection (i) one or more of eukaryotic initiation factor-2a kinase (eif2k) inhibitors selected from the group consisting of anti-eif2k antibody, anti-eif2k aptamer, eif2k small interfering RNA, eif2k small internally segmented interfering RNA, eif2k short hairpin RNA, eif2k microRNA, and eif2k antisense oligonucleotides, and (ii) one or more compounds as described here, such as the compounds of formula (I) as described herein, or compounds listed in Table 1, or compounds as disclosed in International Patent Publication WO 2017/112678 as noted above.

For example, in certain embodiments, the compound in (ii) is JAG021. In certain embodiments, JAG021 is administered as a nanoparticle formulation according to the disclosure.

As noted above, the disclosure also provides a method for identifying test compounds for apicomplexan parasite therapy. Such methods include identifying one or more of test compounds that modulate activity of one or more of the genes listed in FIG. 6A or Table 3 (in FIG. 8).

In certain embodiments, the identifying one or more test compounds includes identifying those that reduce activity and/or expression (e.g., inhibitors for up-regulated genes) or those that increase expression (e.g., activators for down-regulated genes) of one or more of the genes listed in FIG. 6A or Table 3.

For example, in certain embodiments, the method comprises identifying one or more test compounds that reduce activity and/or expression of one or more gene comprises the gene listed in Table 3 as entry No. 213-937. In certain embodiments, the method comprises identifying one or more test compounds that reduce activity and/or expression of one or more gene listed in FIG. 6A in Row Nos. 1-10, 12-15, 17, 19-21, 23, 26, 27, 29-31, 33-36, and 41-44. In certain other embodiments, the method comprises identifying one or more test compounds that reduce activity and/or expression of one or more gene listed in FIG. 6A as Row No. 1-10, 12-15, 17, and 19-21. In certain other embodiments, the method comprises identifying one or more test compounds that reduce activity and/or expression of one or more gene selected from the group consisting of eukaryotic initiation factor-2a kinase (eif2k) gene (IF2K-B), GCN-1 (Row 14, ID #TGME49_231480), MIF4 domain (Row 13, ID #TGME49_269180), hypothetical protein (Row 10, ID #TGME49_206550), and hypothetical protein (Row 3, ID #TGME49_268240). In certain other embodiments, the method comprises identifying one or more test compounds that increase activity and/or expression of Ribosomal protein RPS13 (Row 24, ID #TGME49_270380). In certain embodiments, the method comprises identifying one or more test compounds using any combination of the activity disclosed herein.

As noted above, the disclosure also provides a method for diagnosing an apicomplexan parasite infection (such as a T. gondii infection). Such methods include:
(a) determining an expression level of one or more of the up-regulated and/or down-regulated genes listed in FIG. 6A or Table 3 (in FIG. 8) in a biological sample from a subject; and
(b) identifying a subject as having an apicomplexan parasite infection if subject has:
   (i) an expression level of 1, 2, 3, 4, 5, or more up-regulated genes increased relative to a threshold,
   (ii) an activity level of protein expressed from 1, 2, 3, 4, 5, or more up-regulated genes increased relative to a threshold,
   (iii) an expression level of 1, 2, 3, 4, 5, or more down-regulated genes decreased relative to a threshold, and/or
   (iv) an activity level of protein expressed from 1, 2, 3, 4, 5, or more down-regulated genes decreased relative to a threshold.

For example, in certain embodiment, the method includes determining the expression level of one or more gene listed in Table 3 as entry No. 213-937, and/or determining an activity level of the protein product of one or more gene listed in Table 3 as entry No. 213-937.

In certain other embodiment, the method includes determining the expression level of one or more gene listed in FIG. 6A in Row Nos. 1-10, 12-15, 17, 19-21, 23, 26, 27, 29-31, 33-36, and 41-44, and/or determining an activity level of the protein product of one or more gene listed in FIG. 6A in Row Nos. 1-10, 12-15, 17, 19-21, 23, 26, 27, 29-31, 33-36, and 41-44.

In certain other embodiment, the method includes determining the expression level of one or more gene listed in FIG. 6A in Row No. 1-10, 12-15, 17, and 19-21, and/or determining an activity level of the protein product of one or more gene listed in FIG. 6A in Row No. 1-10, 12-15, 17, and 19-21.

In certain other embodiment, the method includes determining the expression level of Ribosomal protein RPS13 (Row 24, ID #TGME49_270380), and/or determining an activity level of the protein product of Ribosomal protein RPS13 (Row 24, ID #TGME49_270380).

### Pharmaceutical Compositions

In another aspect, the present disclosure provides pharmaceutical compositions comprising one or more of compounds as described herein (e.g., listed in claim 1 and/or with respect to formula (I)) and an appropriate carrier, solvent, adjuvant, or diluent. The exact nature of the carrier, solvent, adjuvant, or diluent will depend upon the desired use for the composition, and may range from being suitable or acceptable for veterinary uses to being suitable or acceptable for human use. The composition may optionally include one or more secondary therapeutic agents. In certain embodiments, the composition may include one or more secondary apicomplexan parasite infection therapeutic agents.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders, such as steroids, methotrexate, etc. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries, which facilitate processing of the compounds into preparations, which can be used pharmaceutically.

The compounds may be formulated in the pharmaceutical composition per se, or in the form of a hydrate, solvate, N-oxide or pharmaceutically acceptable salt. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device, which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s), which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Definitions

The following terms and expressions used herein have the indicated meanings.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “—”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” unless a dash indicates otherwise. For example, alkyl, alkyl-, and -alkyl indicate the same functionality.

Further, certain terms herein may be used as both monovalent and divalent linking radicals as would be familiar to those skilled in the art, and by their presentation linking between two other moieties. For example, an alkyl group can be both a monovalent radical or divalent radical; in the latter case, it would be apparent to one skilled in the art that an additional hydrogen atom is removed from a monovalent alkyl radical to provide a suitable divalent moiety.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms, unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂, —CH₂CH₂, —CH₂CH₂CHC(CH₃)—, —CH₂CH(CH₂CH₃)CH₂—.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “hydroxy” as used herein, means an —OH group.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a parasite with a compound includes the administration of a compound described herein to an individual or patient, such as a human, infected with the parasite, as well as, for example, introducing a compound into a sample containing a cellular or purified preparation containing the parasite.

As used herein, the term “individual” or “patient,” or “subject” used interchangeably, refers to any animal, including mammals, preferably mice, rats, other rodents, rabbits, dogs, cats, swine, cattle, sheep, horses, or primates, and most preferably humans.

As used herein, the phrase “amount effective”, “therapeutically effective amount” or “effective to treat” refers to the amount of active compound or pharmaceutical agent that elicits the biological or medicinal response that is being sought in a tissue, system, animal, individual or human by a researcher, veterinarian, medical doctor or other clinician.

In certain embodiments, a therapeutically effective amount can be an amount suitable for

(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; or
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Ed., Wiley-Interscience, 2001; or Vogel, A Textbook of Practical Organic Chemistry, Including Qualitative Organic Analysis, Fourth Ed., New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/A, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formula (I) can be prepared according to general procedures (below), and/or analogous synthetic procedures. One of skill in the art can adapt the reaction sequences of Example 1 and general procedures to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### Examples

The preparation and methods of use of the compounds of the disclosure are illustrated further by the following examples, which are not to be construed as limiting the disclosure in scope or spirit to the specific methods and compounds described in them.

### Example 1. General Synthesis and Properties of Tetrahydroquinolone Compounds

The THQ compounds were synthesized as described below. 10 mM stock solutions were made with 100% Dimethyl Sulfoxide (DMSO) and working concentrations were made with IMDM-C(1×, [+] glutamine, [+] 25 mM HEPES, [−] Phenol red, 10% FBS)[Gibco, Denmark]). Final compounds had >95% purity determined by high performance liquid chromatography (HPLC), high resolution mass spectrometry and NMR spectrometry. Liquid chromatography-mass spectrometry (LC-MS) and NMR spectrometry were used to determine the integrity and purity of all intermediates. THQ compounds were synthesized as described in Schemes 1 and 2, which describe compounds MJM170 and JAG21 as exemplars.

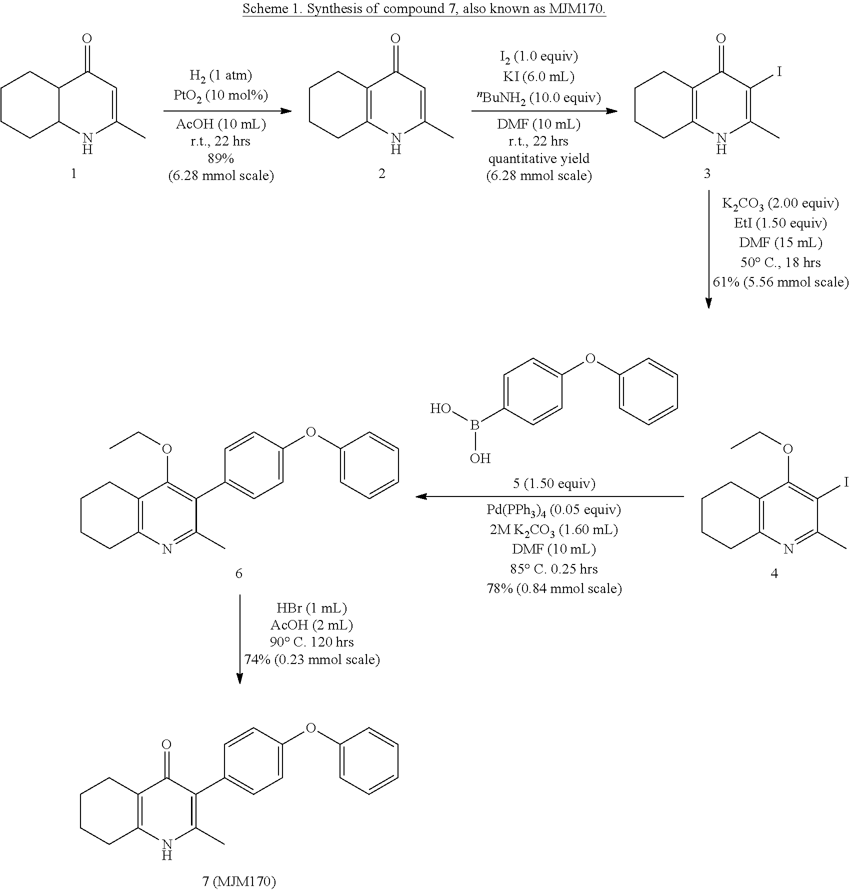

Scheme 2 provides two different routes to analogues of 7, route A or route B. Route A is a linear route to analogues requiring a Suzuki step to intermediate 12 from intermediates 4 and 10. Route B allows quicker access to analogues from intermediate 15 and derivatives can be synthesised via the Chan-Lam reaction to give final intermediate 12 by varying the boronic acid 16.

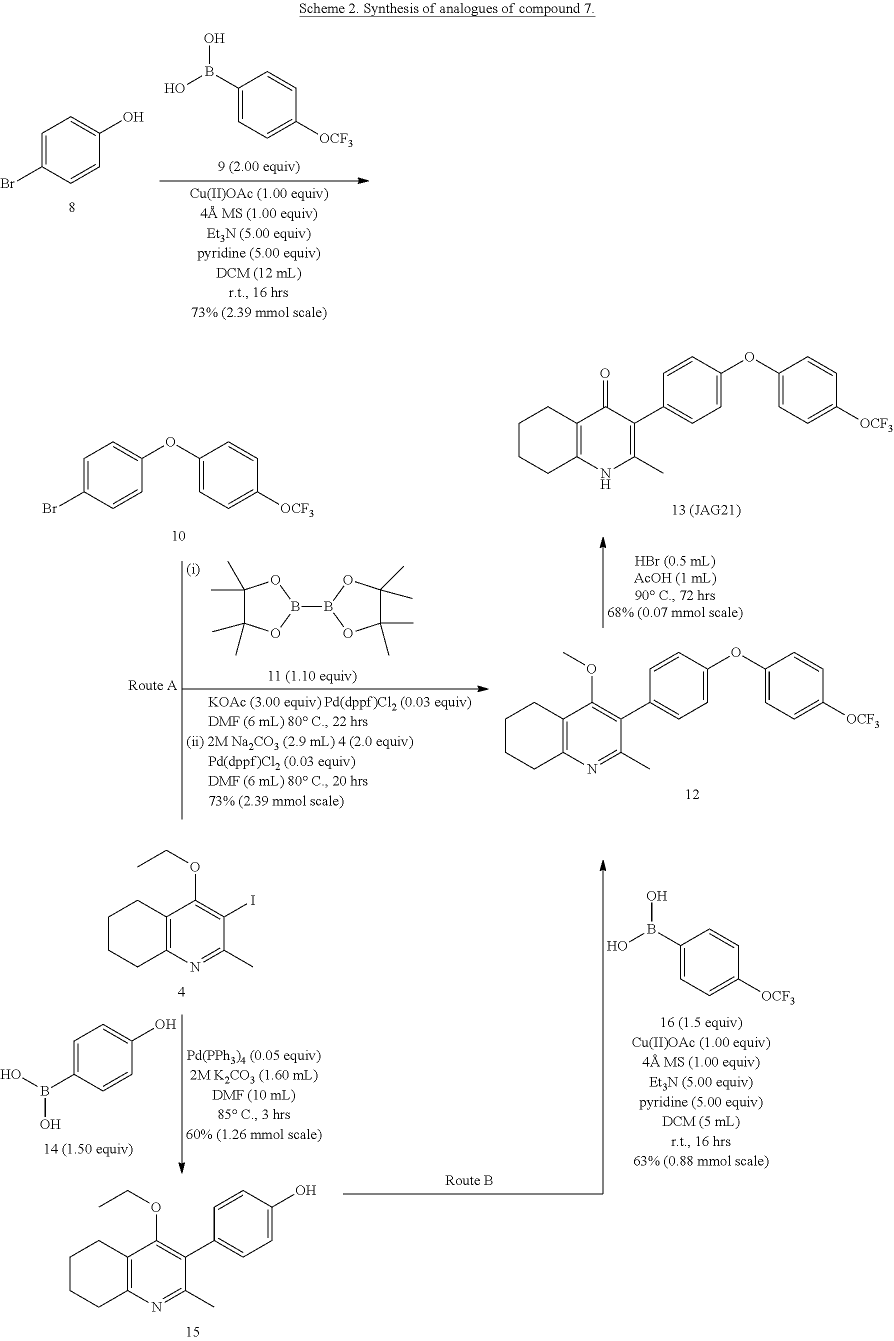

### Synthesis of 2-methyl-5,6,7,8-tetrahydroquinolin-4-one (2)

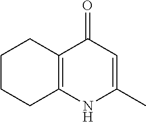

Platinum oxide (0.100 g, 10 mol %) was added to a solution of 4-hydroxy-2-methylquinoline (1, 1.00 g, 6.28 mmol, 1.00 eq) in glacial N acetic acid (10.0 mL). The heterogeneous mixture was catalytically H hydrogenated under a balloon of hydrogen. After 22 hrs, TLC (10% MeOH-DCM) confirmed complete reaction. The mixture was filtered through celite under vacuum, washing thoroughly with EtOAc (50 mL). The filtrate was concentrated and the resulting residue purified by column chromatography (10% MeOH-DCM) to give the desired product as a pale yellow oil (0.917 g, 5.65 mmol, 89%); R_{f} 0.14 (10% MeOH-DCM); δ_{H} (300 MHz, CDCl₃) 1.74-1.76 (4H, m, CH₂), 2.29 (3H, s, Me), 2.49-2.52 (2H, m, CH₂), 2.67-2.70 (2H, m, CH₂), 6.16 (1H, s, Ar—H); bc (125 MHz, CDCl₃) 19.0 (Me), 21.8 (CH₂), 22.1 (CH₂), 27.1 (CH₂), 112.5 (CH), 122.4 (Cq), 146.4 (Cq), 147.0 (Cq), 178.3 (Cq); Spectroscopic data consistent with literature values (JMC, 1993, 36, 1245-54).

### Synthesis of 2-methyl-3-iodo-5,6,7,8-tetrahydroquinolin-4-one (3)

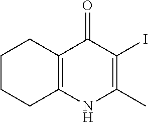

ⁿButylamine (6.20 mL, 62.8 mmol, 10.0 eq) was added to a suspension of 2-methyl-5,6,7,8-tetrahydroquinolin-4-one (2, 1.02 g, 6.28 mmol, 1.00 eq) in DMF (10.0 mL). To this heterogeneous mixture was added I₂ (1.60 g, 6.28 mmol, 1.00 eq) in a saturated solution of KI (6.00 mL). After 20 hrs stirring at R.T., a precipitate formed in the orange solution, excess iodine was quenched with 0.1 M sodium thiosulfate solution (10.0 mL). The precipitate was filtered by vacuum filtration, washed with distilled H₂O and dried (Na₂SO₄) to give the desired product as a colorless solid (1.76 g, 6.09 mmol, quantative yield); δ_{H} (300 MHz, DMSO-de) 1.61-1.70 (4H, m, CH₂), 2.29 (2H, t, J 6.0, CH₂), 2.43 (2H, s, CH₂), CH₃ under DMSO peak.

### Synthesis of 2-methyl-3-iodo-4-ethoxy-5,6,7,8-tetrahydroquinoline (4)

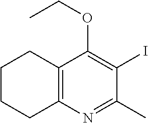

Potassium carbonate (1.53 g, 11.1 mmol, 2.00 eq) was added to a heterogeneous mixture of 2-methyl-3-iodo-5,6,7,8-tetrahydroquinolin-4-one (3, 1.60 g, 5.56 mmol, 1.00 eq) in DMF (15.0 mL), and the reaction heated to 50° C. for 30 minutes. The R.B. flask was removed from the heating mantle and ethyl iodide (0.67 mL, 8.33 mmol, 1.50 eq) was added dropwise. The reaction was then heated at 50° C. for 18 hrs. The reaction was cooled to R.T., quenched with water (40 mL). The resulting emulsion formed which was extracted with EtOAc (50 mL). EtOAc layer were washed with water (3×30 mL), brine (3×30 mL), dried (Na₂SO₄) and concentrated to give a pale yellow oil (1.09 g, 3.44 mmol, 61%); R_{f} 0.88 (1:1 Pet-EtOAc); HPLC (RT=1.67 minutes); LCMS (Method A), (RT=1.6 min, m/z (ES) Found MH⁺ 318.0); δ_{H} (500 MHz, CDCl₃) 1.49 (3H, t, J 7.0, ethoxy CH₃), 1.73-1.78 (2H, m, CH₂) 1.84-1.88 (2H, m, CH₂), 2.78-2.69 (5H, m, CH₂ & CH₃), 2.84 (2H, t, J 6.5, CH₂), 3.97 (2H, q, J 7.0, OCH₂); bc (125 MHz, CDCl₃) 15.6 (CH₃), 22.3 (CH₂), 22.8 (CH₂), 23.6 (CH₂), 29.3 (CH₃), 32.0 (CH₂), 68.4 (OCH₂), 90.9 (Cq), 124.5 (Cq), 158.3 (Cq), 158.9 (Cq), 163.9 (Cq).

### Synthesis of 2-methyl-3-(4-phenoxyphenyl)-4-ethoxy-5,6,7,8-tetrahydroquinoline (6)

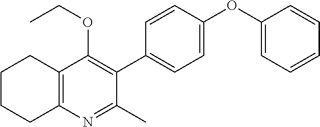 [SMILES: CCOc1c2c(nc(C)c1-c1ccc(Oc3ccccc3)cc1)CCCC2]

2-Methyl-3-iodo-4-ethoxy-5,6,7,8-tetrahydroquinoline (4, 0.266 g, 0.839 mmol, 1.00 eq), Pd(PPh₃)₄ (0.048 g, 0.0419 mmol, 5 mol %) and 4-phenoxyphenylboronic acid (5, 0.270 g, 1.26 mmol, 1.50 eq) were charged to a R.B. flask under N₂(g). Degassed DMF (10.0 mL) was added to the flask followed by 2M K₂CO₃ (1.60 mL). The flask was heated to 85° C. under N₂(g). After 15 minutes, TLC (4:1 Pet-EtOAc) confirmed reaction was complete. The reaction was cooled and diluted with EtOAc (15 mL), filtered through celite and partitioned between EtOAc (10 mL) and H₂O (25 mL). Combined organics were washed with H₂O (3×30 mL), then brine (3×30 mL), dried (Na₂SO₄) and concentrated to give a red oil which was purified by column chromatography (3:1 Pet-EtOAc), to give the desired product as a pale yellow oil (0.235 g, 0.655 mmol, 78%); R_{f} 0.31 (3:1 Pet-EtOAc); HPLC (RT=3.08 minutes); bH (300 MHz, CDCl₃) 1.04 (3H, t, J 7.0, ethoxy CH₃), 1.76-1.93 (4H, m, 2×CH₂), 2.32 (3H, s, CH₃) 2.72 (2H, t, J 6.0, CH₂), 2.91 (2H, t, J 6.5, CH₂), 3.50 (2H, q, J 7.0, OCH₂), 7.05-7.16 (5H, m, Ar—H), 7.20-7.29 (2H, m, Ar—H), 7.31-7.43 (2H, m, Ar—H); δ_{c} (125 MHz, CDCl₃) 15.7 (CH₃), 22.5 (CH₂), 23.0 (CH₃), 23.3 (CH₂), 23.4 (CH₂), 32.7 (CH₂), 68.2 (OCH₂), 118.6 (CH), 118.9 (CH), 123.4 (CH), 126.8 (Cq), 129.8 (CH), 131.5 (CH), 154.9 (Cq), 156.5 (Cq), 157.1 (Cq), 157.3 (Cq); m/z (ES) (Found: MH+, 360.1973. C₂₄H2eNO₂ requires MH, 360.1964).

### Synthesis of 2-methyl-3-(4-phenoxyphenyl)-4-ethoxy-5,6,7,8-tetrahydroquinoline (7, MJM170)

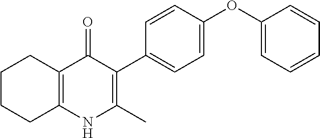 [SMILES: Cc1[nH]c2c(c(=O)c1-c1ccc(Oc3ccccc3)cc1)CCCC2]

Aqueous hydrobromic acid (>48%) (1.00 mL) was added to a solution of 2-methyl-3-(4-phenoxyphenyl)-4-ethoxy-5,6,7,8-tetrahydroquinoline (6, 0.226 g, 0.630 mmol, 1.00 eq) in glacial acetic acid (2 mL). The reaction was stirred at 90° C. for 5 days, monitoring by LMCS. The reaction was cooled to R.T. and the pH adjusted to pH 5 with 2M NaOH. The precipitate was collected by vacuum filtration and recrystallized from MeOH:H₂O to give the desired product as an off-white solid (0.155 g, 0.467 mmol, 74%); HPLC (RT=2.56 minutes); δ_{H} (500 MHz, DMSO-de) 1.66-1.72 (4H, m, 2×CH2), 2.08 (3H, s, CH₃) 2.31 (2H, t, J 6.0, CH₂), 2.56 (2H, t, J 6.0, CH₂), 6.99 (2H, d, J 8.5, Ar—H), 7.06 (2H, d, J 7.5, Ar—H), 7.14-7.18 (3H, m, Ar—H), 7.40-7.43 (2H, m, Ar—H), 11.0 (1H, s, NH); 6c (125 MHz, DMSO-de) 17.7 (CH₃), 21.5 (CH₂), 21.8 (CH₂), 21.9 (CH₂), 26.2 (CH₂), 117.8 (CH), 118.6 (CH), 121.2 (Cq), 123.3 (CH), 123.7 (Cq), 130.0 (CH), 131.4 (Cq), 132.3 (CH), 142.3 (Cq), 143.2 (Cq), 155.0 (Cq), 156.8 (Cq), 175.4 (Cq); m/z (ES) (Found: MH, 332.1654. C₂₂H22NO₂ requires MH, 332.1645).

### Synthesis of 1-(4-bromophenyl)-4-(trifluoromethoxy)benzene (10)

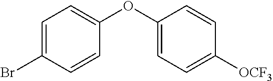 [SMILES: FC(F)(F)Oc1ccc(Oc2ccc(Br)cc2)cc1]

Copper (II) acetate (0.435 g, 2.39 mmol, 1.00 eq) was added to a suspension of 4-bromophenol (8, 0.414 g, 2.39 mmol, 1.00 eq), 4-trifluoromethoxybenzeneboronic acid (9, 0.983 g, 4.79 mmol, 2.00 eq) and 4 Å molecular sieves (0.566 g) in DCM (12 mL) at R.T. A solution of triethylamine (1.7 mL, 11.9 mmol, 5.00 eq) and pyridine (1 mL, 11.9 mmol, 5.00 eq) was added and the reaction was stirred for 16 hrs, open to the atmosphere. After 18 hrs, the reaction was quenched with 0.5 M HCl (20 mL) and the organic layer washed with water (20 mL), brine (20 mL), dried (Na₂SO₄) and concentrated to give a red oil which was purified by column chromatography (hexane) to give the desired product as a colorless oil (0.582 g, 1.75 mmol, 73%); Rᵣ 0.58 (hexane).

### Synthesis of 2-methyl-3-(4-hydroxyphenyl)-4-ethoxy-5,6,7,8-tetrahydroquinolin-4-one (15)

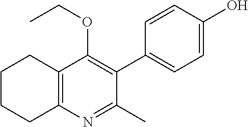 [SMILES: CCOc1c2c(nc(C)c1-c1ccc(O)cc1)CCCC2]

2-Methyl-3-iodo-4-ethoxy-5,6,7,8-tetrahydroquinoline (4, 0.400 g, 1.26 mmol, 1.00 eq), Pd(PPh₃)₄(0.073 g, 0.06 mmol, 5 mol %) and 4-hydroxylphenylboronic acid (14, 0.260 g, 1.89 mmol, 1.50 eq) were charged to a R.B. flask under N₂(g). Degassed DMF (10.0 mL) was added to the flask followed by 2M K₂CO₃ (3.00 mL). The flask was heated to 85° C. under N₂(g). After 3 hrs, TLC (EtOAc) confirmed reaction was complete. The reaction was cooled to 50° C., diluted with EtOAc (15 mL) and activated charcoal was added. After stirring for 30 minutes, the mixture was filtered through celite and partitioned between EtOAc (10 mL) and H₂O (25 mL). Combined organics were washed with H₂O (3×30 mL), then brine (3×30 mL), dried (Na₂SO₄) and concentrated to give a brown solid which was triturated with diethyl ether to give the desired product as a pale red crystalline solid (0.220 g, 0.777 mmol, 60%); R_{f} 0.22 (EtOAc); m.p. 225-226° C. (EtOAc); 6H (500 MHz, MeOD-d₄) 7.07 (d, J=8.6 Hz, 2H, H-3 & 5), 6.86 (d, J=8.6 Hz, 2H, H-2 & 6), 3.51 (q, J=7.0 Hz, 2H, CH₃CH₂O), 2.83 (t, J=6.3 Hz, 2H, H-8′), 2.72 (t, J=6.1 Hz, 2H, H-5′), 2.23 (s, 3H, Me), 1.95-1.72 (m, 4H, H-6′ & 7′), 1.00 (t, J=7.0 Hz, 3H, CH₃CH₂O); bc (125 MHz, MeOD-d₄) 164.0 (Cq), 158.1 (C-1), 157.4 (C_{q}), 156.1 (C_{q}), 132.2 (C-3 & 5), 129.1 (Cq), 127.9 (Cq), 124.9 (Cq), 116.2 (CH), 69.1 (OCH₂), 32.7 (CH₂), 23.9 (CH₂), 23.4 (CH₃), 22.9 (CH₂), 22.3 (CH₂), 15.7 (CH₃); m/z (ES) (Found MH+, 284.1664, C₁eH₂₁NO₂ requires MH, 284.1651).

### Synthesis of 2-methyl-3-(4-hydroxyphenyl)-4-ethoxy-5,6,7,8-tetrahydroquinolin-4-one (12)

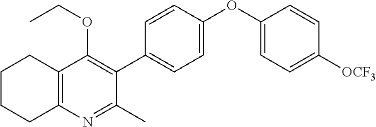 [SMILES: CCOc1c2c(nc(C)c1-c1ccc(Oc3ccc(OC(F)(F)F)cc3)cc1)CCCC2]

1-(4-bromophenyl)-4-(trifluoromethoxy) benzene (10, 0.100 g, 0.30 mmol, 1.00 eq), bis(pinacolato) diboron (1.10 eq), potassium acetate (3.00 eq) and Pd(dppf)Cl₂ (0.03 eq) were added to a oven-dried flask under inert (N₂) atmosphere. Anhydrous DMF (6 mL) was added and the reaction heated to 80° C. under N₂ (g). After 22 hrs, the reaction was cooled to R.T., fresh Pd(dppf)Cl₂ (0.03 eq) added, followed by 2-methyl-3-iodo-4-ethoxy-5,6,7,8-tetrahydroquinoline (4, 0.400 g, 1.26 mmol, 2.00 eq) and 2M Na₂CO₃ (2.9 mL). The reaction was heated to 80° C. for 20 hrs, cooled, diluted with EtOAc (20 mL), filtered through celite and partitioned between EtOAc (20 mL) and H₂O (20 mL). Combined organics were washed with brine (3×20 mL), dried (Na₂SO₄) and concentrated to give a brown solid which was purified by column chromatography (3:1 Pet-EtOAc) to give the desired product as a colorless oil (30 mg, 0.07 mmol, 23%); HPLC (RT=2.41 minutes); 6H (500 MHz, acetone) 7.28 (d, J=8.7 Hz, 2H, H-2′ & 6′), 7.26 (d, J=9.1 Hz, 2H, H-2″ & 6″), 7.09 (d, J=9.1 Hz, 2H, H-3″ & 5″), 7.07 (d, J=8.7, 2H, H-3′ & 5′), 3.52 (q, J=7.0 Hz, 2H, CH₃CH₂O), 2.85 (t, J=6.5 Hz, 2H, H-8), 2.78 (t, J=6.2 Hz, 2H, H-5), 2.26 (s, 3H, Me), 1.89-1.81 (m, 2H, H-7), 1.81-1.72 (m, 2H, H-6), 0.93 (t, J=7.0 Hz, 3H, CH₃CH₂O); bc (125 MHz, acetone) δ 161.9 (Cq), 157.1 (Cq), 156.5 (Cq), 156.0 (Cq), 154.5 (Cq), 145.3 (Cq), 132.5 (Cq), 132.0 (CH), 126.7 (Cq), 123.0 (CH), 119.8 (CH), 119.0 (CH), 68.0 (OCH₂), 32.5 (CH₂), 23.0 (CH₂), 22.9 (CH₃), 22.7 (CH₂), 22.5 (CH₂), 15.05 (CH₃); m/z (ES) (Found: MH*, 444.1792. C₂₅H₂₄F3NO₃ requires MH, 444.1781).

### Synthesis of 2-methyl-3-(4-(4-(trifluoromethoxy)phenoxy)phenyl)-5,6,7,8-tetrahydroquinolin-4-one (13, JAG21)

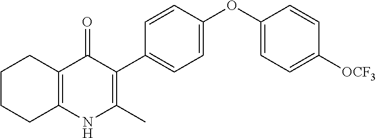 [SMILES: Cc1[nH]c2c(c(=O)c1-c1ccc(Oc3ccc(OC(F)(F)F)cc3)cc1)CCCC2]

Aqueous hydrobromic acid (>48%) (1.00 mL) was added to a solution of 2-methyl-3-(4-phenoxyphenyl)-4-ethoxy-5,6,7,8-tetrahydroquinoline (12, 30.0 mg, 0.07 mmol, 1.00 eq) in glacial acetic acid (2 mL). The reaction was stirred at 90° C. for 3 days, monitoring by LMCS. The reaction was cooled to R.T. and the pH adjusted to pH 5 with 2M NaOH. The precipitate was collected by vacuum filtration and recrystallized from MeOH:H₂O to give the desired product as a colorless solid (25.0 mg, 0.06 mmol, 68%); m.p.>250° C.; HPLC (RT=2.78 minutes); δ_{H} (500 MHz, DMSO-de) 11.07 (s, 1H, NH), 7.40 (d, J=8.5 Hz, 2H, H-2′ & 6′), 7.19 (d, J=8.6 Hz, 2H, H-3″ & 5″), 7.13 (d, J=9.0 Hz, 2H, H-3′ & 5′), 7.02 (d, J=8.5 Hz, 2H, H-2″ & 6″), 2.54 (t, J=6.0 Hz, 2H, H-8), 2.28 (t, J=5.9 Hz, 2H, H-5), 2.07 (s, 3H, Me), 1.71 (m, 2H, H-7), 1.65 (m, 2H, H-6); δ_{c} (125 MHz, DMSO-do) 175.7 (Cq), 155.9 (Cq), 154.5 (Cq), 143.5 (Cq), 143.2 (Cq), 142.2 (Cq), 132.5 (CH), 132.2 (Cq), 123.6 (Cq), 123.0 (CH), 121.3 (Cq), 119.6 (CH), 118.2 (CH), 26.2 (CH₂), 21.9 (CH₂) 21.8 (CH₂), 21.5 (CH₂), 17.7 (CH₃); m/z (ES) (Found: MH, 416.1492. C₂₃H₂₀F₃NO₃ requires MH, 416.1473).

Building blocks 1, 8, 9 and 14 were varied to create the complete series shown in Table 1, which provides characteristics and effects of compounds on inhibition of T.gondii replication and enzyme activity of the compounds of the disclosure (solubility in PBS 7.4, toxicity against HFF, predicted half-life, and inhibitory effect of compounds on RH strain tachyzoites and EGS strain bradyzoites in vitro and saffarine 0 assay enzyme activity). PBS Sol/Toxicity pH7.4 refers to solubility of the compound in Phosphate Buffered Saline (PBS) at pH7.4. Toxicity refers to the highest concentration tested that does not show toxicity to Human Foreskin Fibroblast (HFF) in tissue culture in WST assay; T_{1/2}(H) refers to the predicted half-life in human liver microsomes; T₁₂ (M) refers to the predicted half-life in mouse liver microsomes. Tachy/Brady IC₅₀ was determined in studies in which cultures of parasites in HFF were treated with varying concentrations of the compound and there was 50% inhibition of the replication (number) of parasites. Parasites were RH-YFP expressing tachyzoites (Tachy) and EGS (Brady) strains. Studies of effects of inhibitors on HFF or on T.gondii tachyzoites were performed with triplicate wells in at least two biological replicate experiments. Studies of effects on bradyzoites were performed at least twice in at least two biological replicate experiments. Compounds with much less inhibition of mammalian than T. gondii cytochrome bc in the saffarine enzyme assay (indicated by **) provide potential to further develop compounds, if unanticipated toxicity occurs from JAG21.

TABLE 1

| | | PBS | | | Tachy/ |
|---|---|---|---|---|---|
| Compound | | Sol/Toxicity* | T_{1/2} | T_{1/2} | Brady IC₅₀ |
| Code | Structure | pH7.4 μM | (H) | (M) | μM |
| JAG021 | 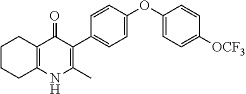 [SMILES: Cc1[nH]c2c(c(=O)c1-c1ccc(Oc3ccc(OC(F)(F)F)cc3)cc1)CCCC2] | 7.07/* | >7 days | 101.09 | 0.12/2 |
| JAG022 | 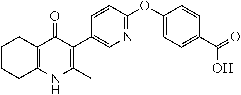 [SMILES: Cc1[nH]c2c(c(=O)c1-c1ccc(Oc3ccc(C(=O)O)cc3)nc1)CCCC2] | ND/5 | ND | ND | 7.6/ND |
| JAG046 | 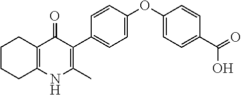 [SMILES: Cc1[nH]c2c(c(=O)c1-c1ccc(Oc3ccc(C(=O)O)cc3)cc1)CCCC2] | ND/5 | ND | ND | >10/ND |
| JAG047 | 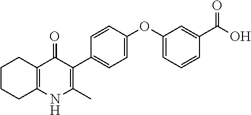 [SMILES: Cc1[nH]c2c(c(=O)c1-c1ccc(Oc3cccc(C(=O)O)c3)cc1)CCCC2] | ND/5 | ND | ND | >10/ND |
| JAG050 | 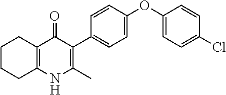 [SMILES: Cc1[nH]c2c(c(=O)c1-c1ccc(Oc3ccc(Cl)cc3)cc1)CCCC2] | 16.41/* | 99.04 | 68.55 | 0.085/2 |
| JAG062 | 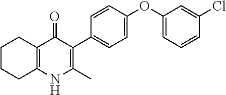 [SMILES: Cc1[nH]c2c(c(=O)c1-c1ccc(Oc3cccc(Cl)c3)cc1)CCCC2] | 0.33/* | 135.3 | 12.42 | 0.016/1 |
| JAG069 | 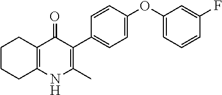 [SMILES: Cc1[nH]c2c(c(=O)c1-c1ccc(Oc3cccc(F)c3)cc1)CCCC2] | 0.5/* | 201.98 | 17.38 | 0.03/1 |
| JAG084 | 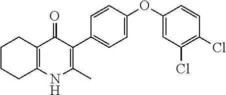 [SMILES: Cc1[nH]c2c(c(=O)c1-c1ccc(Oc3ccc(Cl)c(Cl)c3)cc1)CCCC2] | 0.68/* | ND | 63.1 | 0.055/1 |
| JAG204 | 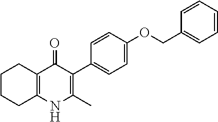 [SMILES: Cc1[nH]c2c(c(=O)c1-c1ccc(OCc3ccccc3)cc1)CCCC2] | ND/5 | ND | ND | 0.02/1 |
| JAG208 | 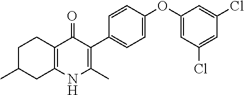 [SMILES: Cc1[nH]c2c(c(=O)c1-c1ccc(Oc3cc(Cl)cc(Cl)c3)cc1)CCC(C)C2] | ND/5 | ND | ND | 0.02/1 |
| JAG058 | 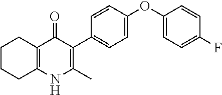 [SMILES: Cc1[nH]c2c(c(=O)c1-c1ccc(Oc3ccc(F)cc3)cc1)CCCC2] | 2.38/* | 263.1 | 39.17 | 0.04/1 |
| JAG063 | 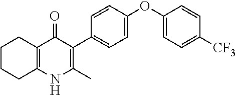 [SMILES: Cc1[nH]c2c(c(=O)c1-c1ccc(Oc3ccc(C(F)(F)F)cc3)cc1)CCCC2] | 0.45/* | 536.6 | 126.96 | 0.2/ND |
| JAG023 | 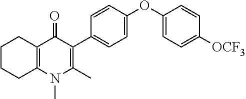 [SMILES: Cc1c(-c2ccc(Oc3ccc(OC(F)(F)F)cc3)cc2)c(=O)c2c(n1C)CCCC2] | ND/5 | ND | ND | 0.8/ND |
| JAG077 | 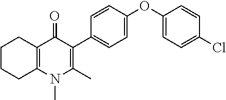 [SMILES: Cc1c(-c2ccc(Oc3ccc(Cl)cc3)cc2)c(=O)c2c(n1C)CCCC2] | ND/5 | ND | ND | 0.4/ND |
| AS006** | 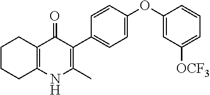 [SMILES: Cc1[nH]c2c(c(=O)c1-c1ccc(Oc3cccc(OC(F)(F)F)c3)cc1)CCCC2] | 0.55/* | ND | 25.88 | 0.06/1 |
| AS012** | 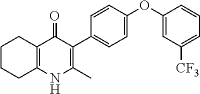 [SMILES: Cc1[nH]c2c(c(=O)c1-c1ccc(Oc3cccc(C(F)(F)F)c3)cc1)CCCC2] | 2.19/* | ND | 30.05 | 0.26/ND |
| AS021 | 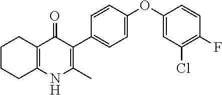 [SMILES: Cc1[nH]c2c(c(=O)c1-c1ccc(Oc3ccc(F)c(Cl)c3)cc1)CCCC2] | 2/* | ND | 41.09 | 0.065/1 |
| AS034 | 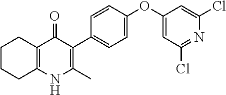 [SMILES: Cc1[nH]c2c(c(=O)c1-c1ccc(Oc3cc(Cl)nc(Cl)c3)cc1)CCCC2] | 5.05/* | ND | 24.93 | 0.28/ND |
| AS022 | 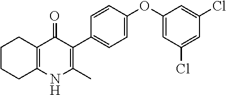 [SMILES: Cc1[nH]c2c(c(=O)c1-c1ccc(Oc3cc(Cl)cc(Cl)c3)cc1)CCCC2] | 6.25/* | ND | 28.62 | 0.03/1 |
| JAG091 | 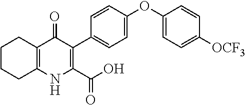 [SMILES: O=C(O)c1[nH]c2c(c(=O)c1-c1ccc(Oc3ccc(OC(F)(F)F)cc3)cc1)CCCC2] | ND/5 | ND | ND | >10/ND |
| JAG092 | 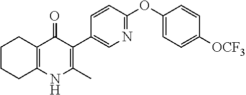 [SMILES: Cc1[nH]c2c(c(=O)c1-c1ccc(Oc3ccc(OC(F)(F)F)cc3)nc1)CCCC2] | ND/5 | ND | ND | 1/ND |
| JAG095 | 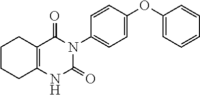 [SMILES: O=c1[nH]c2c(c(=O)n1-c1ccc(Oc3ccccc3)cc1)CCCC2] | ND/5 | ND | ND | >10/ND |
| JAG099 | 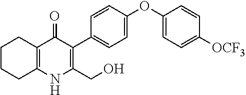 [SMILES: O=c1c2c([nH]c(CO)c1-c1ccc(Oc3ccc(OC(F)(F)F)cc3)cc1)CCCC2] | 4.05/* | ND | ND | 0.38/ND |
| AS032* | 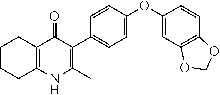 [SMILES: Cc1[nH]c2c(c(=O)c1-c1ccc(Oc3ccc4c(c3)OCO4)cc1)CCCC2] | ND/5 | ND | ND | 0.2/ND |
| AS033 | 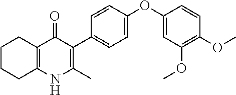 [SMILES: COc1ccc(Oc2ccc(-c3c(C)[nH]c4c(c3=O)CCCC4)cc2)cc1OC] | ND/5 | ND | ND | ND |
| JAG100** | 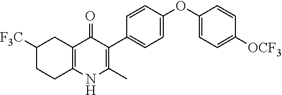 [SMILES: Cc1[nH]c2c(c(=O)c1-c1ccc(Oc3ccc(OC(F)(F)F)cc3)cc1)CC(C(F)(F)F)CC2] | ND/5 | ND | ND | >10/ND |
| JAG106 | 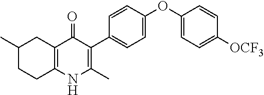 [SMILES: Cc1[nH]c2c(c(=O)c1-c1ccc(Oc3ccc(OC(F)(F)F)cc3)cc1)CC(C)CC2] | ND/5 | ND | ND | 2.5/ND |
| JAG107 | 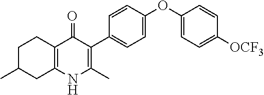 [SMILES: Cc1[nH]c2c(c(=O)c1-c1ccc(Oc3ccc(OC(F)(F)F)cc3)cc1)CCC(C)C2] | 0.03 | ND | 111.93 | 0.05/ND |
| JAG121 | 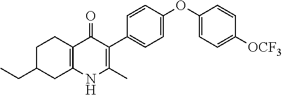 [SMILES: CCC1CCc2c([nH]c(C)c(-c3ccc(Oc4ccc(OC(F)(F)F)cc4)cc3)c2=O)C1] | 0.16 | ND | 63.28 | 0.055/ND |
| JAG162** | 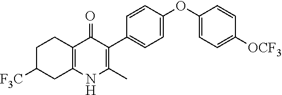 [SMILES: Cc1[nH]c2c(c(=O)c1-c1ccc(Oc3ccc(OC(F)(F)F)cc3)cc1)CCC(C(F)(F)F)C2] | 0.02 | ND | 144.43 | 0.3/ND |
| JAG094 | 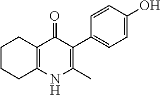 [SMILES: Cc1[nH]c2c(c(=O)c1-c1ccc(O)cc1)CCCC2] | ND/5 | ND | ND | 1/ND |
| JAG171 | 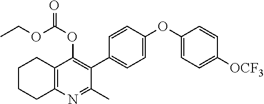 [SMILES: CCOC(=O)Oc1c2c(nc(C)c1-c1ccc(Oc3ccc(OC(F)(F)F)cc3)cc1)CCCC2] | ND/5 | ND | ND | 0.1/ND |
| JAG174 | 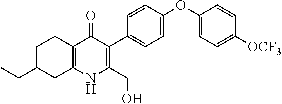 [SMILES: CCC1CCc2c([nH]c(CO)c(-c3ccc(Oc4ccc(OC(F)(F)F)cc4)cc3)c2=O)C1] | ND/5 | ND | ND | 0.38/ND |
| JAG187** | 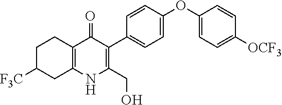 [SMILES: O=c1c2c([nH]c(CO)c1-c1ccc(Oc3ccc(OC(F)(F)F)cc3)cc1)CC(C(F)(F)F)CC2] | ND/5 | ND | ND | 2/ND |
| JAG193 | 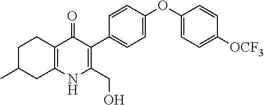 [SMILES: CC1CCc2c([nH]c(CO)c(-c3ccc(Oc4ccc(OC(F)(F)F)cc4)cc3)c2=O)C1] | ND/5 | ND | ND | 0.05/ND |
| NP032 | 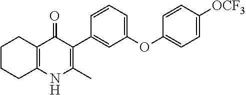 [SMILES: Cc1[nH]c2c(c(=O)c1-c1cccc(Oc3ccc(OC(F)(F)F)cc3)c1)CCCC2] | ND/5 | ND | ND | 0.2/ND |
| NP034 | 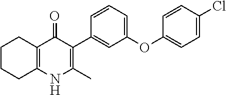 [SMILES: Cc1[nH]c2c(c(=O)c1-c1cccc(Oc3ccc(Cl)cc3)c1)CCCC2] | ND/5 | ND | ND | 0.08/ND |
| NP035 | 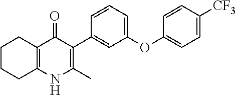 [SMILES: Cc1[nH]c2c(c(=O)c1-c1cccc(Oc3ccc(C(F)(F)F)cc3)c1)CCCC2] | ND/5 | ND | ND | 0.65/ND |
| JAG199 | 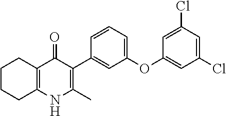 [SMILES: Cc1[nH]c2c(c(=O)c1-c1cccc(Oc3cc(Cl)cc(Cl)c3)c1)CCCC2] | ND/5 | ND | ND | 0.2/ND |
| JAG200 | 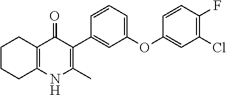 [SMILES: Cc1[nH]c2c(c(=O)c1-c1cccc(Oc3ccc(F)c(Cl)c3)c1)CCCC2] | ND/5 | ND | ND | 0.06/ND |
| MJM170 | 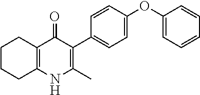 [SMILES: Cc1[nH]c2c(c(=O)c1-c1ccc(Oc3ccccc3)cc1)CCCC2] | 1.97/* | 146.33 | 20.97 | 0.03/4 |
| ELQ271 | 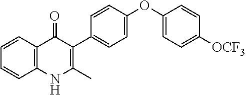 [SMILES: Cc1[nH]c2ccccc2c(=O)c1-c1ccc(Oc2ccc(OC(F)(F)F)cc2)cc1] | 0.15/* | 171.93 | 448.13 | 0.03/5 |
| JAG039 | 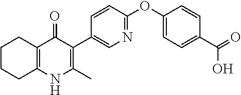 [SMILES: Cc1[nH]c2c(c(=O)c1-c1ccc(Oc3ccc(C(=O)O)cc3)nc1)CCCC2] | ND/5 | ND | ND | 7.6/ND |
| JAG129 | 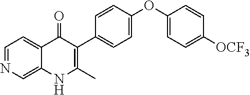 [SMILES: Cc1[nH]c2cnccc2c(=O)c1-c1ccc(Oc2ccc(OC(F)(F)F)cc2)cc1] | 5.12 | N.D. | | 0.085/ND |
| JAG006 | 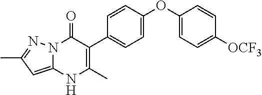 [SMILES: Cc1cc2[nH]c(C)c(-c3ccc(Oc4ccc(OC(F)(F)F)cc4)cc3)c(=O)n2n1] | ND/5 | ND | ND | 5/>10 |
| JAG013 | 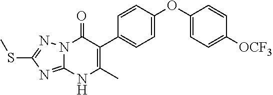 [SMILES: CSc1nc2[nH]c(C)c(-c3ccc(Oc4ccc(OC(F)(F)F)cc4)cc3)c(=O)n2n1] | ND/5 | ND | ND | 10/ND |
| JAG014 | 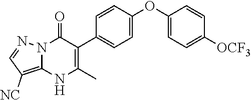 [SMILES: Cc1[nH]c2c(C#N)cnn2c(=O)c1-c1ccc(Oc2ccc(OC(F)(F)F)cc2)cc1] | ND/5 | ND | ND | >10/ ND |
| JAG015 | 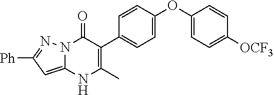 [SMILES: Cc1[nH]c2cc(-c3ccccc3)nn2c(=O)c1-c1ccc(Oc2ccc(OC(F)(F)F)cc2)cc1] | ND/5 | ND | ND | 10/ND |
| MJM129 | 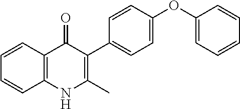 [SMILES: Cc1[nH]c2ccccc2c(=O)c1-c1ccc(Oc2ccccc2)cc1] | ND/5 | ND | ND | 0.05/>10 |
| MJM136 | 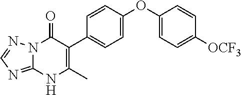 [SMILES: Cc1[nH]c2ncnn2c(=O)c1-c1ccc(Oc2ccc(OC(F)(F)F)cc2)cc1] | ND/5 | ND | ND | 3.1/>10 |
| MJM141 | 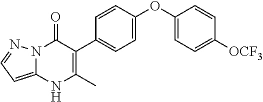 [SMILES: Cc1[nH]c2ccnn2c(=O)c1-c1ccc(Oc2ccc(OC(F)(F)F)cc2)cc1] | 0.94/* | 278.33 | ND | 8.2/10 |

### Example 2. General In Vitro and In Vivo Methods

Toxoplasma gondii:

Parasite Strains (Isolates). RH-YFP tachyzoites, (McPhillie et al, 2016; Fomovska et al, 2012; Gubbels et al, 2003), EGS strain (Vidigal et al 2015; Paredes-Santos et al 2012; McPhillie et al, 2016), Pru-luciferase, Me49, and RPS13Δ on the RH strain background (Hutson et al, 2010) were prepared and passaged in human foreskin fibroblasts [HFF] as described.

In Vitro Challenge Assay for T.gondii

RH strain YFP Tachyzoites. Protocol was adapted from Fomovska et al. (2012; 2012a) for HFF. HFF were cultured on a flat, clear-bottomed, black 96-well plate to 90%-100% confluence. IMDM (1×, [+] glutamine, [+] 25 mM HEPES, [+] Phenol red, 10% FBS [Gibco, Denmark]) was removed and replaced with IMDM-C(1×, [+] glutamine, [+] 25 mM HEPES, [−] Phenol red, 10% FBS)[Gibco, Denmark]). RH-YFP, lysed from host cells by passing twice through a 27-gauge needle, were counted, then diluted to 32,000/mL in IMDM-C. HFF were infected with 3200 RH-YFP, then returned to 37° C., CO₂ (5%) incubator for 1-2 hours for infection. Various concentrations of the compounds in 20 μL IMDM-C were added to each well. There were triplicates for each condition. Controls were pyrimethamine/sulfadiazine (standard treatment), 0.1% DMSO only, HFF only, and untreated cultures of HFF infected with 2 fold dilutions of YFP expressing parasites (called “YFP gradient” to establish amount of color from known numbers of YFP expressing parasites). Cells were incubated at 37° C. for 72 hours. Plates were read using a fluorimeter (Synergy H4 Hybrid Reader, BioTek) to ascertain amount of relative fluorescence units (RFU) YFP, to measure parasite burden after treatment. Data were collected using Gen5 software with IC₅₀ calculated by graphical analysis in Excel.

An initial screening assay of 10 μM, 1 μM, 100 nM, and 10 nM of the compounds was performed. Compounds were not considered effective or pursued for further analysis if there was no inhibition of tachyzoites at 1 μM. If compounds were effective at 1 μM, another experiment was performed to assess effect at 1 μM, 500 nM, 250 nM, 125 nM, 62.5 nM, and 31.25 nM.

Cytotoxicity Assays in parallel with RH Strain T.gondii in vitro studies. Toxicity assays used WST-1 cell proliferation reagent (Roche) as in Fomovska et al. (2012). HFF were grown on a flat, clear-bottomed, black 96-well plate. Confluent HFF were treated with inhibitory compounds at concentrations of 10 μM and 50 μM. Compounds were diluted in IMDM-C, and 20 μL were added to each designated well, with triplicates for each condition. A gradient with 2 fold-decreasing concentrations of DMSO from 10% to 0% in colorless, translucent IMDM-C was used as a control. The plate was incubated for 72 hours at 37° C. 10 μL WST-1 reagent (Roche) were added to each well. Cells were incubated for 30-60 minutes. Absorbance was read using a fluorimeter at 420 nm. A higher degree of color change (and absorbance) indicated mitochondrial activity and cell viability.

In vitro Challenge Assay for EGS strain Bradyzoites. HFF cells were grown in IMDM on removable, sterile glass cover slips in the bottom of a clear, flat-bottomed 24-well plate. Cultures were infected with 3×10⁴ EGS strain parasites per well, in 0.5 mL media. The plate was returned to incubator at 37° C. overnight. The following day, the media was removed. Colorless IMDM and compounds were added to make various concentrations of the drug. Total volume was 0.5 mL 2 wells had media only, as a control. Plates were returned to the 37° C. incubator for 72 hours, checked once each 24 hours. If tachyzoites were visible in the control before 72 hours, cells were fixed and stained.

Cells were fixed using 4% paraformaldehyde and stained with Fluorescein-labeled Dolichos Biflorus Agglutinin, DAPI, and BAG1. Disks were removed, mounted on glass slides, and visualized using microscopy (Nikon T17). Slides were scanned using a CRi Panoramic Scan Whole Slide Scanner and viewed using Panoramic Viewer Software. Effects of compounds were quantitated by counting cysts in controls and treated cultures. Dolichos staining delimited structures and single organisms that remained were counted in a representative field of view. This was then multiplied by a factor determined by the total area of the cover slip in order to estimate the number of cysts and organisms in each condition. When the following forms were observed: “true cysts” with a dolichos-staining wall, “pseudocysts” or tight clusters of parasites, and small organisms, if there were fewer than four parasites visible in a cluster, organisms were counted individually (as “small organisms”). The entire scanned coverslip with all fields was also reviewed by 3 observers to confirm consistency.

Synergy studies with RH strain YFP Tachyzoites. Atovaquone and pyrimethamine were used to test whether they are synergistic with JAG21. Serial dilutions of the combination of JAG21 and either atovaquone or pyrimethamine were used in an in vitro challenge assay as described above. The EC50 of each compound and the combination of two compounds were determined. The effect of the combination of drugs was calculated with the following formula: C=[A]c/[A]a+[B]c/[B]a. If C is lower than 1, the two compounds tested have synergistic effect; if C is greater than 1, the two compounds tested have antagonist effect and if C is 1 they are additive.

T.gondii and HFF Mitochondrial Membrane Potential Measurements—the mitochondrial membrane potential was measured by the safranine method according to Vercesi et al, 1998). Freshly egressed T. gondii tachyzoites were filtered and washed twice with intracellular buffer (125 mM sucrose, 65 mM KCl, 10 mM HEPES-KOH buffer, pH 7.2, 1 mM MgCl₂ and 2.5 mM potassium phosphate). After washing, the parasites were resuspended in the same buffer at 10⁹/ml. An aliquot of 50 μL of this suspension was added to a cuvette containing Safranin 0, 2.5 μM and Succinate 1 mM in final volume of 2 mL of the intracellular buffer. The fluorescence was measured with a Hitachi 7000 spectrofluorometer with setting Ex. 495/Em. 586. Once the baseline fluorescence is testable, 30 μM digitonin was added to permeabilize the parasites. 85 seconds after permeabilization, the THQ derivatives, dissolved in DMSO, were added. 5 μM of FCCP (Carbonyl cyanide-4-(trifluoromethoxy) phenylhydrazone) was used as an uncoupler reference for calculations and its effect was considered 100%. Similar conditions for measuring the mitochondrial potential of mammalian cells were used with the following changes: the mammalian cells were re-suspended at 10⁸/ml. 50 μl of mammalian cells was also used for each run in a cuvette with 2 μl solutions. The substrate used for mammalian cells was 5 mM glutamate and 5 mM malate. A higher concentration of digitonin (50 μM) was used to permeabilize the mammalian cells. The compounds were added at about 400 seconds after permeabilization. Each experiment was repeated at least three times in duplicates. Statistical analysis, unpaired student t test, was performed using GraphPad Prism 8.0 (GraphPad Software, Inc., San Diego, Calif.).

Structure Activity Relationship (SAR) and comparison of effect on T.gondii and HFF enzyme activity. The effects of changing R¹ as 7-Et, 7-Me, 6-CF₃, or 6-Me on activity against RH strain tachyzoites, kinetic solubility, and metabolic stability were compared. Kinetic solubility and metabolic stability in human or murine liver microsomes were measured. The hERG (human Ether-à-go-go-Related) liability was also determined. The hERG gene (KCNH2) encodes a protein Kᵥ11.1, the alpha subunit of a potassium ion channel. This channel conducts the rapid component of the delayed rectifier potassium current, IKr, which is critical for repolarization of cardiac action potentials. A reduction in hERG currents from adverse drug effects can lead to long QT interval syndromes. These syndromes are characterized by action potential prolongation, lengthening of the QT interval on surface EKG, and an increased risk for “torsade de pointes” arrhythmias and sudden death. The MDCK-MDR1 Permeability Assay was also performed. MDCK-MDR1 refers to the ability of a compound to permeate across membranes of MDCK-MDR1 (Madin Darby canine kidney [MDCK] cells with the MDR1 gene [ABCB1], the gene encoding for the efflux protein, P-glycoprotein (P-gp)) in vitro. Assessing transport in both directions (apical to basolateral and basolateral to apical across the cell monolayers enables an efflux ratio to be determined. This provides an indication as to whether a compound undergoes active efflux (mediated by P-gp). This provides a prediction of blood brain barrier (BBB) penetration potential/permeability and efflux ratio. Effect in CACO-2 (Colon Adenocarcinoma cells) as a permeability assay and on cytochrome P450 CYP 450 were also determined. CYP enzymes catalyze oxidative biotransformation (phase 1 metabolism) of most drugs—CYP enzymes, bind to membranes in a cell (cyto) and contain a heme pigment (chrome and P) that absorbs light at a wavelength of 450 nm when exposed to carbon monoxide. Metabolism of a drug by CYP enzymes is a major source of variability in drug effect. These were measured by Chem Partners. The relative effect on HFF and parasite enzymes also were compared.

RPS13Δ Tachyzoites in Human Primary Brain Neuronal Stem Cells in vitro for transcriptomics and transcriptomics analyses. Culture of Human Primary Brain Neuronal Stem Cells (NSC) was as described (McPhillie et al, 2016; Ngo et al, 2017); T. gondii RPS13Δ on RH strain background (Hutson et al, 2010) was used to infect the NSC as described (McPhillie et al, 2016; Ngo et al, 2017). RNA was isolated and prepared and used for transcriptomic experiments as described (McPhillie et al, 2016; Ngo et al, 2017). Briefly, NSC, initially isolated from a temporal lobe biopsy (Walton et a, 2006) were infected with either wild-type or RPS13Δ RH tachyzoites using biological duplicates at a multiplicity of infection of 2:1 and incubated as previously described (Ngo et al, 2017). Eighteen hours post-infection, extracellular parasites were washed out with cold PBS before total RNA extraction. Further isolation of the mRNA fraction was carried out with miRNeasy Mini Kit columns (Qiagen) following manufacturer instructions and Illumina barcoded mRNA sequencing libraries were constructed with TruSeq RNA Sample Preparation Kits v2 (Illumina). Libraries were sequenced as 100 bp single reads with Illumina HiSeq 2000 apparatus at a sequencing depth of about 3 Gbp per sample. Sequencing reads were mapped to the human (release GRCh38) and T. gondii ME49 strain (ToxoDB release 13.0) reference genome assemblies with hisat2 (Kim et al, 2015) and raw read counts were per gene were estimated with HTSeq (Anders et al, 2015). Identification of parasite genes that were differentially expressed between wild-type and RPS13Δ parasites was performed with the R package DESeq2 (Love et al, 2014) using a generalized linear model likelihood ratio test. Identification of orthologous genes between T. gondii and P. cynomolgi was carried out by best-reciprocal matches between T. gondii and P. cynomolgi proteomes using Blastp and a e-value cutoff of 1×10⁴. The list of Genes that are differentially expressed between P. cynomolgi hypnozoites and the liver-schizont stage was extracted from a previously published study by Cubi R. et al (Cubi et al, 20171). Gene set enrichment analysis was carried out with the GSEA tool (Subramanian et al, 2005) using T. gondii Gene Ontology and cell cycle gene sets developed by Croken M et al (Croken et al, 20141) and visualized with the Enrichment Map application in Cytoscape (Su et al, 2014).

### Type II Parasites In Vivo

IVIS. Balb/C mice were infected intraperitoneally (IP) with 20×10³ T. gondii (Prugneaud strain expressing luciferase) tachyzoites. Treatment began 2 hours later with JAG21 (5 mg/kg) which was dissolved in DMSO, administered IP in a total volume of 0.05 ml. Mice were imaged every second day starting on day 4 post infection using an IVIS Spectrum (Caliper Life Sciences) for minute exposures, with medium binning, 20 minutes post injection with 150 mg/kg of D-luciferin potassium salt solution.

Brain cysts. Brain cysts were searched for in paraffin imbedded tissue of the surviving Prugneaud strain infected treated Balb/C mice in the IVIS study, 30 days after infection which was 16 days after treatment had been discontinued. All treated mice had survived. There were no surviving untreated mice in those experiments.

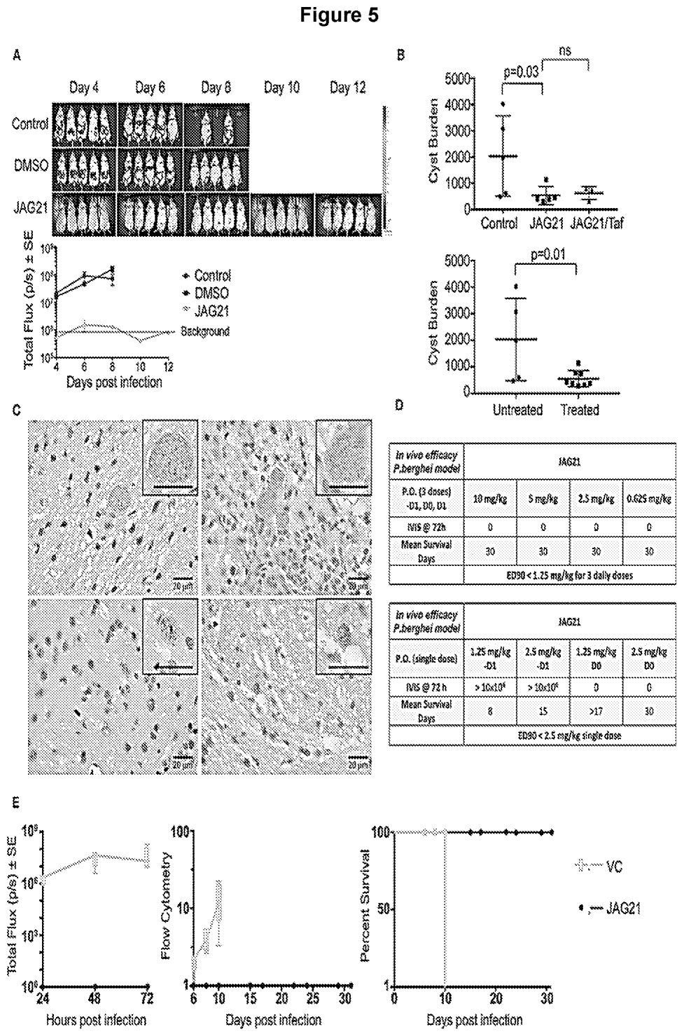

In separate experiments, Balb/C mice were infected IP with 20×10³ T. gondii Me49 strain tachyzoites. In these separate studies of mice with established chronic infection, after 30 days, IP treatment with JAG21 was begun each day for 14 days. JAG21 was dissolved in DMSO and administered IP in a total volume of 0.05 mL. In experiments when tafenoquine was administered alone or with JAG21 in some groups 3 mg/kg tafenoquine was administered once on day −1 from when JAG21 treatment was initiated. Cysts in brain were quantitated on day 30, 16 days after discontinuing JAG21. Immunoperoxidase staining was performed. Parasite burden was quantitated in two ways. The first was using a positive pixel count algorithm of Aperio ImageScope software. Positive pixels were normalized to tissue area (mm²). Briefly, automated quantitation was done by counting positive pixels per square area. The entire brain in one section was scanned for each mouse. The Cyst burden was quantitated as units of positive pixels per mm2. The average±S.E.M. numbers of mm2 per slide quantitated was 30.2±1.6 square mm per mouse for this quantification. Each highpower field of view shown in FIG. 5C is approximately 0.02 mm² per field of view. Cysts on each slide for each condition in two biological replicate experiments were also quantitated by 2 separate observers independently and results compared with automated counting, separately.

RPS13 Δ In vivo. This G1 arrested parasite persists in tissue culture for prolonged times in the absence of tetracycline (Hutson et al 2010), but in immune competent mice it cannot be rescued with teteracycline, or LNAME (L-N^{G}-Nitro arginine methyl ester, an analog of arginine) used as an antagonist of nitric oxide synthase (NOS) that inhibits NO production, or both together (Hutson et al. 2010).

In pilot studies, herein, interferon α receptor knockout mice that were not treated were observed following infection. At 7 and at 14 days following infection, spleen and liver were removed and immune peroxidase stained. At 14 days a group of mice were treated with anhydrotetracycline and when a subset of these mice died their spleen and liver were removed and immune peroxidase stained.

As in the pilot studies, this RPS13 Δ parasite also was used to infect interferon α receptor knockout mice in a treatment study. The design of this experiment with these immune compromised mice is shown in FIG. 6. In this separate study, groups of mice were infected with RPS13 Δ. They were treated with tafenoquine on day −1, or JAG21 for 14 days 2 hours after infection, or the two together with tafenoquine on day −1 and JAG21 for the first 14 days, or with diluent only for 14 days, as described above. For the initial 14 days no tetracycline was administered. After that time tetracycline was administered. Mice were observed each day. At the time they appeared to have substantial illness or at the termination of the experiment they were euthanized, tissues fixed in formalin and stained with hematoxylin and eosin or immunoperoxidase stained and parasite burden was assessed.

RH Challenge in a Study of Oral Administration of a Novel Nanoformulation of JAG21:

Nanoformulation of JAG21 for oral administration in T.gondii studies. JAG21 was prepared using hydroxyethyl cellulose (HEC) and Tween 80. Briefly, this dispersant solution containing 5 mg/mL HEC and 2 mg/mL Tween 80 in water was prepared. Solid JAG21 was added to 20 mg/mL, and the dispersion was sonicated for 60 seconds using a Sonics vc50 probe-tip sonicator set to 20 kHz to homogenize. Sonication was performed at room temperature. Aliquots of the homogeneous dispersion were frozen and lyophilized using a VirTis AdVantage freeze drier. These aliquots were stored at room temperature for 5-6 months. Prior to dosing, aliquots were reconstituted using water. Controls containing no JAG21 were also prepared. Following reconstitution with water, the dispersion was imaged using a Nikon ECLIPSE E200 optical microscope set to 40× magnification. The average particle size of the JAG21 dispersion in HEC/Tween 80 was determined using an in-house image analysis program This novel method to stably formulate JAG21 was discovered after all other studies were completed and this was the last experiment in this manuscript performed as a consequence.

RH YFP challenge. For studies of the nano formulated JAG 21, this was administered for one or three days by gavage in the doses shown in the results section. These C57BL6 background mice received 2000 RH tachyzoites IP on day the first day of the experiment and peritoneal fluid was collected 5 days later to quantitate filuorescence and numbers of parasites.

### Malaria:

Enzyme assays. P. falciparum 3D7 strain were obtained from the Liverpool School of Tropical Medicine. Protease cocktail inhibitor was obtained from Roche. Bradford protein assay dye reagent was obtained from Bio-Rad. All other reagents were obtained from Sigma-Aldrich. Decylubiquinol was produced as per Fisher et al. (2009). In brief, 25 mg of decylubiquinone were dissolved in 400 μl of nitrogen-saturated hexane. An equal volume of aqueous 1 M sodium dithionite was added, and the mixture vortexed until colorless. The organic phase containing the decylubiquinol was collected, the solvent was evaporated under N₂ and the decylubiquinol finally dissolved in 100 μl of 96% ethanol (acidified with 10 mM HCl). Concentrations of decylubiquinol was determined spectrophotometrically on a Cary 300 Bio UV/visible spectrophotometer (Varian, UK) from absolute spectra, using ε₂₈₈₋₃₂₀=8.1 mM-1·cm⁻¹. Decylubiquinol was stored at −80° C. and used within two weeks.

P. falciparum culture and extract preparation. P. falciparum strain 3D7 blood-stage cultures were contained a 2% suspension of O+ human erythrocytes in RPMI 1640 medium containing L-glutamine and sodium carbonate, and supplemented with 10% pooled human AB+ serum, 25 mM HEPES (pH 7.4) and 20 μM gentamicin sulphate. Cultures were grown under a gaseous headspace of 4% O₂ and 3% CO₂ in N₂ at 37° C. Cultures were grown to a parasitemia of 5% before use.

The protocol for the preparation of parasite extract was adapted from Fisher et al. Free parasites were prepared from infected erythrocytes pooled from five T75 flasks, by adding 5 volumes of 0.15% (w/v) saponin in phosphate-buffered saline (137 mM NaCl, 2.7 mM KCl, 1.76 mM K₂HPO₄, 8.0 mM Na₂HPO₄, 5.5 mM D-glucose, pH 7.4) for 5 min, followed by three washes by centrifugation in RPMI containing HEPES (25 mM), and a final resuspension in potassium phosphate buffer (50 mM K₂HPO₄, 50 mM KH₂PO₄, 2 mM EDTA, pH7.4) containing a protease inhibitor cocktail (Complete Mini; Roche). Parasite extract was then prepared by disruption with a sonicating probe for 5 s, followed by a 1 min rest period on ice to prevent the sample overheating. This process was performed three times. The parasite extract was used immediately. The protein concentration of the parasite extract was determined by Bradford protein assay (Bio-Rad).

Pfbc₁ native assay. P. falciparum bc₁ complex cytochrome c reductase (Pfbc₁) activity was measured by monitoring cytochrome c reduction at 550 versus 542 nm using a Cary 300 Bio UV-Visible Spectrophotometer (Varian, UK), using a protocol adapted from Fisher et al. (2009). The assay was performed in potassium phosphate buffer in a quartz cuvette and in a final volume of 700 μL. Potassium cyanide (10 μM), oxidized cytochrome c (30 μM), parasite extract (100 μg protein) and compound/DMSO were added sequentially to the cuvette, with mixing between each addition. Test compounds were added to a final concentration of 1 μM. DMSO (0.1% v/v) and atovaquone (1 μM), a known malarial cytochrome bc₁ complex inhibitor, were used as negative and positive controls respectively. The reaction was initiated by the addition of 50 μM decylubiquinol and allowed to proceed for 3 min.

Malaria parasite In vitro studies. Malaria potency testing in vitro was performed using 4 different P. falciparum strains, D6, TM91-C235, W2, and C2B. The D6 strain is a drug sensitive strain from Sierra Leone, the TM91-C235 strain is a multi-drug resistant strain from Thailand, the W2 strain is a chloroquine resistant strain from Thailand, and the C2B strain is a multi-drug resistant strain with resistance against atovaquone. These assays were performed as described below.

Compound Activity against Plasmodlum falciparum. Compound activity against P. falciparum, was tested using the Malaria SYBR Green I-Based Fluorescence (MSF) Assay. The complete method for performing this microtiter assay is described in previous work published by Plouffe et al. (2008) and Johnson et al (2007). In brief, this assay uses the binding of the fluorescent dye SYBR Green I to malaria DNA to measure parasite growth in the presence of two-fold diluted experimental or control. The relative fluorescence of the intercalated SYBR Green I proportional to parasite growth, and inhibitory compounds will result in lower observed fluorescence compared to untreated parasites.

Cytotoxicity assays in parallel with P.falclpaum assays In vitro. Toxicity studies also were performed with HepG2 cells (human liver cancer immortal cell line derived from the liver tissue of a 15-year-old African American, ATCC® HB-8065™) in parallel with the studies of P.falciparum, with inhibitors in vitro, as described in McPhillie et al (2016).

P. berghei causal prophylaxis In vivo model. P. berghei sporozoites were obtained from laboratory-reared female Anopheles stephensi mosquitoes which were maintained at 18 degrees C. for 17-22 days after feeding on a luciferase expressing P. berghei infected Swiss CD11CR Using a dissecting microscope, the salivary glands were extracted from malaria-infected mosquitoes and sporozoites were obtained. Briefly, mosquitoes were separated into head/thorax and abdomen. Thoraxes and heads were triturated with a mortar and pestle and suspended in medium RPMI 1640 containing 1% C57BL/6 mouse serum (Rockland Co, Gilbertsville, Pa., USA). 50-80 heads with salivary glands were placed into a 0.5 ml Osaki tube on top of glass wool with enough dissection media to cover the heads. Until all mosquitoes had been dissected, the Osaki tube was kept on ice. Sporozoites that were isolated from the same batch of mosquitoes were inoculated into C57BLJ 6, 2D knock-out and 2D knock-out/2D6 knock-in C57BL/6 mice on the same day to control for biological variability in sporozoite preparations. On day 0, each mouse was inoculated intravenously in the tail vein with approximately 10,000 sporozoites suspended in 0.1 ml volume. They were stained with a vital dye containing fluorescein diacetate (50 mg/ml in acetone) and ethidium bromide (20 μg/ml in phosphate buffered saline; Sigma Chemical Co, St. Louis, Mo., USA) and counted in a hemocytometer to ensure that inoculated sporozoites were viable following the isolation procedure. Viability of the sporozoites ranged from 90 to 100%.

Animals. The mice used in these experiments were albino C57BL/6 female mice, which were housed in accordance with the current Guide for the Care and Use of Laboratory Animals (1996) under an IACUC approved protocol. All animals were quarantined for 7 days upon arrival, and the animals were fed standard rodent maintenance food throughout the study.

Test compounds, homogenization of JAG21 creating a nanoformulation, and administration. Animals were dosed with experimental compounds based on body weight. The suspension solution of orally administered drugs were conducted in 0.5% (w/v) hydroxyethyl cellulose and 0.2% Tween 80 in distilled water. To insure the size of the compounds in the dosing solution were under 50 μM (measured they were 4-6 p), the suspension was homogenized using a homogenizer (PRO Scientific Inc., Monroe, Conn., USA) with a 10 mm open-slotted generator running at 20,000-22,000 rpm for 5 min in an ice bath. The compounds were made fresh each day and used immediately (always in <½ hour). Stability beyond that time was not determined. It was not anticipated that they would be stable beyond that time.

Compounds were administered on three consecutive days (−1, 0, +1) relative to sporozoite infection or a single dose on day 0. Drug suspensions were administered to mice by oral gavage using an 18 gauge intragastric feeder. For the 3-day dosing regimen, compounds were administered at 0.625 mg/kg and for the single dose regimen administered on day 0, compounds were administered at 2.5 mg/kg.

In vivo imaging. All of the in vivo bioluminescent imaging methods utilized have been described previously. Briefly, JAG21 was administered orally on days −1, 0 and 1 with respect to sporozoite inoculation. All inoculated mice were imaged using the Xenogen IVIS-200 Spectrum (Caliper Life Sciences, Hopkinton, Mass., USA) IVIS instrument at 24, 48 and 72 hours post-sporozoite infection. The bioluminescent imaging experiments were conducted by IP injection of the luciferase substrate, D-Luciferin potassium salt, (Xenogen, Calif. and Goldbio, St Louis, Mo., USA), into mice at a concentration of 200 mg/kg 15 min before bioluminescent images were obtained. Three minutes after luciferin administration the mice were anesthetized using isoflurane, and the mice were positioned ventral side up on a 37° C. platform with continual anesthesia provided through nose cone delivery of isoflurane. All bioluminescent images were obtained using 5 minute exposures with f-stop=1 and large binning setting. Photon emission from specific regions was quantified using Living Image®3.0 software (Perkin Elmer),

Additionally, blood stage parasitemia was assessed 3 days after imaging was completed by treating small quantities of blood obtained from tail bleeds with the fluorescent dye Yoyo-1 measured by using a flow cytometry system (FC500 MPL, Beckman Coulter, Miami, Fla., USA), (Pybus et al, 2013; Marcissin et al, 2014)

### Methods for Co-Crystallization and Binding Studies:

Bovine cytochrome bc₁ activity assays. Bovine cytochrome bc, inhibition assay was carried out in 50 mM KPi pH 7.5, 2 mM EDTA, 10 mM KCN, 30 μM equine heart cytochrome c (Sigma Aldrich), and 2.5 nM bovine cytochrome bc, at room temperature. 20 mM inhibitors dissolved in DMSO were added to the assay at a desired concentration without prior incubation. The working concentration of DMSO in the assay did not exceed 0.3% v/v. The reaction was initiated by the addition of 50 μM decylubiquinol (Abcam). The reduced cytochrome c was monitored by the different absorption between 550 and 542 nm using extinction coefficient of 18.1 mM⁻¹ cm⁻¹ in a SPECTRAmax Plus 384 UV-visible Spectrometer. The initial kinetic rate is determined as a zero-order reaction and used as the specific activity of cytochrome bc₁.

### Bovine Cytochrome Bc1 Purification Protocol.

Preparation of crude mitochondria: Whole fresh bovine heart was collected after slaughter and transported in ice. All work was carried out at 4° C. Lean heart muscle was cut into small cubes and homogenized in the buffer composed from 250 mM sucrose; 20 mM K₂HPO₄; 2 mM succinic acid; 0.5 mM EDTA. Buffer was added at a ratio of 2.5 L per 1 kg of muscle tissue. pH of resulting homogenate was adjusted to 7.8 using 2 M Tris and PMSF protease inhibitor was added to 0.1 mM concentration. The homogenate was then centrifuged in a Sorvall GS-3 rotor at 5000 g for 20 minutes. The resulting supernatant was then transferred to a Sorvall GSA rotor and centrifuged at 20,000 g for 20 minutes. Obtained mitochondrial pellet was washed in 50 mM KPi (pH 7.5); 0.1 mM PMSF buffer before second centrifugation under the same condition. The pellet was collected and sored at −80° C. for further use.

Solubilization of membrane proteins. The frozen mitochondria were thawed and re-suspended in 50 mM KPi (pH 7.5); 250 mM NaCl; 0.5 mM EDTA; 0.1 mM PMSF buffer; a small sample was taken for quantification of total mitochondrial proteins by BCA assay. The remaining sample was centrifuged at 180,000 g in Beckman Ti70 rotor for 60 minutes. The pellet was re-suspended in the same wash buffer with the addition 1 mg DDM per 1 mg of protein and then centrifuged under the same conditions for 60 minutes. The pellet was discarded and the supernatant was collected for ion exchange chromatography.

Purification of cytochrome bc1. During purification the presence of protein was detected using 280 nm absorbance and the presence of heme was detected using 415 nm Soret band peak and 562 nm absorbance. The solubilized protein solution was applied on DEAE-Sepharose CL-6B column (ca. 50 ml, GE Healthcare) pre-equilibrated with buffer A (50 mM KPi (pH 7.5); 250 mM NaCl; 0.01% w/v DDM; 0.5 mM EDTA) and washed with 3 CV of buffer A. The protein was eluted by linear gradient with buffer B (50 mM KPi (pH 7.5); 500 mM NaCl; 0.01% w/v DDM; 0.5 mM EDTA). Fractions containing cytochrome bc₁ were pooled and concentrated to 0.5 ml using an Amicon Ultra-15 (Amicon, MWCO 100,000) concentrator. Concentrated sample was applied to a Sephacryl-S300 gel filtration column (ca. 120 ml) pre-equilibrated in buffer C (20 mM KMOPS (pH 7.2); 100 mM NaCl; 0.01% w/v DDM; 0.5 mM EDTA) and eluted at a flow rate of 0.5 ml/min. Purified cytochrome bc₁ fractions were collected and concentrated to 40 mg/m. PEG fractionation with increasing concentration of PEG4000 was used to precipitate cytochrome bc. Precipitating solution (100 mM KMES pH 6.4; 10% PEG4000; 0.5 mM EDTA) was mixed with the protein to a desired PEG concentration. The precipitated protein pellet was re-solubilized in buffer D (25 mM KPi pH 7.5, 100 mM NaCl, 0.5 mM EDTA, 0.015% DDM) and dialyzed in the same buffer in a centrifugal ultrafilter to remove residual PEG. 5 μM cytochrome bc, was incubated at 4° C. for 12 hours with 50 μM JAC021 (10-fold molar excess) diluted from 20 mM solution stock in DMSO.

Crystallization, data collection and refinement of Cytochrome bc1-JAG021 complex. The inhibitor-bound cytochrome bc, was mixed with 1.6% HECAMEG to the final protein concentration of 40 mg/mL. Hanging drop methods was used for crystallisation. 2 μL of final protein solution with 2 μL of reservoir solution (50 mM KPi pH 6.8, 100 mM NaCl, 3 mM NaN₃, 10-12% PEG4000) was equilibrated over reservoir solution at 4° C. The crystals were grown to 100 μm within four days. The single crystal was transferred in reservoir solution containing increasing to 50% concentrations of ethylene glycol prior to cryo-cooling in liquid nitrogen. X-ray data were collected from single crystal PROXIMA2 beamline, SOLEIL light source, France using DECTRIS EIGER X 9M detector at 0.9801 Å wavelength up to 3.45 Å resolution. Data were indexed and integrated using iMosflm (Battye et al, 2011), and scaled using Aimless (Evans, 2011). The starting model for refinement was 5OKD. All ligands except co-factors were removed from the model prior to refinement. Jelly-body refinement was carried out with Refmac5 (Murshudov et al, 2011). The inhibitor model was generated by Jligand (Lebedev et al, 2012). The model was manually edited in COOT (between cycle refinements. Data collection and refinement statistics are shown in Table 2A.

TABLE 2A
Data collection and refinement statistics for bc1-JAG021

| | bc₁-JAG021 |
|---|---|
| Data collection | |
| Space group | P6₅22 |
| Cell dimensions | |
| a, b, c (Å) | 209.87, 209.87, 342.46 |
| α, β, γ (°) | 90°, 90°, 120° |
| Resolution (Å) | 90.88-3.45 (3.56-3.45) |
| R_{merge} (%) | 22.5 (111.0) |
| Rₚᵢₘ (%) | 6.8 (33.4) |
| I/σI | 8.6 (2.4) |
| Completeness (%) | 91.4 (92.8) |
| Redundancy | 11.4 (11.6) |
| Wilson B-factor (Å²) | 87.4 |
| Refinement | |
| Resolution (Å) | 90.88-3.45 |
| No. reflection | 48,681 |
| R_{work}/R_{free} | 21.74/23.24 |
| No. atoms | |
| Protein | 15,505 |
| Inhibitor | 30 |
| Water | 19 |
| Other ligands | 567 |
| B-factors | |
| Protein | 164.73 |
| Inhibitor | 143.91 |
| Water | 59.81 |
| Other ligands | 180.69 |
| R.m.s. deviations | |
| Bond length (Å) | 0.008 |
| Bond angle (°) | 1.426 |
| PDB code | 6XVF |

* data in brackets are for the last shell

### Co-Cryo Electron Microscopy:

Electron microscopy and image processing. Cryo-EM was carried out as described in Ampomdanai et al. (2018). Briefly, 3 μL of sample at 5 mg/mL concentration were applied to Quantifoil Cu R1.2/1.3, 300 mesh holey carbon grids and plunge frozen using an FEI Vitribot (blot time 6 seconds, blot force 6). Data were collected on an FEI Titan Krios with a Falcon III direct electron detector operated in integrating mode at 300 kV Automated data collection was carried out using EPU software with a defocus range of ˜1 to ˜3.5 Nm, and a magnification of 75,000× which yielded a pixel size of 1.065 Å. Data were collected for 72 hours resulting in 5,356 micrographs. The total dose was 66.4 e⁻/Å over a 1.5 second exposure which was split into 59 frames. All of the processing was performed in RELION 2.1 unless otherwise stated. The initial drift and CTF correction was carried out using MOTIONCORR2 (Zheng et al. 2017) and Gctf (Zhang et al. 2016), respectively. The micrographs were examined and those with crystalline ice were initially removed resulting in 2,960 micrographs. A subset of about 2,000 particles were manually picked to generate 2D references to facilitate auto-picking resulting in 439,009 particles. These particles underwent an initial round of 2D classification with those classes that displayed clear secondary structure detail being taken forward to 3D classification and split into three classes. Two of the three classes generated a high-quality cytochrome bc, reconstruction with secondary structure information clearly visible. The particles from these two classes were recombined to form the final datasets consisting of 211,916 particles in the final reconstruction. The particles were 3D refined using C2 symmetry to produce a map with resolution 3.8 Å. The particles also underwent movie refinement and particle polishing which further improved the resolution of the map to 3.7 Å. A previously refined EM structure for SCR0911 (pdb 6FO6) was fit into the map using UCSF chimera and subsequently refined using phenix with the correct ligand. The maps were then inspected manually in COOT (Emsley et al 2004) and the model corrected for any errors in refinement and placement of residues. Statistics are in Table 2B.

TABLE 2B
Data collection statistics for the cryoEM
reconstruction of cytochrome bc1.

| | bc₁-JAG |
|---|---|
| Detector | Falcon III |
| Detector mode | Integrating |
| Voltage (kV) | 300 |
| Pixel size (Å) | 1.065 |
| Defocus (μm) | −1 to −3.5 |
| Total dose (e/Å) | 66 |
| No. of frames | 59 |
| Exposure time (s) | 1.5 |
| Dose per frame | 1.12 |
| No. of micrographs | 5,356 |
| Total particle No. | 439,009 |
| Final particle No. | 211,916 |
| Resolution | 3.3 Å |

Statistical Analysis. A Pearson test was used to confirm a correlation between increasing dose and increasing inhibition. An ANOVA and subsequent pairwise comparison with Dunnett correction was used to determine whether or not inhibition or toxicity at a given concentration was statistically significant. Stata/SE 12.1 was used for this analysis.

### Example 3: Results

### THQ Compounds are Potent In Vitro:

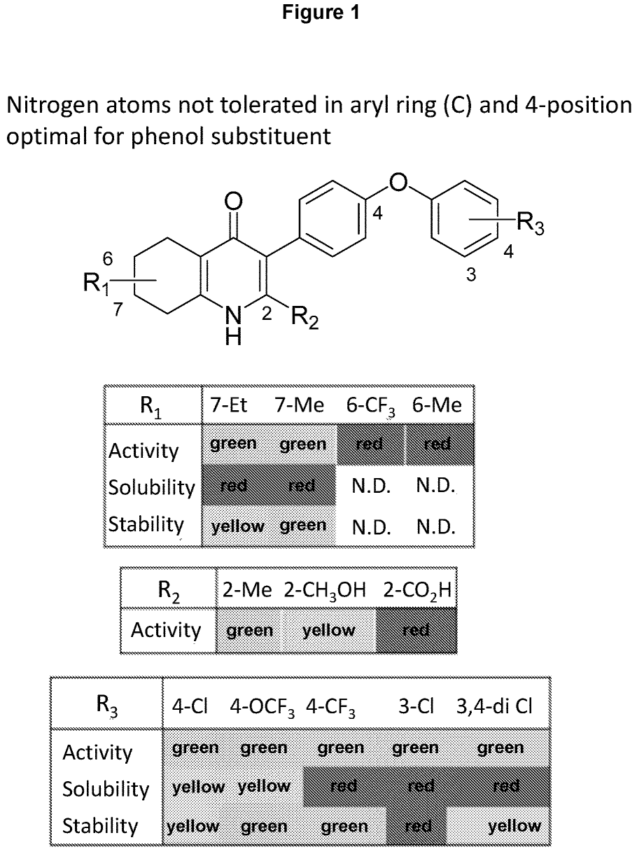

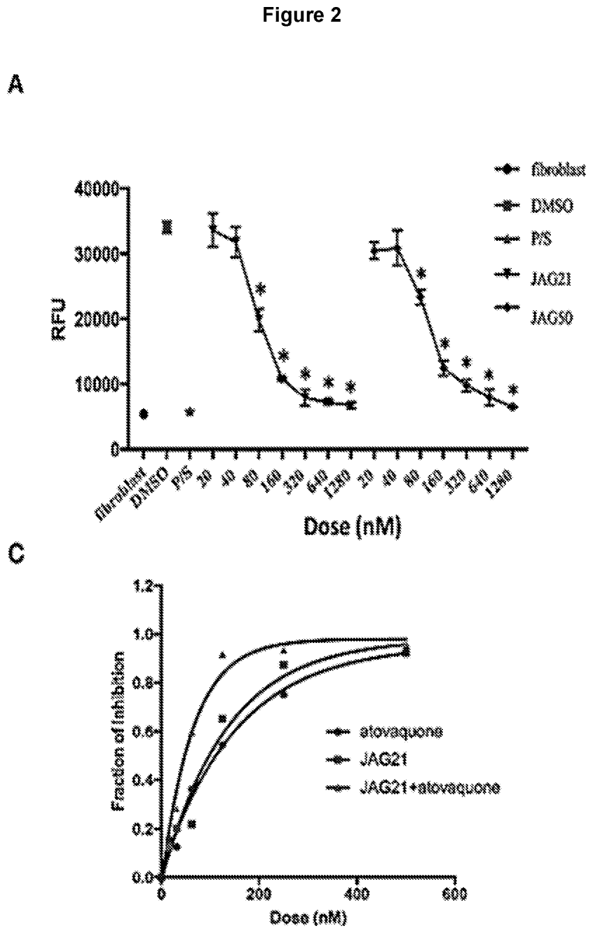

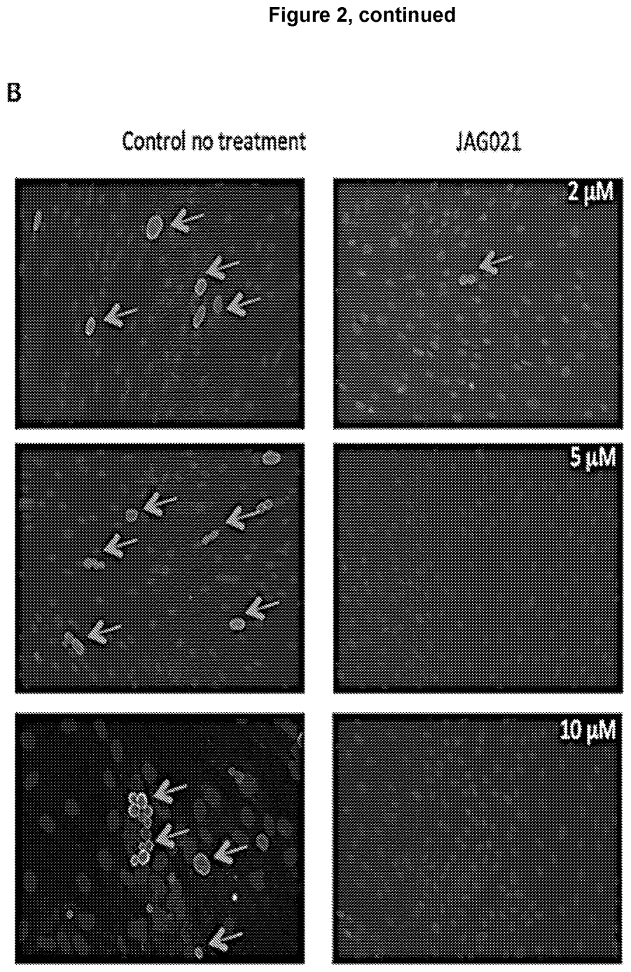

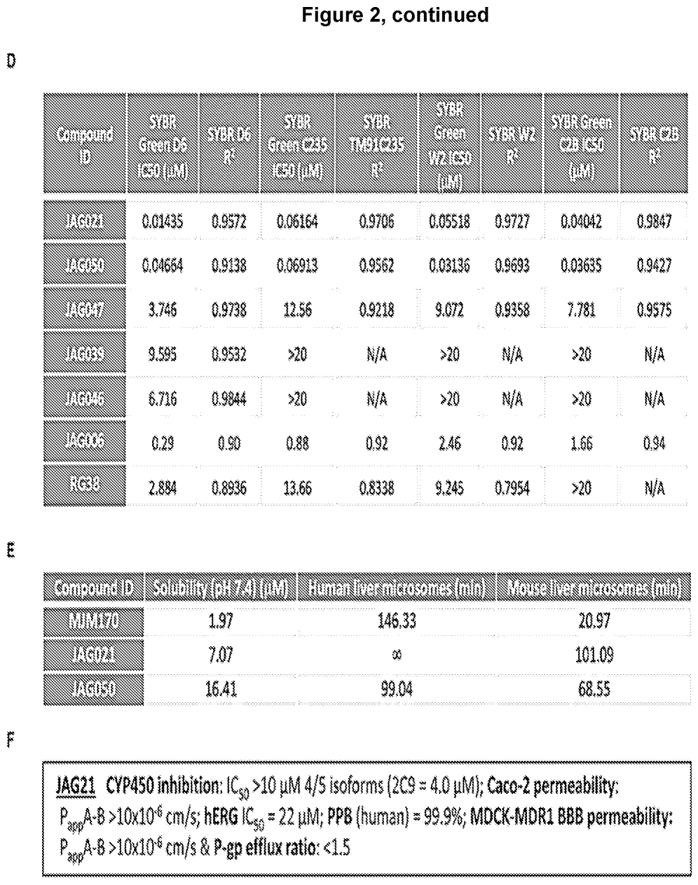

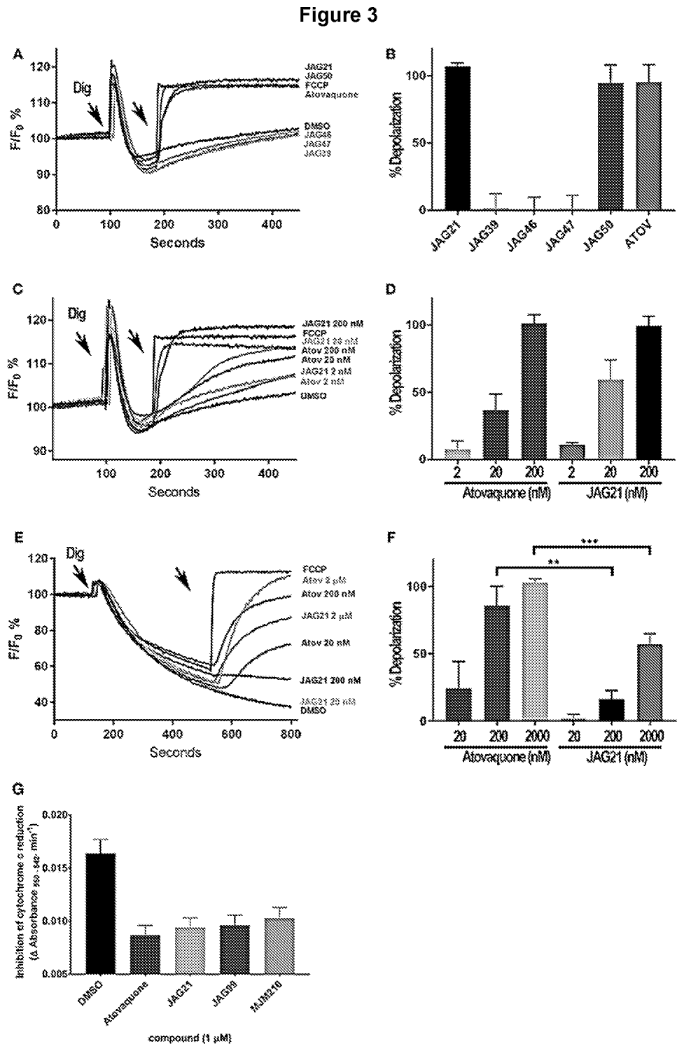

Initially, a small library of seven compounds (FIGS. 1 and 2) were tested, and each compound was tested at least twice against T. gondii tachyzoites. JAG021 and JAG050 demonstrated effect below 1 μM, and were tested at lower concentrations. JAG050 and JAG021 were identified as lead compounds given the IC₅₀ values obtained were 33 and 55 nM, respectively. Correlation between concentration of compound and inhibition of parasite replication (as measured by fluorescence) was observed for all compounds except JAG046. The relative effect on HFF and parasite enzymes were also compared, with those marked * in Table 1 having the most effect on the parasite enzyme activity relative to host HFF enzyme activity as shown below in FIG. 3.

A representative graph of these in vitro data is shown in FIG. 2A. Subsequently, a larger library of 54 compounds was synthesized to ascertain structure-activity relationships (SAR) (FIG. 1). The primary aims were to block putative metabolism of the terminal phenol ring of MJM170 and improve the solubility across the compound series. Substituents were generally tolerated at the meta and para positions on the phenol ring (R₁), similar to the trends observed in the ELQ series (McPhillie et al, 2016; Dogget et al 2012, Vidigal et al 2002). The incorporation of heteroatoms into the aryl rings of the biphenyl moiety did not lead to improvements in solubility and biological activity. Small substituents were tolerated at the 7-position of the THQ bicyclic ring (FIG. 1; R₁), improving selectivity (see below, SAR) but not at the 6-position unlike the ELQ series. In summary, overall, nitrogen atoms not tolerated in aryl ring (C) and the 4-position was optimal for phenol substituent. Ultimately, no other compound had all the advantages of JAG21, although some of these were identified as potential back up compounds (marked with *), with greater selectivity for the parasite relative to the mammalian enzyme activity. Compound JAG21 displayed synergy against RH strain tachyzoites with atovaquone (FIG. 2C) but not with pyrimethamine, although no antagonism was observed (data not shown).

Cytotoxicity assays performed in parallel using HFF, WST-1 (Fomovska et al. 2012; Fomovska et al. 2012a), and HEP G2 cells demonstrated a lack of toxicity at concentrations substantially in excess of the concentrations effective against tachyzoites. Because T. gondii grows inside cells, if a compound were toxic to host HFF then it would make the compound appear to be spuriously effective (Fomovska et al. 2012; Fomovska et al. 2012a), when in actuality only toxicity for the host cell would be measured. Cytotoxicity to HFF was therefore assessed for all compounds at 10 μM. Results of this experiment are in Table 1, toxicity column. A two-way ANOVA and subsequent pairwise comparison found none of the differences in absorbance, compared to the media-DMSO vehicle controls, to be statistically significant (p>0.05). Most of these compounds are not toxic at 10 μM (the limit of solubility) and that cytotoxicity to cells can be attributed to DMSO in the solution, not the compound. Dose response testing (IC₅₀) was performed with HEP G2 cells as described and the observed toxicity was: HEP G2 IC₅₀ 17.70 μM (r²=0.97) for JAG021; 7.1 μM (r²=0.98) for JAG050.

Lead compounds JAG050, JAG021 and others were tested against EGS strain (McPhillie et al, 2016; Vidigal et al, 2015; Paredes-Santos et al 2013; Paredes-Santos et al, 2018) tachyzoites and encysted bradyzoites using methods described earlier (McPhillie et al, 2016). A number of these compounds, including JAG21, were highly effective against tachyzoites (RH-YFP; Fomovska et al. 2012) (Table 1, FIGS. 2A,C) and bradyzoites of EGS (McPhillie et al, 2016; Vidigal et al. 2015; Paredes-Santos et al 2013; Paredes-Santos et al. 2018) (FIG. 2B). For example, in a separate experiment (data not shown) using immunofluorescence microscopy, the following forms were observed: “true cysts” with a dolichos-staining wall, “pseudocysts” or tight clusters of parasites, and small organisms. If there were fewer than four parasites visible in a cluster, organisms were counted individually (as “small organisms”). A statistically significant reduction in the number of true cysts and small organisms was observed at 1 μM and 10 μM for both compounds (p<0.05, p<0.005). 500 nM JAG21 treatment results in cultures where EGS bradyzoites were not seen (e.g., FIG. 2B).

Results against P. falciparum using methodology described earlier (McPhillie et al, 2016, Trager et al, 2005, Ploufe et al, 2008, Johnson et al, 2007) also are shown in FIG. 2D. JAG 21 is potent against P. falciparum with IC50 values ranging from 14-61 nM against a variety of drug sensitive and resistant strains (McPhillie et al 2016) including D6, TM91-C235, W2, and C2B. The D6 strain is a drug sensitive strain from Sierra Leone, the TM91-C235 strain is a multi-drug resistant strain from Thailand, the W2 strain is a chloroquine resistant strain from Thailand, and the C2B strain is a multi-drug resistant strain resistant to atovaquone. Effects of other comparison compounds are also shown in this table and range from 31 to 20,000 nM (FIG. 2D).

### ADMET Superiority of JAG21:

In vitro absorption, distribution, metabolism, excretion, and toxicity (ADMET) analyses of the THQ compounds were outsourced to ChemPartner Shanghai Ltd. ELQ-271 (synthesized in-house) was tested as a comparison. THQs which were potent inhibitors of T. gondii tachyzoites were assessed for their kinetic solubility, metabolic stability in human and mouse liver microsomes (FIG. 2E), hERG, and their ability to permeate across MDCK-MDR1 cell membranes (in vitro measure of blood-brain barrier (BBB) penetration potential/permeability). Solubility, half-life, HERG, and BBB permeability/efflux results are shown in FIG. 2F. The aqueous solubility (PBS, pH 7.4) of amorphous compounds JAG021 and JAG050 was 7 and 16 μM respectively, which is improved over MJM170 (2 μM) and ELQ-271 (0.2 μM). Solubility of the microcrystalline form of JAG21 was also tested, and it was found that the solubility was 3.5 μM. JAG021 was the most metabolically stable compound in human liver microsomes (>99% remaining after 45 minutes) compared with other THQs and ELQ-271, although it displayed a much shorter half-life of 101 minutes in mouse liver microsomes. All THQs tested in the MDCK-MDR1 system for blood brain barrier (BBB) permeability (including MJM170, JAG021 and JAG050), exhibited high permeability (Pₐₚₚ>10×10⁸ cm/s) and low efflux (efflux ratio<1.5).

### THQs Potently Inhibit Parasite Cytochrome Bc1 (Cytbc1) Enzyme Activity:

JAG21 is the most active of the initially tested THQs against T. gondii Cytbc1, which also showed selectivity for the parasite over the mammalian mitochondrial membrane potential (FIG. 3). Following the full SAR testing in vitro activity against tachyzoites, the full set of compounds was tested against HFF; then the initial compounds also were tested against the T.gondii and HFF enzyme benchmarked against atovaquone, and ultimately the full set of compounds was compared for effect against the T. gondii and HFF enzymes.

Mitochondrial membrane potential measurements were performed with permeabilized T.gondii tachyzoites in suspension using safranin O, which loads into polarized membranes. T. gondii tachyzoites were permeabilized with digitonin to allow the mitochondrial substrate succinate to cross the membrane and energize the mitochondrion. The fluorescence of safranin O, which loads into energized mitochondria was used to measure the membrane potential. The energized state of the mitochondrion is observed by a decrease in fluorescence (FIGS. 3A,C,E). Trifluoromethoxy carbonylcyanide phenylhydrazone (FCCP) was used to depolarize the membrane, which causes the fluorescence to go up as shown in FIG. 3A. JAG21 depolarized the membrane potential even at concentrations as low as 2 nM (FIG. 3C,D). JAG21 and Atovaquone had similar effects on the mitochondrial membrane potential (FIG. 3D). FIG. 3F is host cells so indicates that it is less toxic. Other compounds like JAG46 and 47 showed almost no effect at doses as high as 4 μM (FIG. 3A,B). JAG50 showed depolarizing activity at doses of 200 nM and higher. The effect of these THQ compounds against the T. gondii mitochondrial membrane potential was greater than the effect on the human foreskin fibroblast membrane potential (FIG. 3E,F). This is consistent with the observation that JAG21 is less toxic against human Telomerase reverse transcriptase immortalized (hTERT) HFF cells than atovaquone. Newly created THQ compounds that show even less toxicity to the human fibroblast cytochrome b/c complex are marked with * in Table 1. These could be developed in a second phase of the program were reductions in toxicity needed. However, as data presented herein demonstrates, there are significant advantages in the ADMET properties of JAG 21, and its dramatic efficacy in vivo, without toxicity. There may be no need to further develop any of those potential additional leads.

Enzyme reduction of cytochrome c by P. falciparum parasite extract (Fisher et al, 2004, 2009) is mediated by P. falciparum bc₁ complex cytochrome c reductase (Pfbc₁) enzyme. All three compounds tested (1 μM) significantly inhibited the reduction of cytochrome c by the P. falciparum parasite extract, (JAG021=86.4±3.2; JAG099=81.3±6.0; MJM170=69.7±11.3% of the atovaquone response, FIG. 3G. Additional data demonstrated selective effect on P. falciparum enzyme compared with bovine enzyme (data not shown).

### Binding, Co-Crystallography, Pharmacophore and Co-Cryo-Electron Microscopy Studies Demonstrate Selectivity:

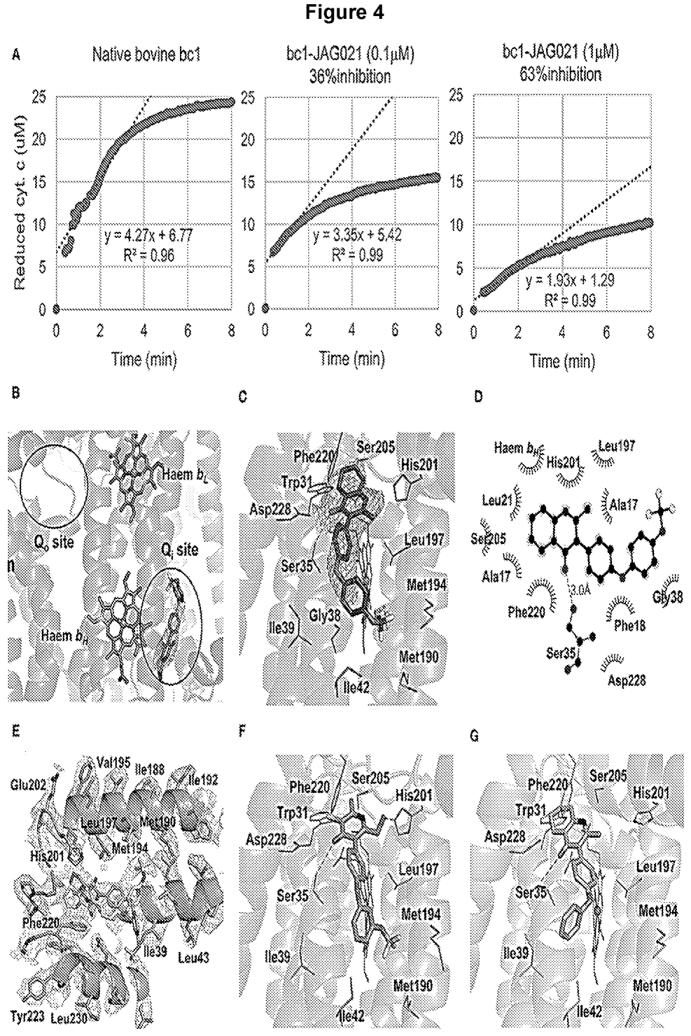

In binding assays and in co-crystallography (McPhillie et al, 2016; Capper et al, 2015; Ampomdanai et al, 2018; Battye et al, 2011; Emsley et al, 2010; Laskowski and Swindells, 2011; Lbebedev et al, 2012; Murshudov et al, 2011; Emsley et al, 2004; Zheng et al, 2017; Zhang et al, 2016), JAG021 has lower binding affinity to bovine cytochrome bc in comparison with previous compounds that have not been tested. JAG 21 ‘inhibits’ Cytbc1 but not fully, indicating that it will be less toxic for mammalian (bovine/human) cyt bc1 than the apicomplexan enzymes (FIG. 4A). The electron density map in the Qᵢ site of bovine cytochrome bc, complex with JAG021 (Table 2A, Data Collection Statistics) reveals an additional electron density, which allowed unambiguous positioning of the inhibitor (FIG. 4B). No additional electron density was found within the Qₒ site. After the refinement, 2Fₒ-F_{c} electron around JAG021 becomes clearer (FIG. 4C). The second aromatic ring in the tail group of the compound is less defined due to high flexibility introduced by the oxygen linker. The quinolone head of JAG021 is held between Asp228 and His201 and adapted the same conformation as 4(1H)-pyridone (GSK932121)(Capper et al, 2015) (FIG. 4D) and tetrahydro-4(1H)-quinolone (MJM170) (McPhillie et al, 2016) (FIG. 4E) by directing the NH group to His201 and the carbonyl group to Asp228. The carbonyl of the quinolone head and OG1 atom of Ser35 are within 3.0 Å distance that allows hydrogen bonding and enhances the binding affinity to the bovine enzyme. The 3-diarylether tail extends along a hydrophobic channel defined by Gly38, Ile39 and Ile42. The trifluoromethoxy group at the phenoxy ring points towards Met190 and Met194 (FIG. 4F). CryoEM studies of the complex also demonstrates reasons for selectivity. In FIG. 4F, the density suggests that the inhibitor can adopt two different binding poses as observed previously in the cryo-EM structure of GSK932121(Capper et al, 2015). The binding pose shown in yellow, which has the strongest density, agrees with the crystal structure and has the trifluoromethoxy group pointing towards Met194. However, there is additional density which could result from a second binding pose (green) in which the trifluoromethoxy group points towards Asp228(McPhillie, 2016 et al). FIG. 4F, shows GSK932121 pyridone (PDB:4D6U) (G) MJM170 quinolone (PDB:5NMI).

### JAG21 is Potent In Vivo:

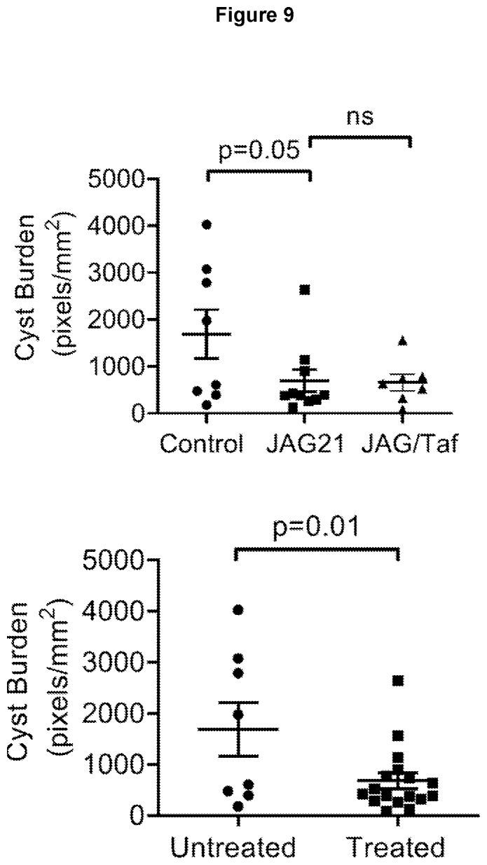

In vivo studies of JAG21 against T. gondii demonstrated high efficacy in a variety of settings. JAG21 at 5 mg/kg/day administered IP improves well-being and eliminates illness and T. gondii Type II Prugneaud luciferase tachyzoites completely in luminescence studies (FIG. 5A). Further, treatment beginning on day one after infection results in no cysts being found in brains of these mice treated for 14 days with 5 mg/kg/day of JAG21, when brains were evaluated 30 days after stopping JAG21 treatment in two replicate experiments. Treatment beginning on day 30 after initiation of infection with Type II Me49 parasites results in marked, statistically significant reduction in normal appearing cysts, free organisms and immunoperoxidase stained cysts detected by automated imaging of scanned slides (FIGS. 5 B,C, p<0.03 experiment; p<0.01 experiments 1 and 2 together, FIG. 9). The automated analysis confirmed results from the blinded microscopic visual quantitation of cysts and free organisms in slides by two observers. Adding tafenoquine or primaquine to treatments of active plus dormant malarias (St. Jean et al, 2016; Lacerda et al, 2019; Llanos-Cuentas et al 2019) is partially effective against both active and dormant phase plasmodia, when neither treatment of active nor dormant disease alone is effective for either in vivo. Experiments based on these observations were developed where experiments with tafenoquine alone or with JAG21 alone was used in the experiments with established cyst with immune competent mice. This was to determine whether tafenoquine might add to efficacy of JAG21. The efficacy of treatment with JAG21 alone was so robust (FIG. 5B), that no additive effect was seen, or could have been detected, by adding Tafenoquine to JAG21. Efficacy was shown when data were analyzed as separate groups, i.e., control vs JAG21 alone (p<0.03) or control vs JAG21 plus tafenoquine, or grouping the JAG21 and JAG21 plus tafenoquine results as “untreated” versus “treated” (p<0.01). Analysis shown combining both treatment groups from two replicate experiments showed similar results (p<0.01) (FIG. 5B), and when results from replicate experiments were grouped (FIG. 9). In FIG. 5C the control mice had cysts with usual morphology (Top two panels), whereas treated mice had very few morphologically recognizable usual cysts that were immunostained (bottom panels).

A nano formulation homogenized (<6 μM) was used effectively orally for the P.bergheii experiments, further, importantly, was effective in the single oral dose causal prophylaxis in 5 C57BL/6 albino mice at 2.5 mg/kg dosed on day 0, 1 hour after intravenous administration of 10,000 P. berghei sporozoites was completely protective. In addition, 3 dose causal prophylaxis treatment in 5 C57BL/6 albino mice at 0.625 mg/kg dosed on days −1, 0, and +1 also was completely protective. A representative experiment at a higher dose (5 mg/kg) is shown, but all experiments with the oral dosing regimen with the nanoformulation specified above showed 100% survival 30 days post infection with P. berghei, where all liver and blood stage parasites were eliminated (FIG. 5D,E) demonstrates not only efficacy of JAG21 against the three life cycle stages of P. berghei, but also demonstrates the efficacy of oral administration of the nanoformulation when used immediately, at a low dose.

### G1 Arrest, Persisters, Companion Compounds:

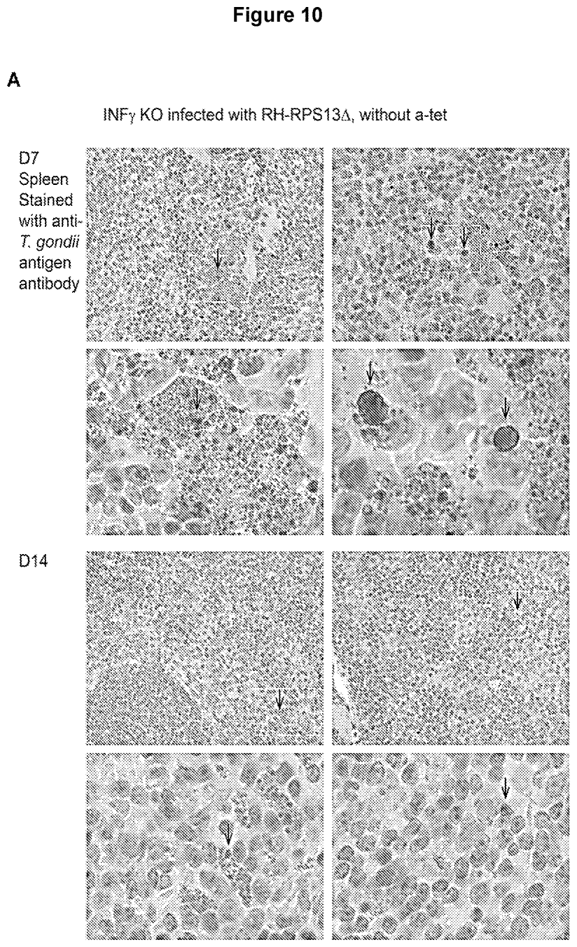

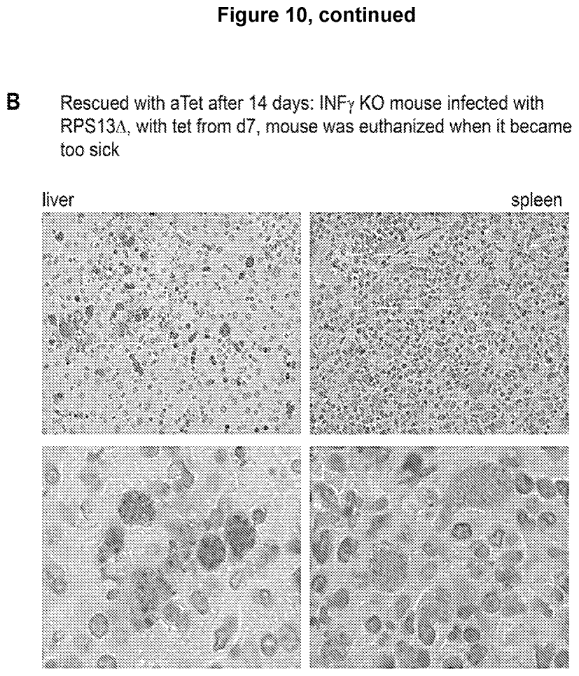

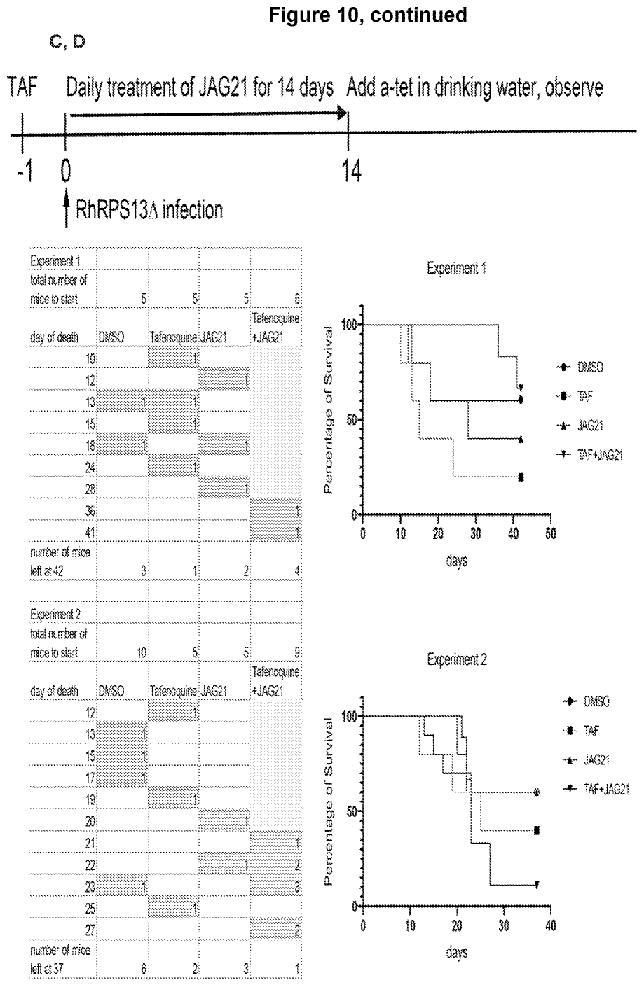

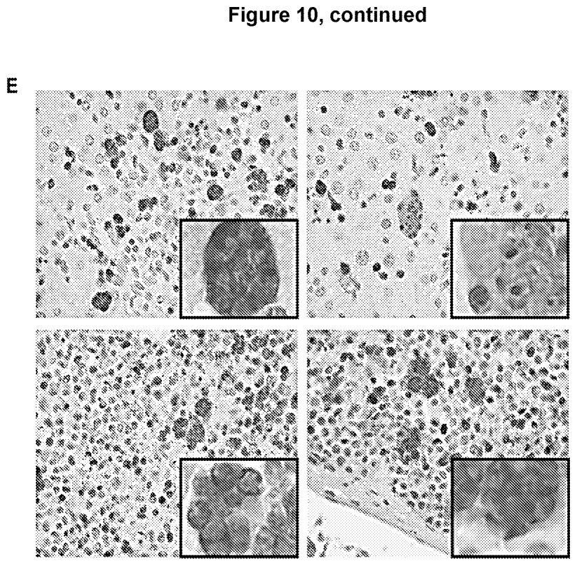

In mice that were treated with JAG21 early after infection (FIG. 5A), no residual immunostaining for T.gondii in brain tissue of any mice was found. This suggests that very early treatment could prevent established, chronic infection, for example in epidemics such as those that occurred in Victoria, Canada, the U.S.A., and Brazil. In mice with established cysts, following treatment with JAG21, occasionally a small number of cysts (FIG. 5B) and amorphous immunostained structures was observed (FIG. 5C bottom panels). This was reminiscent of persistence in some malaria infections (Cubi et al, 2017) and abnormal immunostained structures that were previously identified with a conditional, tetracycline-on regulatable, mutant T.gondii (Hutson et al, 2010 and FIG. 10). In this ΔRPS13 tachyzoite, small ribosomal protein 13 can be regulated, depending on whether anhydrotetracycline (ATc) is absent or present, leading the ATc responsive repressor to be on or off response elements engineered into the promoter (Hutson et al, 2010). ΔRPS13 replicates with ATc present and is arrested in G1 when ATc is absent in HFF cultures (Hutson et al 2010). The dormant parasite could persist for extended periods (Hutson et al, 2010). The parasite could be rescued from its dormant—ATc state by adding ATc, months after removing tetracycline from infected HFF cultures, although it could not be rescued in immunocompetent mice with LNAME and ATc when tested one week after infection (Hutson et al 2010). We wondered if this type of dormant organism could form in vivo, whether it could contribute in a biologically relevant way to dormancy and recrudescence, similar to the malaria hypnozoite, and whether JAG 21 might be able to eliminate it, or whether a companion compound effective against this form might be needed or work in conjunction with JAG21 if needed. To begin to address these questions and to investigate how close the T. gondii ΔRPS13-ATc phenotype might be to the malaria hypnozoite, the transcriptome of T. gondii ΔRPS13 in human, primary, brain, neuronal stem cells±ATc was compared to the recently published P. cynomolgi hypnozoite transcriptome-established with single cell RNA sequencing in laser captured organisms (Cubi et al, 2017). This analysis identified 28 orthologous genes with similar expression pattern in both T. gondii ΔRPS13-ATc and P. cynomolgi hypnozoites, including the downregulation of rps13 and upregulation of the eukaryotic initiation factor-2a kinase IF2K-B, a protein involved in translational control in response to stress (Cubi et al 2017) (FIG. 6A). Further. assessment of the T. gondii ΔRPS13 transcriptome in the absence or presence of ATc showed upregulation of additional IF2K members, 25 Apetela (AP) 2 transcription factors and a number of genes that participate as protein ubiquitin ligases, and in trafficking as well as in RNA binding (FIG. 8; Table 3). None of them, except for AP2VIIa-7, have been shown to be upregulated or downregulated during differentiation to bradyzoites. Gene set enrichment analysis showed that in the absence of ATc, the T. gondii ΔRPS13 transcriptome is enriched in genes typically expressed during G1, confirming previous results indicating that downregulation of rps13 arrests the parasite at this stage of the cell cycle (FIG. 6B) (Hutson et al, 2010). Moreover, a number of biological processes are downregulated without ATc, including protein synthesis and degradation as well as energy metabolism (FIG. 6B). Noteworthy, some gene ontology (GO) terms enriched in T. gondii ΔRPS13-Tc are also overrepresented in the P. cynomolgi hypnozoite (stars in FIG. 6D). Further, without ATc the transcriptome of T. gondii ΔRPS13 is compatible with a parasite transitioning from an active replicating form to a dormant stage, reflected by the downregulation of genes typical of the S and M stages of the cell cycle, and of genes that participate in energy metabolism and virulence (FIG. 6B, D, and FIG. 8; Table 3 and FIG. 10). It has been reported that with treatment of active forms of malaria, hypnozoites still persist, and recrudesce later (Cubi et al, 2017, Hutson et al 2010; St. Jean et al, 2016; Lacerda et al, 2019; Llanos-Cuentas et al, 2019). Also, compounds that target cytochrome b/c were not effective against malaria hypnozoites. If primaquine or tafenoquine, which do not treat the active P.vivax parasites, were added in vivo, hypnozoites have been shown not to recrudesce, or do so less often (St. Jean et al, 2016; Lacerda et al, 2019; Uanos-Cuentas et al, 2019). Testing with primaquine or tafenoquine could only be performed in vivo, as activity against the hypnozoite requires hepatic metabolism of primaquine or tafenoquine (St. Jean et al, 2016; Lacerda et al, 2019; Uanos-Cuentas et al, 2019). Tafenoquine is not active in tissue culture, which is consistent with the findings that these compounds require hepatic metabolism. To establish a parallel in vivo system, immune compromised mice (Interferon T receptor knockout mice with the knockout in the germline) infected with ΔRPS13 herein was studied. Although in immune competent mice ΔRPS13 does not recrudesce with ATc treatment beyond 3 days after infection, it was found that when ATc was added after treatment of the immune compromised mice with JAG21 dosed intraperitoneally for 14 days, the dormant ΔRPS13 parasite could still recrudesce after JAG21 treatment was discontinued and tetracycline added (FIG. 6C, FIG. 10). Consistent with adding tafenoquine to treatment of P.vivax malaria with chloroquine where both medicines together were partially effective against the active and hypnozoite forms, the combination of JAG21 and tafenoquine had a modest effect together on transiently improving survival time when ATc was added when compared with JAG21 or tafenoquine alone (FIGS. 6C, 10). The trend in the result seems similar to the malaria infections where hypnozoites form, although protection was not as robust, as in the malaria model, and it did not achieve complete, durable protection against ΔRPS13. These results in FIGS. 6C and 10 suggest: a. In G1 arrested organisms that begin as tachyzoites, they can persist in vivo even if their morphology as parasites is difficult to discern; b. Treatment with JAG21+Tafenoquine can prolong time to death more robustly than other treatments; c. But, in these immune compromised mice at this dosage regimen this treatment did not robustly, durably protect these mice from death later; d. In these immune compromised mice, whether this lack of complete protection was because of immune compromise, or less than optimal duration of treatment, or suboptimal dose or timing of treatments, or that this G1 arrested organism is harder to treat, remains to be determined in future studies. The modest efficacy of the two compounds, administered together, suggests that treating both tachyzoites and the G1 arrested organisms is important. This seems similar to P. cynomogli and P. vivax treatment with tafenoquine and chloroquine studies, which also showed efficacy but was not completely successful in preventing relapse. At the time this study was performed, formulation and dosing (including duration and timing) had not yet been optimized formally for the T.gondii model. P. vivax treatment requires chloroquine to treat blood schizonts and tafenoquine to treat hypnozoites. Treatment in man, per the FDA approved label, consists of a single dose of 300 mg on day 1 co-administered with chloroquine treatment on days 1 or 2. Both medicines have long half-lives in humans. This treatment was relatively effective in humans, with about a 30% recurrence rate.

Sinai et al have demonstrated heterogeneity in the phenotypes of organisms within established cysts. Their work found bradyzoites within cysts are not uniform with regard to their replication potential (Watts et al, 2015), mitochondrial activity (Sinai, unpublished), and levels of the glucose storage polymer amylopectin (Sinai, unpublished). These properties of bradyzoites within (Watts, et al, 2015), and properties of tissue cysts vary during the course of infection with unappreciated levels of complexity in the progression of chronic toxoplasmosis (Watts et al, 2015). The analysis (FIG. 6D) of the ΔRPS13 infected NSC suggests molecular targets modified in this G1 arrested ΔRPS13 parasite as shown in FIGS. 6D and 8; Table 3. In the future, with formulation and pharmacokinetics of JAG21 optimized, it will be of interest to determine whether JAG21 can eliminate these organisms and any residual structures as in FIG. 6C, or whether adding synergistic compounds such as atovaquone (FIG. 4B) or antisense effective against these upregulated molecular targets, such as kinases, ATPases, AP2s (FIG. 6D and Table 3), or a newly recognized bradyzoite master regulator of differentiation might be effective alone or might be synergistic with JAG21 against this ΔRPS13, as well as the conventional recognized tachyzoite and bradyzoite life cycle stages. Bradyzoites within tissue cysts are not monolithic, so in future studies single cell RNA sequencing of bradyzoites obtained by laser capture of bradyzoites in vivo defined on the basis of their physiological state, may be needed to determine whether a transcriptome signature similar to ΔRPS13 is sometimes present, linked to morphologic/immunostaining features that might functionally distinguish them to define the character of a hypnozoite-like state in T.gondii. Hererogeneity of parasite phenotype, even in the same vacuoles, in the earlier IFA and electron microscopic characterization of G1 arrested ΔRPS13 in HFF was noted (Hutson et al, 2010). Heterogeneity also was found very recently in tachyzoites and bradyzoites created by alkaline conditions in culture across the cell cycle in vitro in HFF, using single cell RNA sequencing (Xue et al., Biorx, Jun. 3, 2019 In Press). These authors also noted that what had been interpreted as “noise” earlier was found actually to be signal in a more complex environment. These authors suggest that such heterogeneity might make developing curative treatments more complex. The analysis of JAG21 effects and the ΔRPS13-ATet knockdown herein begin to help address this question: consistent with heterogeneity in the IFAs, in the comparison with the Xue et al.'s heterogeneous P1-6 clusters analysis, it was found that most of the up- or down-regulated genes are within P3-P5 tachyzoite clusters. Also, consistent with the heterogeneity observed in the G1 arrested ΔRPS13-ATet comparison, ΔRPS13 has a drop in SAG1 and elevated SRS44 that is consistent with a brady-like phenotype. BAG1 expression was too low overall to draw any conclusion about BAG1. It is also noteworthy that in the −ATet relative to +Atet conditions in primary, human, brain, neuronal stem cells, the master regulator of bradyzoite differentiation is slightly overexpressed (Log₂ Fold Change=0.7, adjusted p-value=0.043). Although JAG21 is highly potent against tachyzoites and bradyzoites, it did not eliminate every long-established encysted bradyzoite or -ATet ΔRPS13 completely either in vitro or in IFNγ knock-out mice in vivo. Consistent with heterogeneity, herein JAG21 treatment of ΔRPS13 and transcriptomics analyses define a metabolically quiescent, persister,“stasis” state that is reversible even after substantial periods of dormancy, that contribute to conceptual and functional understanding of both Plasmodia species and T. gondii infections and molecular mechanisms whereby “persisters” might be eliminated.

### An Oral Nanoformulation is Potent Against Virulent RH:

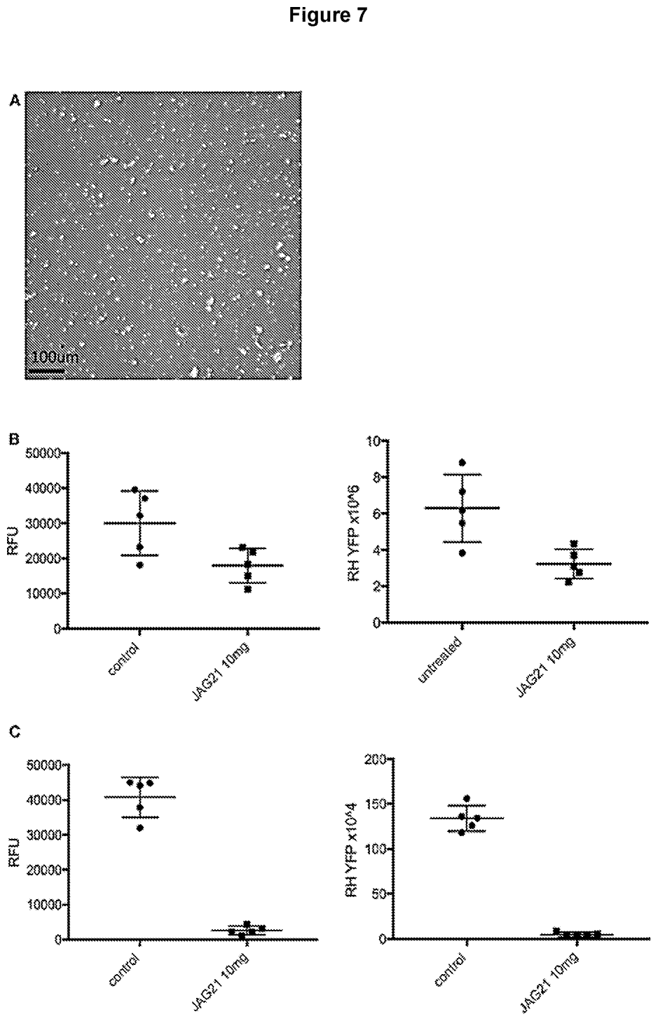

To further develop JAG21 for practical and clinical use, the next step was to make a formulation that is stable at room temperature, and would be effective when administered orally: Following a number of unsuccessful alternative methods (data not shown, a dispersion of JAG21 was prepared using hydroxyethyl cellulose (HEC) and Tween 80. This new formulation method is described in the Materials and Methods. When this dispersion was imaged using a Nikon ECLIPSE E200 optical microscope set to 40× magnification, the average particle size of the JAG21 dispersion in HEC/Tween 80 was determined using an in-house image analysis program and was found to be 2.85 μm (FIG. 7A). Material was re-sonciated the same way just prior to administration after being stored for 6 months and retained the same properties (FIG. 7A) when imaged. Following administration of 2,000 highly virulent RH Strain tachyzoites intraperitoneally, the oral nanoformulation was administered by gavage using a 21 gauge needle. This was given either (1) as a single dose of 5, 10, or 20 mg/kg, or (2) three daily doses of 10 mg/kg given for the first three days after infection. After 5 days the RH strain tachyzoites in peritoneal fluid of each mouse were quantitated by measurement of YFP they expressed using a flurimeter and by quantitating parasites present in peritoneal fluid using a hemocytometer. Parasite burden was reduced by about 60% 5 days later following the single doses of 10 and 20 mg/kg (representative experiment with 10 mg/kg shown in FIG. 7B; p<0.03) and markedly reduced with three doses of 10 mg/kg administered on each of the first three days after intraperitoneal injection of the virulent RH strain tachyzoites (FIG. 7C, representative experiment, p<0.001). This is the proof of principle that will facilitate media milling, dispersant, and a self-disintegrating tablet in the future. JAG21 has real promise as a mature lead compound to treat both T.gondii and Plasmodium species infections.

T.gondii infections are highly prevalent and the impact of this disease can be devastatingly severe. Current treatments have toxicity or hypersensitivity side effects. New compounds that are without toxicity or hypersensitivity, and that are highly active against tachyzoites would be of considerable clinical usefulness. Further, no medicines are active against the encysted stage or definitively curative. In addition, malaria is lethal for one child every eleven seconds and a threat to travelers going to endemic areas. Development of drug resistance also increases the need for new anti-malarial compounds. The disclosure herein identified compounds highly effective against T.gondii and P. falciparum with dual activity.

To further develop the THQ series, 54 compounds were synthesized to improve kinetic solubility, solubility in physiologically-relevant media (FaSSIF, FeSSIF), and metabolic stability (microsomes and hepatocytes), and other ADMET properties. Compounds JAG050 and JAG021 were identified as lead compounds, demonstrating potent inhibition on both tachyzoites and bradyzoites life stages and were not toxic to human cells in the in vitro model (HFF). In addition, both compounds displayed low nanomolar efficacy against multiple drug resistant strains of P. falciparum in vitro. JAG050 and JAG021 demonstrate promising ADMET properties, with JAG21 slight superior due to the compound's longer metabolic stability in human and mouse microsomes.

A striking result with JAG21 in the in vivo parasite studies is the compound's high efficacy against T.gondii tachyzoites and bradyzoites. In the P. berghei in vivo model for malaria, a single dose causal prophylaxis in 5 C57BL6 albino mice at 2.5 mpk dosed on day 0, 1 hour after intravenous administration of 10,000 P. berghei sporozoites was achieved. Causal prophylaxis was also observed after a 3-dose treatment in 5 C57BL/6 albino mice at 0.6 mpk dosed on days −1, 0, and +1. A representative figure at a higher dose (5 mg/kg) is shown, and all experiments with the amounts mentioned above demonstrated identical high efficacy in luminescence, parasitemia, and survival results. This demonstrates that JAG21 functions better in this in vivo model than the ELQ 300 series where prodrug formulation is required to achieve solubility and efficacy. In contrast to the efficacy of JAG21, at 2.5 mg/kg in a single oral dose model resulted in cure without a prodrug. ELQ 300 (not the prodrug) was not effective at doses between 1 and 20 mg/kg although the prodrug was more effective.

JAG050 and JAG021 are lead compounds, with JAG21 being a superior compound due to its favorable predicted ADMET properties, potency, efficacy and lack of toxicity. JAG021 demonstrates increased solubility and potential for advanced formulation. There also is potential for improving solubility and reducing toxicity further because of the larger binding pocket in the apicomplexan Cytbc1 enzyme compared with the mammalian Cytbc1 enzyme, determined by modeling occupancy of the structure, enzyme assays and empirically, if it were needed. At present, JAG21 has sufficient drug like properties to move to advanced formulations, suggesting increased bulk will not be needed to reduce toxicity. It has selectivity as demonstrated by the enzymatic, binding and structure studies, although there are additional compounds that show even greater selectivity. It is highly effective in an oral nano preparation against P. berghei three life cycle stages, and with early treatment appears to be capable of curing toxoplasmosis in immunocompetent mice. This work demonstrates the promising nature of this novel tetrahydroquinolone scaffold and mature lead compound. JAG21 has the potential to become an orally administered medicine or with partners, part of a medicine combination that is curative for toxoplasmosis and is a single dose cure for malaria. It is suitable for partnering with other compounds to obviate problems with selection of resistant mutants. In certain embodiments, the compounds of the disclosure may be combined with atovaquone and/or cycloguanil (in proguanil) against P. falciparum. The compounds of the disclosure are suitable for treatment of T. gondii or P. falciparum infections.

JAG21 treats both T.gondii and Plasmodium species infections, demonstrated in vitro and in vivo. It has high efficacy against T.gondii tachyzoites and bradyzoites, and established encysted organisms. Treatment with a single low oral dose is effective for causal prophylaxis and radical cure of P.berghei infections. JAG 21 has complete efficacy against three life cycle stages of P.berghei. In terms of companion inhibitors, JAG21, a Qᵢ inhibitor, synergizes against tachyzoites with atovaquone (a Qₒ inhibitor) in vitro. It appears able to contribute modestly to protection of immune compromised mice in conjunction with tafenoquine against an initially replicating, then G1 arrested, T.gondii parasite that shares key transcriptomic components with P. cynomolgi hypnozoites.

### REFERENCES

1. Anders S, Pyl P T, Huber W. (2015) HTSeq-a Python framework to work with high-throughput sequencing data. Bioinformatics. 31(2):166-9.
2. Ampomdanai K, Johnson R M, O'Neill P M, Fishwick C W G, Jamson A H, Rawson S et al., (2018). X-ray and cryo-EM structures of inhibitor-bound cytochrome bc 1 complexes for structure-based drug discovery. IUCrJ, 5, 200-210.
3. Battye T G, Kontogiannis L, Johnson O, Powell H R, Leslie A G. (2011) iMOSFLM: A new graphical interface for diffraction-image processing with MOSFLM. Acta Crystallographica Section D: Biological Crystallography, 67(4), 271-281.
4. Caumes E, Bocquet H, Guermonprez G, Rogeaux O, Bricaire F, Katiama C, et al. (1995) Adverse cutaneous reactions to pyrimethamine/sulfadiazine and pyrimethamine/clindamycin in patients with AIDS and toxoplasmic encephalitis. Clin Infect Dis Off Publ Infect Dis Soc Am. 21(3): 658-8.
5. Capper M J, O'Neill P M, Fisher N, Strange R W, Moss D, Ward S A, et al. (2015). Antimalarial 4(1H)-pyridones bind to the Qi site of cytochrome bc1. Proceedings of the National Academy of Sciences, 112(3):755-760.
6. Chen L F, Han X L, Li F X, Yao Y Y, Fang J P, Liu X J, et al (2018) Comparative studies of Toxoplasma gondii transcriptomes: insights into stage conversion based on gene expression profiling and alternative splicing. Parasites and Vectors 11:402
7. Croken M M, Qiu W, White M W, Kim K. (2014) Gene Set Enrichment Analysis (GSEA) of Toxoplasma gondii expression datasets links cell cycle progression and the bradyzoite developmental program. BMC genomics. 15:515.
8. Cubi R, Vembar S S, Biton A, Franetich J F, Bordessoulles M, Sossau D, et al (2017) Laser capture microdissection enables transcriptomic analysis of dividing and quiescent liver stages of Plasmodium relapsing species. P cynomolgi Cell Microbiol. 19(8).
9. Emsley P; Cowtan K (2004). Coot model building tools for molecular graphics. Acta Cryst. D60, 2126-2132
10. Emsley P. Lohkamp, B. Scott, W. G. Cowtan, K. (2010). Features and Development of Coot. Acta Crystallographica. D66: 486-501.
11. Evans, P R., (2011). An introduction to data reduction: Space-group determination, scaling and intensity statistics. Acta Crystallographica Section D: Biological Crystallography, 67(4), 282-292.
12. Fisher N. Warman A. Ward S A. Biagini G A (2009). Chapter 17 Type II NADH: Quinone Oxidoreductases of Plasmodium Falciparum and Mycobacterium Tuberculosis: Kinetic and High-Throughput Assays. Methods in Enzymology, ed. Mitochondrial Function, Part A: Mitochondrial Electron Transport Complexes and Reactive Oxygen Species. 456: 303-320.
13. Fisher N, Castleden C K, Bourges I, Brasseur G, Dujardin G, Meunier B (2004). Human disease-related mutations in cytochrome b studied in yeast. The Journal of biological chemistry, 279(13), 12951-12958.
14. Fomovska A, Wood R D, Mui E, Dubey J P, Ferreira L R, Hickman M R, et al. (2012) Salicylanilide inhibitors of Toxoplasma gondii. J Med Chem. 55(19): 8375-91
15. Fomovska A, Huang Q, El Bissati K, Mui E J, Witola W H, Cheng G, et al. (2012a) Novel N-Benzoyl-2-Hydroxybenzamide Disrupts Unique Parasite Secretory Pathway. Antimicrob Agents Chemother [Internet]. 56(5):2666-82.
16. Frueh L, Li Y, Mather M, Li Q, Pou S, Nilsen A, et al (2017). Alkoxycarbonate Ester Prodrugs of Preclinical Drug Candidate ELQ-300 for Prophylaxis and Treatment of Malaria. ACS Infect. Dis., 3(10):728-735
17. Gubbels M-J, Li C, Striepen B. (2003) High-Throughput Growth Assay for Toxoplasma gondii Using Yellow Fluorescent Protein. Antimicrob Agents Chemother 47(1):309-16
18. Hutson S L, Mui E, Kinsley K, Witola W H, Behnke M S, El Bissati K, et al (2010) T. gondii RP promoters & knockdown reveal molecular pathways associated with proliferation and cell-cycle arrest. PLoS One. 5(11):e14057.
19. Johnson J D, Dennull R A, Gerena L, Lopez-Sanchez M, Roncal N E, Waters N C. (2007) Assessment and Continued Validation of the Malaria SYBR Green I-Based Fluorescence Assay for Use in Malaria Drug Screening. Antimicrob Agents Chemother 51:1926-1933.
20. Kim D, Langmead B, Salzberg S L. (2015) HISAT: a fast spliced aligner with low memory requirements. Nature methods. 12(4):357-60.
21. Khan A A, Nasr M, Araujo F G. (1998) Two 2-hydroxy-3-alkyl-1,4-naphthoquinones with in vitro and in vivo activities against Toxoplasma gondii. Antimicrob Agents Chemother. 42(9): 2284-9.
22. Lacerda M V G, Uanos-Cuentas A, Krudsood S, Lon C, Saunders D L, Mohammed R, et al. (2019) Single-dose Tafenoquine to Prevent Relapse of Plasmodium vivax Malaria. N Engl J Med 380: 215-28.
23. Laskowski, R A. Swindells, M. B., (2011). LigPlot+: Multiple Ligand À Protein Interaction Diagrams for Drug Discovery. Journal of Chemical Information and Modeling, 51: 2778-2786.
24. Lebedev, A A et al., (2012). JLigand: A graphical tool for the CCP4 template-restraint library. Acta Crystallographica Section D: Biological Crystallography, 68(4): 431-440.
25. Uanos-Cuentas A, Lacerda M V G, Hien T T, Vélez I D, Namaik-Larp C, Chu C S, et al. (2019) Tafenoquine versus Primaquine to Prevent Relapse of Plasmodium vivax Malaria N Engl J Med 380:229-41
26. Love M I, Huber W, Anders S. (2014) Moderated estimation of fold change and dispersion for RNA-seq data with DESeq2. Genome biology. 15(12):550.
27. Marcsisin S R, Sousa J C, Reichard G A, Caridha D, Zeng Q, Roncal N, et al (2014) Tafenoquine and NPC-1161B require CYP 2D metabolism for anti-malarial activity: implications for the 8-aminoquinoline class of anti-malarial compounds. Malar J. 13:2.
28. McLeod R, Khan A R, Noble G A, Latkany P, Jalbrzikowski J, Boyer K. (2006) Severe sulfadiazine hypersensitivity in a child with reactivated congential toxoplasmic chorioretinitis. Pediatr Infect Dis J. 25(3):270-2.
29. McPhillie M, Zhou Y, El Bissati K, Dubey J, Lorenzi H, Capper M, et al (2016) New paradigms for understanding and step changes in treating active and chronic, persistent apicomplexan infections. Sci Rep. 6:29179.
30. Miley G P, Pou S, Winter R, Nilsen A, U Y, Kelly J X, et al (2015) ELQ-300 Prodrugs for Enhanced Delivery and Single-Dose Cure of Malaria AAC 59: 5555-556
31. Muller I, Jex A R, Kappe SHI, Mikolajczak S A, Sattabongkot J, Patrapuvich R, et al Vivax sporozoite consortium. (2019) Transcriptome and histone 1 epigenome of Plasmodium vivax salivary-gland sporozoites point to tight regulatory control and potential mechanisms for liver-stage differentiation. Int J Parasitol 49(7):501-513
32. Murshudov G N, Skubák P, Lebedev A A, Pannu N S, Steiner R A, Nicholls R A, et al., (2011). REFMACS for the refinement of macromolecular crystal structures. Acta Crystallographica Section D: Biological Crystallography, 67(4): 355-367.
33. Ngô H M, Zhou Y, Lorenzi H, Wang K, Kim T K, Zhou Y, et al (2017) Toxoplasma Modulates Signature Pathways of Human Epilepsy, Neurodegeneration & Cancer. Sci Rep. 7(1):11496.
34. Paredes-Santos T C, Martins-Duarte E S, Vitor R W, de Souza W, Attias M, Vommaro R C. (2013) Spontaneous cystogenesis in vitro of a Brazilian strain of Toxoplasma gondii. Parasitol Int. 62(2):181-8.
35. Paredes-Santos T C, Martins-Duarte E S, de Souza W, Attias M, Vommaro R C. (2018) Toxoplasma gondii reorganizes the host cell architecture during spontaneous cyst formation in vitro. Parasitology. 145(8):1027-1038.
36. Phan L, Kasza K, Jalbrzikowski J, Noble A G, Latkany P, Kuo A, et al. (2008) Longitudinal study of new eye lesions in children with toxoplasmosis who were not treated during the first year of life. Am J Ophthalmol. 146(3):375-384.
37. Plouffe D, Brinker A, McNamara C, Henson K, Kato N, Kuhen K, Nagle A, et al (2008) In silico activity profiling reveals the mechanism of action of antimalarials discovered in a high-throughput screen. Proc Natl Acad Sci USA. 105:9059-9064.
38. Pybus B S, Marcsisin S R, Jin X, Deye G, Sousa J C, Li Q, et al (2013) The metabolism of primaquine to its active metabolite is dependent on CYP 2D6. Malaria J. 12:212.
39. St Jean P L, Xue Z, Carter N, Koh G C, Duparc S, Taylor M, et al (2016) Tafenoquine treatment of Plasmodium vivax malaria: suggestive evidence that CYP2D6 reduced metabolism is not associated with relapse in the Phase 2b DETECTIVE trial Malar J 15:97
40. Su G, Morris J H, Demchak B, Bader G D. (2014) Biological network exploration with Cytoscape 3. Current protocols in bioinformatics; 47:8.13.1-8.13.24.
41. Subramanian A, Tamayo P, Mootha V K, Mukherjee S, Ebert B L, Gillette M A, et al. (2005) Gene set enrichment analysis: a knowledge-based approach for interpreting genome-wide expression profiles. Proceedings of the National Academy of Sciences of the United States of America. 102(43):15545-50.
42. Trager, W. & Jensen, J. B., (2005). Human malaria parasites in continuous culture. 1976. The Journal of parasitology, 91(3), pp. 484-486.
43. Vercesi A E, Rodrigues C O, Uyemura S A, Zhong L, Moreno S N J. (1998) Respiration and Oxidative Phosphorylation in the Apicomplexan Parasite Toxoplasma gondii. J. Biol. Chem. 273:31040
44. Vidigal P V T, Santos D V V, Castro F C, Couto J C de F, Vitor R W de A, Brasileiro Filho G. (2002) Prenatal toxoplasmosis diagnosis from amniotic fluid by PCR. Rev Soc Bras Med Trop 35(1):1-6.
45. Walton N M, Sutter B M, Chen H X, Chang L J, Roper S N, Scheffler B, et al. (2006) Derivation and large-scale expansion of multipotent astroglial neural progenitors from adult human brain. Development. 133(18):3671-81.
46. Watts E, Zhao Y, Dhara A, Eller B, Patwardhan A, Sinai A P. (2015) Toxoplasma gondii Bradyzoites within Tissue Cysts Are Dynamic and Replicating Entities In Vivo. Novel approaches reveal that Toxoplasma gondii Bradyzoites within Tissue Cysts Are Dynamic and Replicating Entities In Vivo. MBio. 6(5):e01155-15.
47. Waxman S, Herbert V. (1969) Mechanism of pyrimethamine-induced megaloblastosis in human bone marrow. N Engl J Med. 280(24):1316-9.
48. Zheng S Q, Palovcak E, Armache J P, Verba K A, Cheng Y, Agard D A. (2017) MotionCor2-anisotropic correction of beam-induced motion for improved cryo-electron microscopy. Nat Methods. 14(4): 331-332.
49. Zhang, J, Tan, P, Guo, L, Gong, J, Ma, J, Li, J et al. (2016) p53-dependent autophagic. Cryo-EM of Cellular Machineries. PNAS U.S.A. 2016; 113 (41):11519-11524

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A nanoparticle formulation comprising
an aqueous carrier fluid; and
a dispersion of particles within the aqueous carrier fluid, wherein the particles comprise a hydrophobic material with a surfactant and one or more compounds of formula (I), 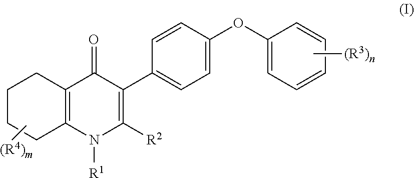
or a pharmaceutically acceptable salt thereof, wherein
m is 0 or 1;
n is 0, 1, or 2;
R¹ is hydrogen or C₁-C₃ alkyl;
R² is hydrogen, C₁-C₃ alkyl, or —CH₂OH;
each R³ is independently halogen, C₁-C₃ alkyl, C₁-C₃ haloalkyl, C1-C₃ alkoxy, C₁-C₃ haloalkoxy; and
each R⁴ is independently C₁-C₃ alkyl, or C₁-C₃ haloalkyl.

2. The nanoparticle formulation of claim 1, wherein
m is 0 or 1;
n is 0, 1, or 2;
R¹ is hydrogen;
R² is hydrogen, methyl, or —CH₂OH:
each R³ is independently halogen, trifluoromethyl, methoxy, or trifluoromethoxy; and
each R⁴ is independently methyl, ethyl, or trifluoromethyl.

3. The nanoparticle formulation of claim 1, compound of formula (I) is

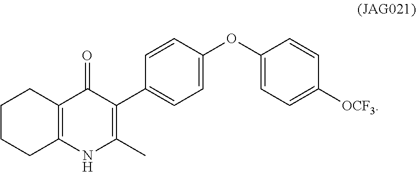 [SMILES: Cc1[nH]c2c(c(=O)c1-c1ccc(Oc3ccc(OC(F)(F)F)cc3)cc1)CCCC2]

4. A nanoparticle formulation comprising
an aqueous carrier fluid, and
a dispersion of particles within the aqueous carrier fluid, wherein the particles comprise a hydrophobic material with a surfactant and one or more compounds selected from: 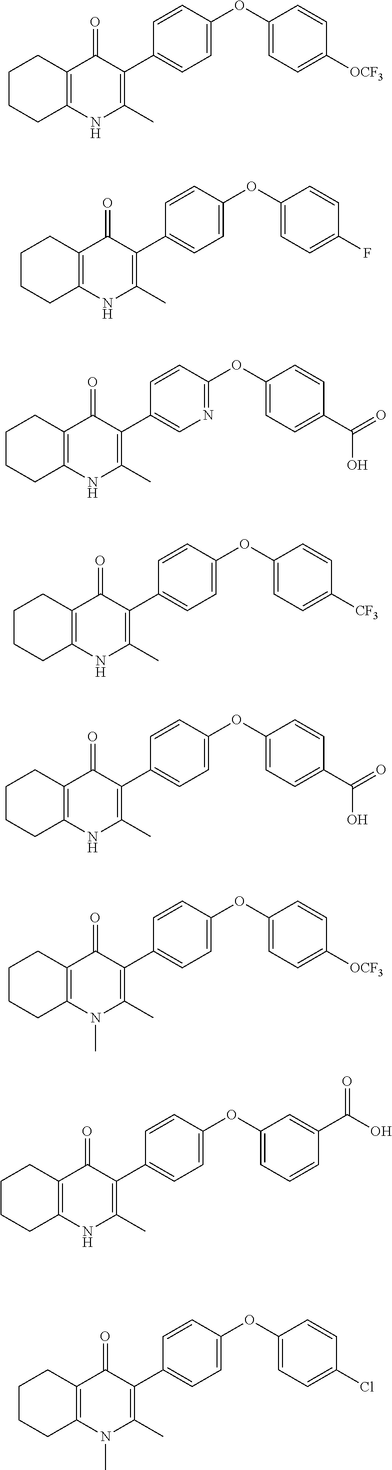 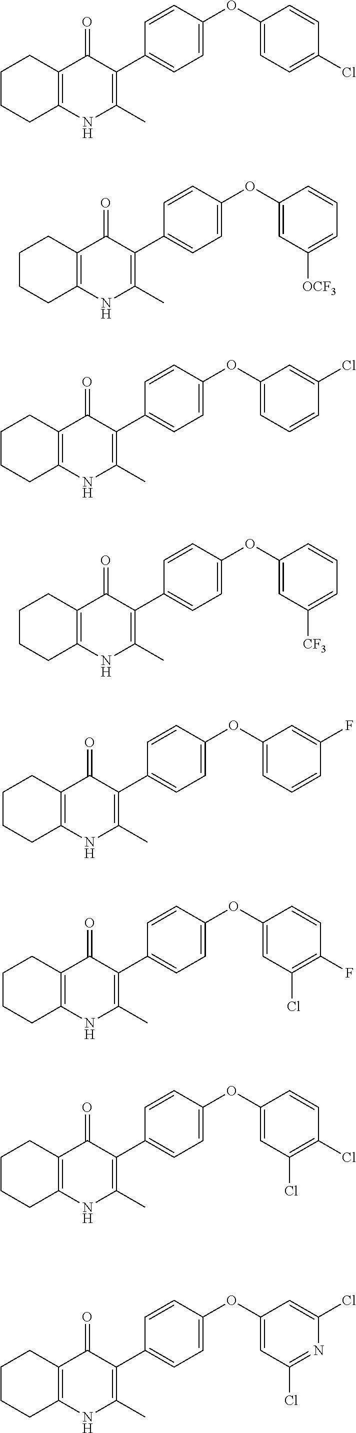 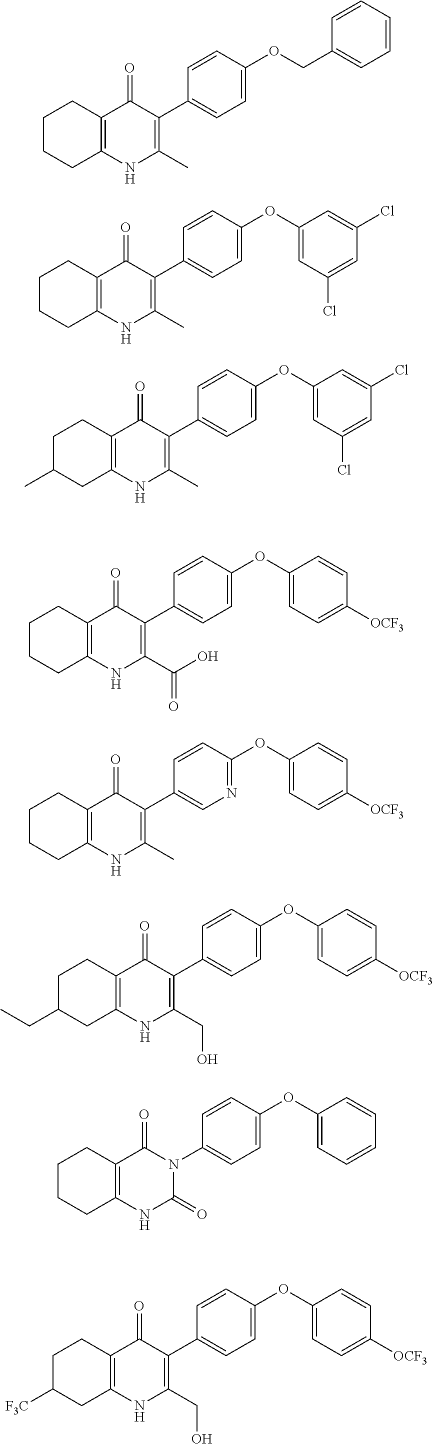 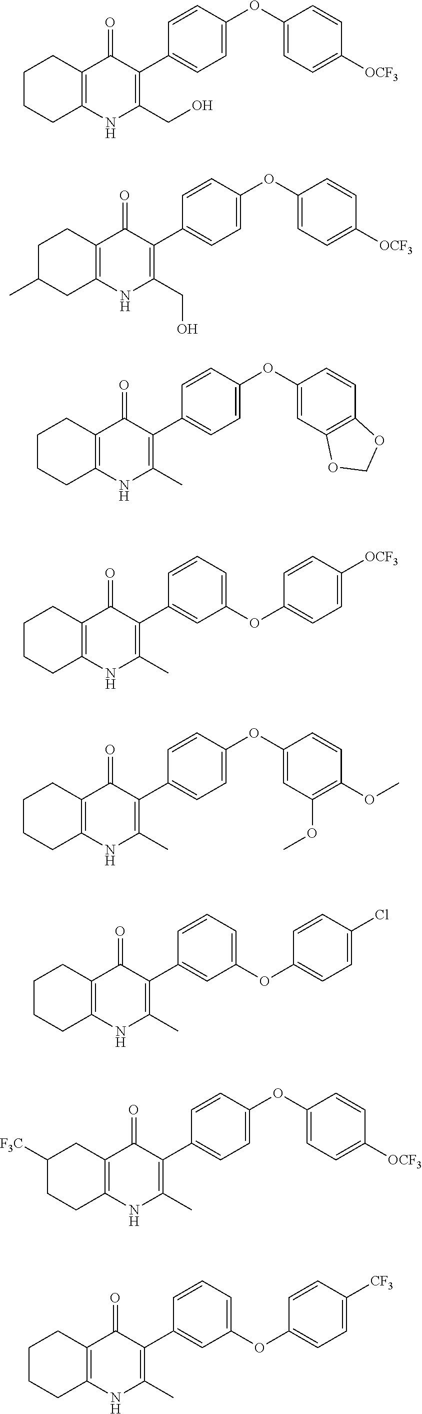 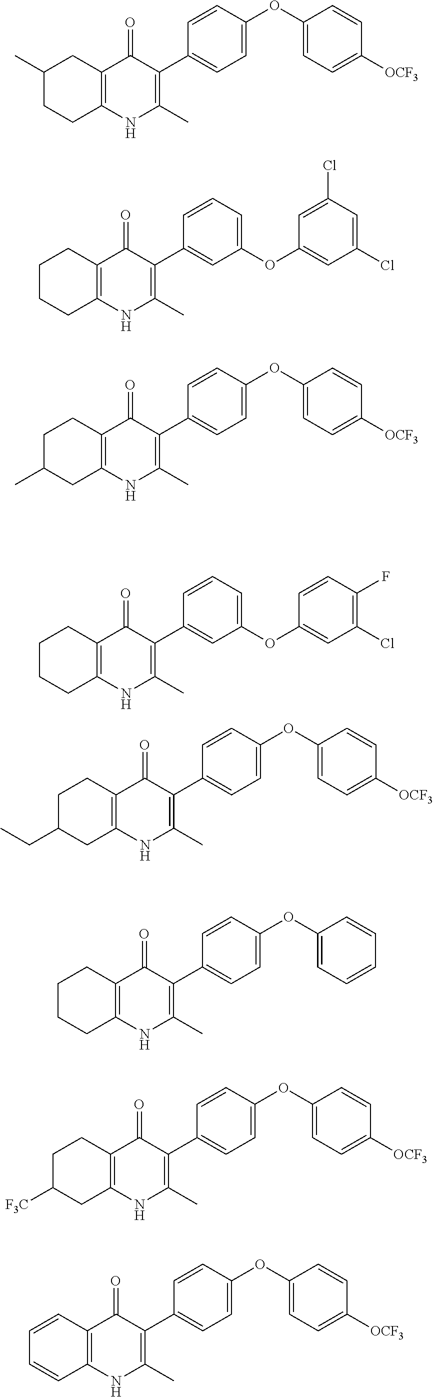 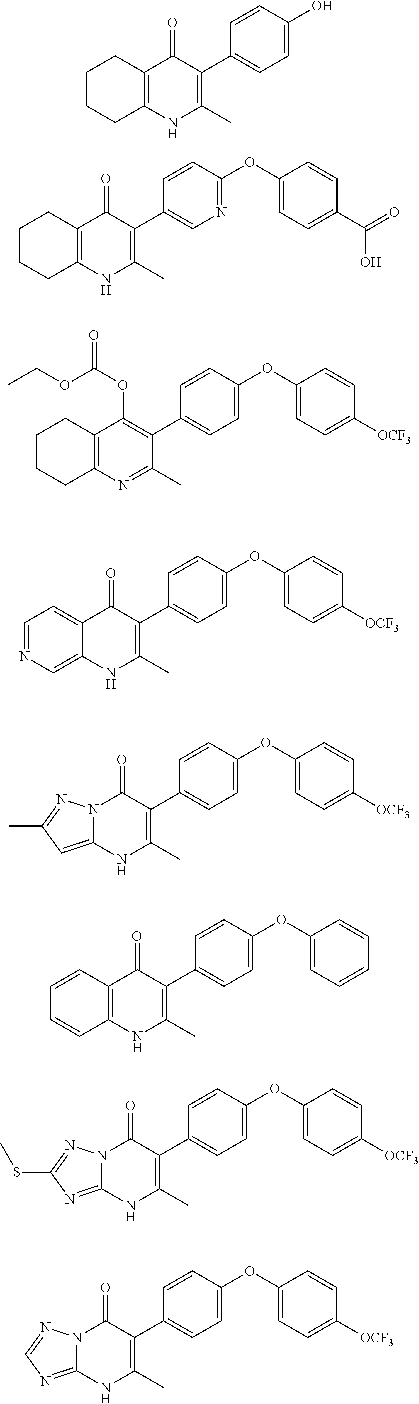 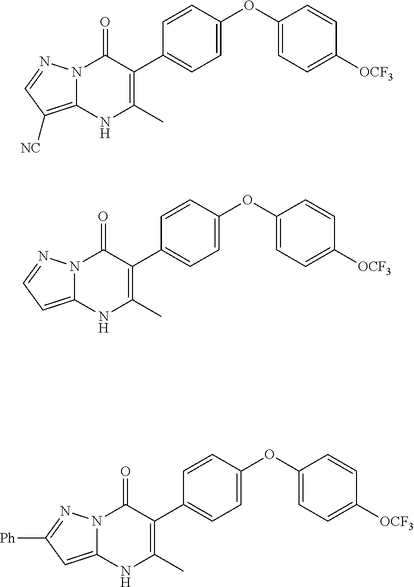 [SMILES: Cc1[nH]c2c(C#N)cnn2c(=O)c1-c1ccc(Oc2ccc(OC(F)(F)F)cc2)cc1 ; Cc1[nH]c2ccnn2c(=O)c1-c1ccc(Oc2ccc(OC(F)(F)F)cc2)cc1 ; Cc1[nH]c2cc(-c3ccccc3)nn2c(=O)c1-c1ccc(Oc2ccc(OC(F)(F)F)cc2)cc1]
or a pharmaceutically acceptable salt thereof.

5. The nanoparticle formulation of any of claims 1-4, wherein the hydrophobic material is hydroxyethyl cellulose (HEC).

6. The nanoparticle formulation of any of claims 1-5, wherein the hydrophobic material is present in an amount in a range of 3 mg/mL to 7 mg/mL (such as about 5 mg/mL).

7. The nanoparticle formulation of any of claims 1-6, wherein the surfactant is polyethylene glycol sorbitan monooleate (such as Tween® 80).

8. The nanoparticle formulation of any of claims 1-7, wherein the surfactant is present in an amount in a range of 1 mg/mL to 3 mg/mL (such as about 2 mg/mL).

9. A compound selected from: 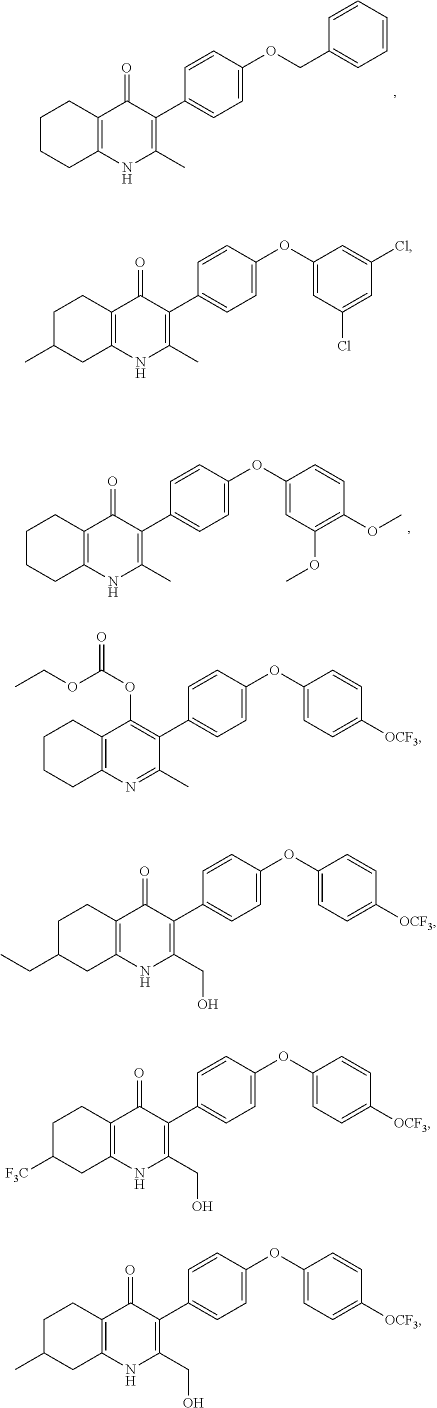 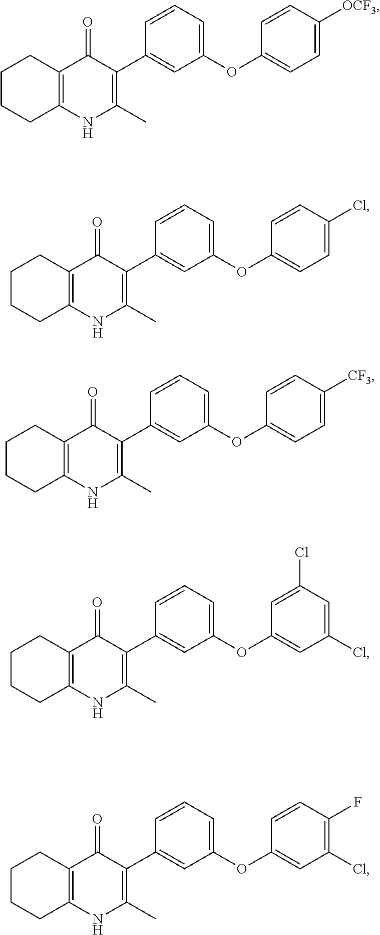 [SMILES: Cc1[nH]c2c(c(=O)c1-c1cccc(Oc3ccc(OC(F)(F)F)cc3)c1)CCCC2 ; Cc1[nH]c2c(c(=O)c1-c1cccc(Oc3ccc(Cl)cc3)c1)CCCC2 ; Cc1[nH]c2c(c(=O)c1-c1cccc(Oc3ccc(C(F)(F)F)cc3)c1)CCCC2 ; Cc1[nH]c2c(c(=O)c1-c1cccc(Oc3cc(Cl)cc(Cl)c3)c1)CCCC2 ; Cc1[nH]c2c(c(=O)c1-c1cccc(Oc3ccc(F)c(Cl)c3)c1)CCCC2]
or a pharmaceutically acceptable salt thereof.

10. A pharmaceutical composition comprising one or more compounds according to claim 9 and a pharmaceutically acceptable carrier, solvent, adjuvant or diluent.

11. A method for treating an apicomplexan parasite infection, comprising administering to a subject in need thereof an amount effective to treat the infection of one or more of modulators of one or more of the genes listed in FIG. 6A or Table 3.

12. The method of claim 12, wherein the one or more modulators comprises one or more inhibitors (of up-regulated genes) or one or more of activators (of down-regulated genes) of one or more of the genes listed in FIG. 6A or Table 3.

13. The method of claim 11 or 12, wherein the method comprises administering to the subject an amount effective to treat the infection of one or more of inhibitors of up-regulated genes listed in FIG. 6A or Table 3.

14. The method of any one of claims 11-13, wherein the method comprises administering to the subject an amount effective to treat the infection of one or more of inhibitors of one or more gene listed in Table 3 as entry No. 213-937.

15. The method of any one of claims 11-14, wherein the method comprises administering to the subject an amount effective to treat the infection of one or more of inhibitors of one or more gene listed in FIG. 6A in Row Nos. 1-10, 12-15, 17, 19-21, 23, 26, 27, 29-31, 33-36, and 41-44.

16. The method of any one of claims 11-15, wherein the method comprises administering to the subject an amount effective to treat the infection of one or more of inhibitors of one or more of the genes listed in FIG. 6A in Row No. 1-10, 12-15, 17, and 19-21.

17. The method of any one of claims 11-16, wherein the method comprises administering to the subject an amount effective to treat the infection of one or more of inhibitors of one or more gene selected from the group consisting of eukaryotic initiation factor-2a kinase (eif2k) gene (IF2K-B), GCN-1 (Row 14, ID #TGME49_231480), MIF4 domain (Row 13, ID #TGME49_269180), hypothetical protein (Row 10, ID #TGME49_206550), and hypothetical protein (Row 3, ID #TGME49_268240).

18. The method of claim 17, wherein the inhibitor comprises an inhibitor selected from the group consisting of an antibody selective for the expressed protein from the one or more genes, and an inhibitory nucleic acid selective for the one or more gene selected from the group consisting of aptamer, small interfering RNA, small internally segmented interfering RNA, short hairpin RNA, microRNA, and antisense oligonucleotides.

19. The method of any one of claims 11-18, wherein the one or more of inhibitors or one or more of activators comprises a compound of formula (I) as described in any of claims 1-3.

20. The method of any one of claims 11-19, wherein the one or more of inhibitors comprises any one of compounds listed in claim 4.

21. The method of any one of claims 11-20, wherein the one or more of inhibitors or one or more of activators comprises any one of compounds as disclosed in International Patent Publication WO 2017/112678 (such as the compounds disclosed in the table at pages 141-145, and claims 20 and 21).

22. The method of any one of claims 11-21, wherein the one or more of inhibitors or one or more of activators comprises an activator of Ribosomal protein RPS13 (Row 24, ID #TGME49_270380).

23. A method for treating an apicomplexan parasite infection, comprising administering to a subject in need thereof an amount effective to treat the infection (i) one or more of eukaryotic initiation factor-2a kinase (eif2k) inhibitors selected from the group consisting of anti-eif2k antibody, anti-eif2k aptamer, eif2k small interfering RNA, eif2k small internally segmented interfering RNA, eif2k short hairpin RNA, eif2k microRNA, and eif2k antisense oligonucleotides, and (ii) one or more compounds of formula (I) as described in any of claims 1-3, or compounds listed in claim 4, or compounds as disclosed in International Patent Publication WO 2017/112678 (such as the compounds disclosed in the table at pages 141-145, and claims 20 and 21).

24. The method of claim 23, wherein (ii) the compound is

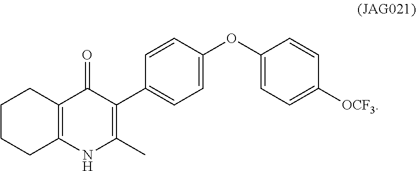 [SMILES: Cc1[nH]c2c(c(=O)c1-c1ccc(Oc3ccc(OC(F)(F)F)cc3)cc1)CCCC2]

25. The method of claim 24, wherein JAG021 is administered as a nanoparticle formulation according to any of claims 5-10.

26. A method for diagnosing an apicomplexan parasite infection, comprising (a) determining an expression level of one or more of the up-regulated and/or down-regulated genes listed in FIG. 6A or Table 3 in a biological sample from a subject; and (b) identifying a subject as having an apicomplexan parasite infection if subject has (i) an expression level of 1, 2, 3, 4, 5, or more up-regulated genes increased relative to a threshold, (ii) an activity level of protein expressed from 1, 2, 3, 4, 5, or more up-regulated genes increased relative to a threshold, (iii) an expression level of 1, 2, 3, 4, 5, or more down-regulated genes decreased relative to a threshold, and/or (iv) an activity level of protein expressed from 1, 2, 3, 4, 5, or more down-regulated genes decreased relative to a threshold.

27. The method of claim 26, comprising determining the expression level of one or more gene listed in Table 3 as entry No. 213-937, and/or determining an activity level of the protein product of one or more gene listed in Table 3 as entry No. 213-937.

28. The method of claim 26 or 27, comprising determining the expression level of one or more gene listed in FIG. 6A in Row Nos. 1-10, 12-15, 17, 19-21, 23, 26, 27, 29-31, 33-36, and 41-44, and/or determining an activity level of the protein product of one or more gene listed in FIG. 6A in Row Nos. 1-10, 12-15, 17, 19-21, 23, 26, 27, 29-31, 33-36, and 41-44.

29. The method of any one of claims 26-28, comprising determining the expression level of one or more gene listed in FIG. 6A in Row No. 1-10, 12-15, 17, and 19-21, and/or determining an activity level of the protein product of one or more gene listed in FIG. 6A in Row No. 1-10, 12-15, 17, and 19-21.

30. The method of any one of claims 26-29, comprising determining the expression level of Ribosomal protein RPS13 (Row 24, ID #TGME49_270380), and/or determining an activity level of the protein product of Ribosomal protein RPS13 (Row 24, ID #TGME49_270380).

31. The method of any one of claims 11-30, wherein the apicomplexan parasite infection is a T. gondii infection.

32. A method for identifying test compounds for apicomplexan parasite therapy, comprising identifying one or more of test compounds that modulate activity of one or more of the genes listed in FIG. 6A or Table 3.

33. The method of claim 32, wherein the method comprises identifying one or more test compounds that reduce activity and/or expression or increase expression of one or more of the genes listed in FIG. 6A or Table 3.

34. The method of claim 32 or 33, wherein the method comprises identifying one or more of test compounds that reduce activity and/or expression.

35. The method of any one of claims 32-34, wherein the method comprises identifying one or more test compounds that reduce activity and/or expression of one or more gene comprises the gene listed in Table 3 as entry No. 213-937.

36. The method of any one of claims 32-35, wherein the method comprises identifying one or more test compounds that reduce activity and/or expression of one or more gene listed in FIG. 6A in Row Nos. 1-10, 12-15, 17, 19-21, 23, 26, 27, 29-31, 33-36, and 41-44.

37. The method of any one of claims 32-36, wherein the method comprises identifying one or more test compounds that reduce activity and/or expression of one or more gene listed in FIG. 6A as Row No. 1-10, 12-15, 17, and 19-21.

38. The method of any one of claims 32-37, wherein the method comprises identifying one or more test compounds that reduce activity and/or expression of one or more genes selected from the group consisting of eukaryotic initiation factor-2a kinase (eif2k) gene (IF2K-B), GCN-1 (Row 14, ID #TGME49_231480), MIF4 domain (Row 13, ID #TGME49_269180), hypothetical protein (Row 10, ID #TGME49_206550), and hypothetical protein (Row 3, ID #TGME49_268240).

39. The method of any one of claims 32-38, wherein the method comprises identifying one or more test compounds that increase activity and/or expression of Ribosomal protein RPS13 (Row 24, ID #TGME49_270380).
